SEQUENCE LISTING (65 sequences)

SEQ ID NO: 1 - protein, 1296 aa, Clostridium botulinum
  source 1..1296: note = Clostridium botulism
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 2 - protein, 1291 aa, Clostridium botulinum
  source 1..1291: note = Clostridium botulism
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 3 - protein, 1291 aa, Clostridium botulinum
  source 1..1291: note = Clostridium botulism
MPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 4 - protein, 1276 aa, Clostridium botulinum
  source 1..1276: note = Clostridium botulism
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 5 - protein, 1251 aa, Clostridium botulinum
  source 1..1251: note = Clostridium botulism
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 6 - protein, 1278 aa, Clostridium botulinum
  source 1..1278: note = Clostridium botulism
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 7 - protein, 1297 aa, Clostridium botulinum
  source 1..1297: note = Clostridium botulism
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 8 - protein, 1288 aa, Clostridium botulinum
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYGELKYNCILNIKYEMDRDKLVDSSGYRSRINIGTGVKFSEIDKNQVQLSNLESSKIEVILNNGVIYNSMYENFSTSFWIRIPKYFRNINNEYKIISCMQNNSGWEVSLNFSNMNSKIIWTLQDTEGIKKTVVFQYTQNINISDYINRWIFVTITNNRLSNSKIYINGRLINEESISDLGNIHASNNIMFKLDGCRDPHRYIWIKYFNLFDKELNKKEIKDLYDNQSNSGILKDFWGDYLQYDKPYYMLNLYDPNKYLDVNNVGIRGYMYLKGPRGRIVTTNIYLNSTLYMGTKFIIKKYASGNKDNIVRNNDRVYINVVVKNKEYRLATNASQAGVEKILSAVEIPDVGNLSQVVVMKSENDQGIRNKCKMNLQDNNGNDIGFIGFHQFNNIAKLVASNWYNRQIGKASRTFGCSWEFIPVDDGWGESSL

SEQ ID NO: 9 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 10 - protein, 1279 aa, Enterococcus faecium
MVTINDLHYSDPIDEDNIINMRIPLYDLEVDDQFINHNVPDLKAFQVFPNVWVVPERYTFYSTMKNLDAPANPSRSSYYDPTYLQSDAEKEVFLQQMILLFKRINSTQEGQQFLNLLSRSIPVPYESNGDVAMGTTQVIKQMDDKGNVLKHRRAHIIIYGPGPDLMAKGSKALTKSRETGRGCMAEIYFSPMYHKTYSTKLTNKNSLVDKSVQEFVPDPAVTLIHELCHGLHALYGIDLGNVGSWEFNSNPNSLFSSWFSSKEAVNFEEVMTFGGEDVKVIKSEIDKKIPGILNLIKTTVEPIINKITDPHDEMLQCLQSKYPSLKGTLGQFFFDDTQLEKDIRDLWMVMNETMFAENLKALTRARYLVPKVENIVQVDILSPNVYTIDKGFNHLSKGFKGQSVSQSYFRKISALARGAVVRACPNPHFSSQRGLSSCIEILEDDLFIMSSKDSFTDTDFSEPSVGPVSYKAKKGADTILDSTLSNYDFSKEINFTSTVPIITVEDPLETDEDVPVISEDRTVYVDDYTTFHFLEAQKIGKEVVPTQTKVVFTTNMEEALFDSKKVYTVFENTASRINEAGTGIANGMMFYQWLKGIVQDFTEEATQKDTFDKISDVTMIVPYLGNILNIGNDIRKGDFMGAVELGGVTILLEAIPELTLPVLIGLTIIEDELEKEQVSQTVYNVLDKRDEKWEEVYGFVKQQWWWMVHTQFETRILHAYQALNHQVEAIKANMTYQLANYRGNQEDKELLEKAIDDTLQSLYYAVDQAMHNIKRFLIQSSKSYLLNQMLPKTKEQLLAFDQQTLRNVNDFINKNQGVLGESLAKDLKKKVEKRLTSLPVFNLEDLPISEFEDLIHSHEIDIQDSEVLNIGVNNGKIQDLSGENTPLTLGENLHIVNGRDNQAVRLNNQLDSKLEIQSRPNIHFTAFEDFSISIWIRCSMLRNNRNRGQKYTIIQQFNKYGWQLAIQDSVFVWTLHDTFNNQIQLTSGSALTNKNYLLQNFWLHITVTNKRSEKSRLYINGVLQDQKDISVLGNCHPKEPILFSIQDNSDPNYFVRFEQFNVYRKALTDSEVNRLYWKYFEGSYLRDVWGERLTYNRDYYMQLSTLPGRGIKREYRTWSGFDYIILSELGTQKIPTHEVTYPKLYQGQKITIHSDGKNLEPHVKSNKNIRLKIDDFYIGVVNPFKLPEWRPESGAYVVTTYNHAEDLCLYFRTRSSSQSLYYGQLIMNDGRNKSLLNYTLKGSTYWIWSSAWYYENYNTSSKTAGNWYFIPVDEGWKED

SEQ ID NO: 11 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 12 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPWRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 13 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPFRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 14 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPHRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 15 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPLRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 16 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGERSL

SEQ ID NO: 17 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESPL

SEQ ID NO: 18 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGERPL

SEQ ID NO: 19 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPWRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGERPL

SEQ ID NO: 20 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPFRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGERPL

SEQ ID NO: 21 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPHRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGERPL

SEQ ID NO: 22 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPLRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGERPL

SEQ ID NO: 23 - protein, 410 aa, Clostridium botulinum
DLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 24 - protein, 410 aa, synthetic construct
DLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPWRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 25 - protein, 410 aa, synthetic construct
DLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPFRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 26 - protein, 410 aa, synthetic construct
DLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPHRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 27 - protein, 410 aa, synthetic construct
DLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPLRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 28 - protein, 410 aa, synthetic construct
DLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGERSL

SEQ ID NO: 29 - protein, 410 aa, synthetic construct
DLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESPL

SEQ ID NO: 30 - protein, 410 aa, synthetic construct
DLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGERPL

SEQ ID NO: 31 - protein, 410 aa, synthetic construct
DLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPWRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGERPL

SEQ ID NO: 32 - protein, 410 aa, synthetic construct
DLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPFRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGERPL

SEQ ID NO: 33 - protein, 410 aa, synthetic construct
DLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPHRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGERPL

SEQ ID NO: 34 - protein, 410 aa, synthetic construct
DLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPLRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGERPL

SEQ ID NO: 35 - protein, 857 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYN

SEQ ID NO: 36 - protein, 870 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNIND

SEQ ID NO: 37 - protein, 862 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDNFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFKQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFN

SEQ ID NO: 38 - protein, 844 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFF

SEQ ID NO: 39 - protein, 863 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLY

SEQ ID NO: 40 - protein, 858 aa, Clostridium botulinum
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNY

SEQ ID NO: 41 - protein, 889 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYK

SEQ ID NO: 42 - protein, 874 aa, Enterococcus faecium
MVTINDLHYSDPIDEDNIINMRIPLYDLEVDDQFINHNVPDLKAFQVFPNVWVVPERYTFYSTMKNLDAPANPSRSSYYDPTYLQSDAEKEVFLQQMILLFKRINSTQEGQQFLNLLSRSIPVPYESNGDVAMGTTQVIKQMDDKGNVLKHRRAHIIIYGPGPDLMAKGSKALTKSRETGRGCMAEIYFSPMYHKTYSTKLTNKNSLVDKSVQEFVPDPAVTLIHELCHGLHALYGIDLGNVGSWEFNSNPNSLFSSWFSSKEAVNFEEVMTFGGEDVKVIKSEIDKKIPGILNLIKTTVEPIINKITDPHDEMLQCLQSKYPSLKGTLGQFFFDDTQLEKDIRDLWMVMNETMFAENLKALTRARYLVPKVENIVQVDILSPNVYTIDKGFNHLSKGFKGQSVSQSYFRKISALARGAVVRACPNPHFSSQRGLSSCIEILEDDLFIMSSKDSFTDTDFSEPSVGPVSYKAKKGADTILDSTLSNYDFSKEINFTSTVPIITVEDPLETDEDVPVISEDRTVYVDDYTTFHFLEAQKIGKEVVPTQTKVVFTTNMEEALFDSKKVYTVFENTASRINEAGTGIANGMMFYQWLKGIVQDFTEEATQKDTFDKISDVTMIVPYLGNILNIGNDIRKGDFMGAVELGGVTILLEAIPELTLPVLIGLTIIEDELEKEQVSQTVYNVLDKRDEKWEEVYGFVKQQWWWMVHTQFETRILHAYQALNHQVEAIKANMTYQLANYRGNQEDKELLEKAIDDTLQSLYYAVDQAMHNIKRFLIQSSKSYLLNQMLPKTKEQLLAFDQQTLRNVNDFINKNQGVLGESLAKDLKKKVEKRLTSLPVFNLEDLPISEFEDLIHSHEIDIQDSEVLNIGVNN

SEQ ID NO: 43 - protein, 862 aa, Clostridium botulinum
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYI

SEQ ID NO: 44 - protein, 1281 aa, synthetic construct
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 45 - protein, 1294 aa, synthetic construct
MPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 46 - protein, 1286 aa, synthetic construct
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDNFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFKQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 47 - protein, 1268 aa, synthetic construct
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 48 - protein, 1287 aa, synthetic construct
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 49 - protein, 1286 aa, synthetic construct
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYIIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 50 - protein, 1282 aa, synthetic construct
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 51 - protein, 1313 aa, synthetic construct
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 52 - protein, 1298 aa, synthetic construct
MVTINDLHYSDPIDEDNIINMRIPLYDLEVDDQFINHNVPDLKAFQVFPNVWVVPERYTFYSTMKNLDAPANPSRSSYYDPTYLQSDAEKEVFLQQMILLFKRINSTQEGQQFLNLLSRSIPVPYESNGDVAMGTTQVIKQMDDKGNVLKHRRAHIIIYGPGPDLMAKGSKALTKSRETGRGCMAEIYFSPMYHKTYSTKLTNKNSLVDKSVQEFVPDPAVTLIHELCHGLHALYGIDLGNVGSWEFNSNPNSLFSSWFSSKEAVNFEEVMTFGGEDVKVIKSEIDKKIPGILNLIKTTVEPIINKITDPHDEMLQCLQSKYPSLKGTLGQFFFDDTQLEKDIRDLWMVMNETMFAENLKALTRARYLVPKVENIVQVDILSPNVYTIDKGFNHLSKGFKGQSVSQSYFRKISALARGAVVRACPNPHFSSQRGLSSCIEILEDDLFIMSSKDSFTDTDFSEPSVGPVSYKAKKGADTILDSTLSNYDFSKEINFTSTVPIITVEDPLETDEDVPVISEDRTVYVDDYTTFHFLEAQKIGKEVVPTQTKVVFTTNMEEALFDSKKVYTVFENTASRINEAGTGIANGMMFYQWLKGIVQDFTEEATQKDTFDKISDVTMIVPYLGNILNIGNDIRKGDFMGAVELGGVTILLEAIPELTLPVLIGLTIIEDELEKEQVSQTVYNVLDKRDEKWEEVYGFVKQQWWWMVHTQFETRILHAYQALNHQVEAIKANMTYQLANYRGNQEDKELLEKAIDDTLQSLYYAVDQAMHNIKRFLIQSSKSYLLNQMLPKTKEQLLAFDQQTLRNVNDFINKNQGVLGESLAKDLKKKVEKRLTSLPVFNLEDLPISEFEDLIHSHEIDIQDSEVLNIGVNNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 53 - protein, 6 aa, synthetic construct
LVPRGS

SEQ ID NO: 54 - protein, 7 aa, synthetic construct
ENLYFQG

SEQ ID NO: 55 - protein, 8 aa, synthetic construct
LEVLFQGP

SEQ ID NO: 56 - protein, 4 aa, synthetic construct
IEGR

SEQ ID NO: 57 - protein, 4 aa, synthetic construct
IDGR

SEQ ID NO: 58 - protein, 5 aa, synthetic construct
DDDDK

SEQ ID NO: 59 - protein, 8 aa, synthetic construct
AHREQIGG

SEQ ID NO: 60 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPYRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 61 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPARYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 62 - protein, 410 aa, synthetic construct
DLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPYRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 63 - protein, 410 aa, synthetic construct
DLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPARYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 64 - protein, 1309 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSASLVPRGSGGAAALDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 65 - protein, 1309 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSASLVPRGSGGAAALDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPHRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGERPL

(19) United States
(12) Patent Application Publication
(10) Pub. No.: US 2026/0103697 A1
(43) Pub. Date: Apr. 16, 2026

(54) MODIFIED BONT/A2 RECEPTOR-BINDING DOMAINS

(71) Applicant: The Children's Medical Center Corporation, Boston, MA (US)

(72) Inventors: Min Dong, Weatogue, CT (US); Pyung-Gang Lee, Boston, MA (US)

(73) Assignee: The Children's Medical Center Corporation, Boston, MA (US)

(21) Appl. No.: 19/116,066

(22) PCT Filed: Sep. 29, 2023
(86) PCT No.: PCT/US2023/075514

§ 371 Date: Mar. 27, 2025

Related U.S. Application Data
(60) Provisional application No. 63/411,538, filed on Sep. 29, 2022.

(51) Int. Cl. C12N 9/52; A61K 8/66; A61K 38/00; A61P 21/00; A61Q 19/08; C07K 14/33

(52) U.S. Cl. CPC C12N 9/52; A61K 8/66; A61P 21/00; A61Q 19/08; C07K 14/33; A61K 38/00; C07K 2319/70; C12Y 304/24069

(57) ABSTRACT

This application relates to, in part, modified botulinum Neurotoxins (BoNTs) comprising modified BoNT/A2 receptor binding domains with increased receptor binding activity, and methods of treatment using the modified BoNT.

### RELATED APPLICATIONS

This application claims the benefit under 35 U.S.C. § 119(e) of U.S. provisional application No. 63/411,538, filed Sep. 29, 2022, the entire contents of which are incorporated herein by reference.

### REFERENCE TO AN ELECTRONIC SEQUENCE LISTING

The contents of the electronic sequence listing (C123370261W000-SEQ-ARM.xml; Size: 117,045 bytes; and Date of Creation: Sep. 29, 2023) is herein incorporated by reference in its entirety.

### BACKGROUND

In recent years, Clostridial botulinum neurotoxin (BoNT) have been widely used to treat a growing list of medical conditions: local injections of minute amount of toxins can attenuate neuronal activity in targeted regions, which can be beneficial in many medical conditions as well as for cosmetic purposes. To date, BoNT serotype A (BoNT/A) and BoNT serotype B (BoNT/B) are the only two BoNTs that are currently FDA-approved for use in humans. As the application of BoNTs grows, limitations and adverse effects have been reported. The major limitation is the generation of neutralizing antibodies in patients, which renders future treatment ineffective. Termination of BoNT usage often leaves patients with no other effective ways to treat/relieve their disorders. Adverse effects associated with BoNT use range from transient nonserious events such as ptosis and diplopia to life-threatening events even death. The limitations and adverse effects of BoNTs are largely correlated with dose. Modified BoNTs with improved specificity for neurons that maintain the same level of toxin activity with lower dose is highly desired.

### SUMMARY

The limitations and adverse effects of BoNTs are largely correlated with dose. Provided herein are modified BoNTs comprising BoNT/A2 receptor binding domains with improved binding affinity. Thus, in some embodiments, the modified BoNTs described herein may be administered at lower dose than wildtype BoNT proteins, which is expected to reduce the possibility of the generation of neutralizing antibodies in patients and adverse side effects.

Some aspects of the present disclosure provide modified Clostridial botulinum neurotoxin (BoNT) polypeptides comprising a modified receptor binding domain of Clostridial botulinum serotype A1 (BoNT/A2). In some aspects, the present disclosure provides modified receptor binding domains of BoNT/A2. In some aspects, the present disclosure provides chimeric BoNT polypeptides comprising a modified receptor binding domain of BoNT/A2. In some embodiments, a BoNT comprising the modified receptor binding domain of BoNT/A2 increases binding of the BoNT to a BoNT receptor protein compared to a BoNT comprising a wildtype BoNT/A2 receptor binding domain.

In some aspects, this application discloses a modified Clostridial botulinum neurotoxin (BoNT) polypeptide comprising a modified receptor binding domain of Clostridial botulinum serotype A2 (BoNT/A2) comprising one or more amino acid substitutions at positions corresponding to 1064, 1294, and 1295 of SEQ ID NO: 1.

In some embodiments, the modified receptor binding domain comprises an amino acid substitution at a position corresponding to 1064 in SEQ ID NO: 1. In some embodiments, the amino acid substitution corresponds to R1064A, R1064L, R1064Y, R1064W, R1064F, or R1064H. In some embodiments, the amino acid substitution corresponds to R1064W, R1064F, or R1064H.

In some embodiments, the modified receptor binding domain comprises an amino acid substitution at a position corresponding to 1294 in SEQ ID NO: 1. In some embodiments, the amino acid substitution corresponds to S1294R.

In some embodiments, the modified receptor binding domain comprises an amino acid substitution at a position corresponding to 1295 in SEQ ID NO: 1. In some embodiments, the amino acid substitution corresponds to S1295P.

In some embodiments, the modified receptor binding domain comprises amino acid substitutions at positions corresponding to 1294 and 1295 in SEQ ID NO: 1. In some embodiments, the amino acid substitutions correspond to S1294R and S1295P in SEQ ID NO: 1

In some embodiments, the modified receptor binding domain comprises amino acid substitutions at positions corresponding to 1064, 1294 and 1295 in SEQ ID NO: 1. In some embodiments, the amino acid substitutions correspond to R1064A, R1064L, R1064H, R1064W, R1064F, or R1064Y; and S1294R and S1295P in SEQ ID NO: 1. In some embodiments, the amino acid substitutions correspond to R1064H, S1294R and S1295P in SEQ ID NO: 1. In some embodiments, the amino acid substitutions correspond to R1064W, S1294R and S1295P in SEQ ID NO: 1. In some embodiments, the amino acid substitutions correspond to R1064F, S1294R and S1295P in SEQ ID NO: 1.

In some embodiments, the modified receptor binding domain comprises an amino acid sequence that is at least 80% identical to the amino acid sequence of any one of SEQ ID NO: 12-22, SEQ ID NOs: 24-34, or SEQ ID NOs: 60-63. In some embodiments, the modified receptor binding domain comprises the amino acid sequence of any one of SEQ ID NO: 12-22, SEQ ID NOs: 24-34, or SEQ ID NOs: 60-63.

In some embodiments, the modified BoNT polypeptide further comprises a protease domain and a translocation domain from BoNT/A2. In some embodiments, the modified BoNT polypeptide further comprises a protease domain and a translocation domain from a second BoNT. In some embodiments, the second BoNT is of serotype B, C, D, E, F, G, H, X, or En.

In some embodiments, the amino acid substitutions do not correspond to P1063T or P1063T and R1064H.

In some embodiments, the modified BoNT polypeptide comprises an amino acid sequence that is at least 80% identical to the amino acid sequence of SEQ ID NO: 21 or 33. In some embodiments, the modified BoNT polypeptide comprises an amino acid sequence that is at least 95% identical to the amino acid sequence of SEQ ID NO: 21 or 33. In some embodiments, the modified BoNT polypeptide comprises the amino acid sequence of SEQ ID NO: 21 or 33.

In some embodiments, the modified BoNT polypeptide comprises an amino acid sequence that is at least 80% identical to SEQ ID NO: 65. In some embodiments, the modified BoNT polypeptide comprises an amino acid sequence that is at least 95% identical to SEQ ID NO: 65. In some embodiments, the modified BoNT polypeptide comprises the amino acid sequence of SEQ ID NO: 65.

In some aspects, this application discloses a nucleic acid molecule comprising a polynucleotide encoding a modified BoNT polypeptide as described herein.

In some aspects, this application discloses a nucleic acid vector comprising a nucleic acid molecule described herein.

In some aspects, this application discloses a cell comprising a nucleic acid molecule described herein or a nucleic acid vector described herein. In some aspects, this application discloses a cell expressing a modified BoNT polypeptide described herein.

In some aspects, this application discloses a method of producing a modified BoNT polypeptide, the method comprising the steps of culturing the cells disclosed herein under conditions wherein the modified BoNT polypeptide is produced.

In some embodiments, the method further comprises recovering the modified BoNT polypeptide from the culture.

In some aspects, this application discloses a pharmaceutical composition comprising the modified BoNT polypeptide as described herein. In some embodiments, the pharmaceutical composition further comprises a pharmaceutically acceptable excipient.

In some aspects, this application discloses a kit comprising a pharmaceutical composition as described herein and directions for therapeutic administration of the pharmaceutical composition.

In some aspects, this application discloses a method of treating a condition, the method comprising administering a therapeutically effective amount of the modified BoNT polypeptide described herein, or a pharmaceutical composition described herein to a subject to treat the condition. In some embodiments, the condition is associated with overactive neurons or glands. In some embodiments, the condition is selected from the group consisting of: depression, spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions, secretory disorders, pain from muscle spasms, headache pain, dermatological or aesthetic/cosmetic conditions, obesity/reduced appetite. In some embodiments, the condition is not associated with unwanted neuronal activity. In some embodiments, the condition is selected from the group consisting of: psoriasis, allergy, haemophagocytic lymphohistiocytosis, and alcoholic pancreatic disease. In some embodiments, the administering is via injection to where unwanted neuronal activity is present.

In some embodiments, this application discloses a modified BoNT polypeptide as described herein or a pharmaceutical composition as described herein, for use in treating a condition associated with unwanted neuronal activity.

In some embodiments, this application discloses a modified BoNT polypeptide described herein or the pharmaceutical composition described herein, for use in medicine.

In some embodiments, this application discloses a modified BoNT polypeptide described herein or a pharmaceutical composition described herein, for cosmetic use.

### BRIEF DESCRIPTION OF DRAWINGS

FIGS. 1A and 1B show modified receptor binding domains of BoNT-A2 (Hc/A2 variants) bind to glycosylated synaptic vesicle glycoprotein 2C (SV2C) better than wildtype BoNT/A2 using a Bio-layer interferometry (BLI) assay. SA biosensors were exposed to three or four different concentrations of Hcs (0.3-10 μM) for 120 seconds (association phase), then washed for 200 seconds (dissociation phase). S1294R/S1295P and R1064F enhanced binding saturation, while S1294R/S1295P, R1064H, R1064W, R1064Y and S1295P showed reduced dissociation, which were cooperatively reflected in reduced K_{D} values (better binding), even better than He/A1 (also see Table 2).

FIGS. 2A-2D show modified full length BoNT/A2 are more active on neurons than wildtype BoNT/A2. FIGS. 2A and 2B Sortase ligation of protease domain and transmembrane domain of BoNT/A2 (LCHn/A2) and He/A2 variants to generate full length toxins. The full-length toxins were quantified based on protein density on the gel using BSA as a standard. The full length BoNTs were also activated with thrombin, which was verified on the gel with or without 2-mercaptoethanol (2-ME). FIG. 2C-2D show that modified BoNT/A2 proteins have higher activity on neurons than wildtype BoNT when administered at the same concentration. Two weeks cultured rat cortical neurons were incubated with ligated full-length toxins for 12 h. Cleaved and uncleaved SNAP25 of neuron lysates were detected in an immunoblotting assay. A221 (LCHn from A2 and He from A1) and A222 (BoNT/A2) showed similar cleavage, but A222 with R1064H or S1294R/S1295P showed slightly higher cleavage. Addition of either R1064H or R1064W onto S1294R/S1295P resulted in 3 times higher cleavage efficiency compared to A222 wild-type.

FIGS. 3A-3B shows evaluation of mutant toxins in vivo using DAS assays. FIG. 3A shows the DAS results of BoNT/A2 WT (wildtype) and BoNT/A2 mutant A2-HRP (R1064H/S1294R/S1295P) with 2 and 8 pg injection. A2-HRP showed similar in vivo paralytic activity compared to BoNT/A2 wildtype. The similarity was conserved in both 2 and 8 pg injection doses. FIG. 3B shows each 20 pg of A2 wildtype and A2-HRP were injected to mice and DAS scores were recorded. A2-wildtype injected mice were euthanized due to severe systemic responses, while A2-HRP raised minor systemic responses that all the injected mice could be monitored until full recovery (˜40 days).

FIGS. 4A-4B shows body weight changes in mice injected with 8 and 20 pg of A2-WT and A2-HRP, respectively. FIG. 4A shows 8 pg of A2-WT injection resulted in ˜20% loss of body weight, while 8 pg of A2-HRP injection resulted in ˜5% loss of body weight. FIG. 4B shows 20 pg of A2-HRP injection resulted in ˜15% loss of body weight, but 20 pg of A2-WT raised severe systemic responses that the monitoring was terminated before recording body weight loss. All body weight loss was recorded at the most, usually two or three days after injection.

### DETAILED DESCRIPTION

Clostridium botulinum neurotoxins (BoNTs) are a family of bacterial toxins produced by clostridium bacteria, with seven well-established serotypes (BoNT/A-G) and two recently discovered serotypes X and EN described in U.S. patent application Ser. Nos. 16/315,698 and 16/651,720 (both incorporated herein by reference). They are one of the most dangerous potential bio-terrorism agents, classified as a “Category A” select agent by Center for Disease Control (CDC) of United States. These toxins are produced as a single polypeptide and can be separated by bacterial or host proteases into a light chain (LC, ˜50 kDa) and a heavy chain (HC, ˜100 kDa). The two chains remain connected via an inter-chain disulfide bond. The HC contains two sub-domains: the N-terminal HN domain that mediates translocation of the LC across endosomal membranes, and the C-terminal HC domain that mediates binding to receptors on neurons. The inter-chain disulfide bond is reduced once the LC translocates into the cytosol. Released LC acts as a protease to specifically cleave a set of neuronal proteins: BoNT/A, C, and E cleave at distinct sites on a protein known as SNAP-25; BoNT/B, D, F, and G cleave at different sites on a vesicle protein VAMP; and BoNT/C also cleaves a transmembrane protein syntaxin. These three proteins form a complex, known as SNARE complex, which is essential for release of neurotransmitters. Cleavage of any one of these three SNARE proteins blocks neurotransmitters release from neurons, thus paralyzing muscles. Recently discovered BoNT/X cleaves SNARE proteins like other BoNTs, but also cleaves non-canonical substrates VAMP4, VAMP5 and Ykt6. Recently discovered BoNT/EN cleaves VAMP1/2/3 and several other SNARE proteins including SNAP-25, SNAP-23, syntaxin 1B and syntaxin 4.

BoNTs are the most potent toxins known and cause the human and animal disease known as botulism. The major form of botulism is caused by ingesting food contaminated with BoNTs (food botulism). Other forms also exist such as infant botulism, which is due to colonization of the intestine by toxin-producing bacteria in infants. BoNTs are always produced together with another 150 kDa protein known as NTNHA (non-toxic non-hemagglutinin protein), which forms a pH-dependent complex with BoNTs and protects BoNTs from proteases in the gastrointestinal tract.

Because local injections of minute amounts of toxins can attenuate neuronal activity in targeted regions, BoNTs have been used to treat a growing list of medical conditions, including muscle spasms, chronic pain, overactive bladder problems, as well as for cosmetic applications. The market for BoNTs has already surpassed $3 billion in 2018. Among the seven types of BoNT toxins, BoNT/A and BoNT/B are the two toxins that are currently FDA-approved for use in humans. BoNT/A is the dominant type used for both medical and cosmetic applications, marketed as Botox from Allergan Inc., Dysport from IPSEN Inc., and Xeomin from Merz Inc. BoNT/B is marketed as Myobloc by USWorld Med. BoNT/A has different subtypes including BoNT/A1, BoNT/A2, BoNT/A3, BoNT/A4, BoNT/A5, BoNT/A6, BoNT/A7, and BoNT/A8.

As the application of BoNTs grows, limitations and adverse effects have been reported. The major limitation is the generation of neutralizing antibodies in patients, which renders future treatment ineffective. Termination of BoNT usage often leaves patients with no other effective ways to treat or relieve their disorders. The probability of antibody responses is directly related to both toxin doses and the frequency of injection. Therefore, this limitation mainly occurs in treating muscle spasms, which involves high dose of toxins. Consistently, antibody responses have not been observed in cosmetic applications, which use extremely low toxin doses.

The major adverse effects are also often associated with treating muscle spasms, but not cosmetic applications. This is because the adverse effects are largely due to diffusion of toxins to other regions of the body and the possibility of toxin diffusion is directly related to injected doses. The adverse effects range from transient non-serious events such as ptosis and diplopia to life-threatening events even death. In a petition letter filed in 2008 by Dr. Sidney Wolfe to FDA, a total of 180 serious adverse events, including 16 deaths have been documented. As a result, FDA now requires the “Black box warning” on all BoNT products, highlighting the risk of the spread of toxins, following similar warnings issued by the European Union.

Because both the generation of neutralizing antibodies and toxin diffusion are directly related to injected doses, lowering toxin doses (while maintaining the same levels of toxin activity) is highly desired, which means the efficacy of individual toxin molecules to induce local muscle paralysis has to be enhanced. Such modified BoNTs with improved local efficacy would also reduce any potential off-target effects due to toxin diffusion to other regions.

The action of BoNTs has three major steps: (1) receptor binding: these toxins target motor nerve terminals by first binding specifically to their receptors expressed in neurons; (2) translocation: after binding to receptors, BoNTs enter cells via receptor-mediated endocytosis into endosomes, and the low pH conditions within endosomes then induce conformational changes of toxin, resulting in its penetration of endosomal membrane and release of its protease domain into the cytosol of neurons; (3) substrate cleavage: within the cytosol of neurons, the released protease domain of BoNTs then cleave proteins required for synaptic transmission, therefore blocking neurotransmission 2. Corresponding to these three steps of action, BoNTs are composed of three functional domains2: (1) the C-terminal receptor-binding domain (HC, ˜50 kDa); (2) the membrane translocation domain in the middle (HN, ˜50 kDa); (3) the N-terminal protease domain (also known as light chain, LC, ˜50 kDa). The HC and HN together form the heavy chain (HC, ˜100 kDa).

Receptor-binding appears to be a rate-limiting step. For instance, enhancing the ability of BoNTs to recognize their neuronal receptors will facilitate absorbance of toxins into neurons at the injection site, therefore shielding toxins from triggering immune responses and also preventing their diffusion. Enhanced affinity and specificity to neuronal receptors will also reduce potential off-target effects due to non-specific entry into other cell types. The receptors for most BoNTs have been identified in recent years. BoNT/B, G, and a mosaic toxin DC share two homologous synaptic vesicle proteins synaptotagmin I and II (Syt I/II) as their receptors 9-16. Another family of synaptic vesicle protein SV2 acts as receptors for BoNT/A, E, D, and potentially F. In addition to protein receptors, all BoNTs require lipid co-receptor gangliosides, which are abundant on neuronal surfaces.

### Modified BoNTs

Accordingly, some aspects of the present disclosure provide modified Clostridial botulinum neurotoxins (BoNT) comprising a modified receptor binding domain of Clostridial botulinum serotype A2 (BoNT/A2). In some embodiments, a BoNT comprising the modified receptor binding domain of a BoNT/A2 increases binding of the BoNT to a BoNT receptor protein compared to a BoNT comprising a wildtype BoNT/A2 receptor binding domain. In some embodiments, a BoNT comprising the modified receptor binding domain of BoNT/A2 reduces systemic toxicity at a dosage that induces the same degree of local paralysis as a BoNT comprising a wildtype BoNT/A2 receptor binding domain. In some embodiments, a BoNT comprising the modified receptor binding domain of a BoNT/A2 increases local paralysis compared to a BoNT comprising a wildtype BoNT/A2 receptor binding domain.

As used herein, the term “Clostridial botulinum neurotoxin (BoNT)” encompasses any polypeptide or fragment from a botulinum neurotoxin. In some embodiments, the term BoNT refers to a full-length BoNT. In some embodiments, the term BoNT refers to a fragment of the BoNT that can execute the overall cellular mechanism whereby a BoNT enters a neuron and inhibits neurotransmitter release. In some embodiments, the term BoNT simply refers to a fragment of the BoNT, without requiring the fragment to have any specific function or activity. Other terms that may be used throughout the present disclosure for “Clostridial botulinum neurotoxins” may be BoNTs, botulinum toxins, or C. botulinum toxins. It is to be understood that these terms are used interchangeably.

A “modified Clostridial botulinum neurotoxin (BoNT)” encompasses a BoNT comprising any modifications in the amino acid sequence, e.g., truncation, addition, amino acid substitution, and any combination thereof. For example, a BoNT/A2 comprising amino acid substitution mutation in amino acid position 1064 or 1294 is a modified BoNT. In another example, a fragment or a domain of the full-length BoNT (e.g., the receptor binding domain) is considered a modified BoNT. In some embodiments, a domain of the BoNT may also comprise amino acid substitution mutation(s), e.g., a receptor binding domain comprising substitution mutation at positions corresponding to 1064 or 1294 of the full-length BoNT. In some embodiments, a modified BoNT may include one or more substitutions to any amino acid other than the wild type residue normally found at positions R1064, S1294, and S1295 of SEQ ID NO: 1. Such substitutions may be replacement with non-polar (hydrophobic) amino acids, such as alanine, valine, leucine, isoleucine, methionine, phenylalanine, tryptophan, and proline. Substitutions may be replacement with polar (hydrophilic) amino acids such as glycine, serine, threonine, cysteine, tyrosine, asparagine, and glutamine. Substitutions may be replacement with electrically charged amino acids, e.g., negatively electrically charged amino acids such as aspartic acid and glutamic acid and positively electrically charged amino acids such as lysine, arginine, and histidine.

As used herein, the term “Clostridial botulinum neurotoxin (BoNT) protease domain” means a BoNT domain that can execute the enzymatic target modification step of the intoxication process. Thus, a BoNT protease domain specifically targets a C. botulinum toxin substrate and encompasses the proteolytic cleavage of a C. botulinum toxin substrate, such as, e.g., SNARE proteins like a SNAP-25 substrate, a VAMP substrate and a Syntaxin substrate.

As used herein, the term “Clostridial botulinum neurotoxin (BoNT) translocation domain” or “HN” means a BoNT domain that can execute the translocation step of the intoxication process that mediates BoNT light chain translocation. Thus, an HN facilitates the movement of a BoNT light chain across a membrane into the cytoplasm of a cell. Non-limiting examples of a Hn include a BoNT/A HN, a BoNT/B HN, a BoNT/Cl HN, a BoNT/D HN, a BoNT/E HN, a BoNT/F HN, and a BoNT/G HN.

As used herein, the term “Clostridial botulinum neurotoxin (BoNT) receptor-binding domain” is synonymous with “HC domain” and “HC”, and means any naturally occurring BoNT receptor binding domain that can execute the cell binding step of the intoxication process, including, e.g., the binding of the BoNT to a BoNT-specific receptor system located on the plasma membrane surface of a target cell. Some aspects of present disclosure relate to modified BoNT receptor binding domains from serotype A2 (BoNT/A2), that enhances the binding of the BoNT/A2 to a cell, e.g., neurons or a BoNT/A2 receptor.

BoNT/A has eight subtypes, BoNT/A1, BoNT/A2, BoNT/A3, BoNT/A4, BoNT/A5, BoNT/A6, BoNT/A7, and BoNT/A8. It is appreciated that when “BoNT/A” is referred to, it encompasses all the subtypes of BoNT/A. In some embodiments, a “modified BoNT/A2 receptor binding domain” comprises novel amino acid substitution mutations described in the present disclosure.

Some aspects of present disclosure relate to modified BoNT receptor binding domains from serotype A2 (BoNT/A2), that enhances the binding of the BoNT/A2 to a cell, e.g., neurons or a BoNT/A2 receptor. Thus, the present disclosure encompasses modified BoNT/A2 receptor binding domain from all and any of the eight subtypes. In some embodiments, a “modified BoNT/A2 receptor binding domain” comprises novel amino acid substitution mutations described in the present disclosure. In some embodiments, the modified receptor binding domain of BoNT/A2 comprises about amino acids 873-1295 of SEQ ID NO: 1. It is to be understood that the border of the BoNT/A2 receptor binding domain fragment may vary by 1-10 amino acids. For example, the modified BoNT/A2 receptor binding domain that may be used for the chimeric toxin may comprise amino acids 863-1296, 864-1296, 865-1296, 866-1296, 867-1296, 868-1296, 869-1296, 870-1296, 871-1296, 872-1296, 873-1296, 874-1296, 875-1296, 876-1296, 877-1296, 878-1296, 879-1296, 880-1296, 881-1296, 882-1296, 883-1296 of SEQ ID NO: 1.

In some aspects, the modified BoNT polypeptide is a chimeric toxin, wherein protease domain and translocation domain are from a serotype selected from the group consisting of A, B, C, D, E, F, G, X or En and combinations thereof and the modified receptor binding domain comprises any one of the BoNT/A1 or BoNT/A2 modified receptor binding domains described herein. In some embodiments the protease and translocation domains may be fused with any one of the modified receptor binding domains of BoNT/A1 or BoNT/A2. In a non-limiting example, a chimeric BoNT/B|A2-R1064W (nomenclature: protease and transmembrane domain from BoNT/B and modified receptor binding from BoNT/A2 with a R1064W modification) may be produced by fusing the protease and transmembrane domain of a BoNT of serotype B with a modified receptor binding domain of BoNT/A1 comprising a arginine to tryptophan mutation at position 1064 of SEQ ID NO: 1. In a non-limiting example, a chimeric BoNT/C|A2-R1064W may be produced by fusing the protease and transmembrane domain of a BoNT of serotype C with a modified receptor binding domain of BoNT/A2 comprising a arginine to tryptophan mutation at position 1065 of SEQ ID NO: 1.

In some embodiments, the modified BoNT polypeptide is a chimeric toxin, wherein protease domain and translocation domain comprise the BoNT/B serotype. The protease and transmembrane domain of BoNT/B comprises about amino acids 1-857 of SEQ ID NO: 2. It is to be understood that the border of the BoNT/B protease and transmembrane domain may vary by 1-10 amino acids. For example, the BoNT/B protease and transmembrane domain that may be used for the chimeric toxin may comprise amino acids 1-847, 1-848, 1-849, 1-850, 1-851, 1-852, 1-853, 1-854, 1-855, 1-856, 1-857, 1-858, 1-859, 1-860, 1-861, 1-862, 1-863, 1-864, 1-865, 1-866, or 1-867 of SEQ ID NO: 2.

In some embodiments, the modified BoNT polypeptide is a chimeric toxin, wherein protease domain and translocation domain comprise the BoNT/C serotype. The protease and transmembrane domain of BoNT/C comprises about amino acids 1-870 of SEQ ID NO: 3. It is to be understood that the border of the BoNT/C protease and transmembrane domain may vary by 1-10 amino acids. For example, the BoNT/C protease and transmembrane domain that may be used for the chimeric toxin may comprise amino acids 1-860, 1-861, 1-862, 1-863, 1-864, 1-865, 1-866, 1-867, 1-868, 1-869, 1-870, 1-871, 1-872, 1-873, 1-874, 1-875, 1-876, 1-877, 1-878, 1-879, or 1-880 of SEQ ID NO: 3.

In some embodiments, the modified BoNT polypeptide is a chimeric toxin, wherein protease domain and translocation domain comprise the BoNT/D serotype. The protease and transmembrane domain of BoNT/D comprises about amino acids 1-862 of SEQ ID NO: 4. It is to be understood that the border of the BoNT/D protease and transmembrane domain may vary by 1-10 amino acids. For example, the BoNT/D protease and transmembrane domain that may be used for the chimeric toxin may comprise amino acids 1-852, 1-853, 1-854, 1-855, 1-856, 1-857, 1-858, 1-859, 1-860, 1-861, 1-862, 1-863, 1-864, 1-865, 1-866, 1-867, 1-868, 1-869, 1-870, 1-871, 1-872 of SEQ ID NO: 4.

In some embodiments, the modified BoNT polypeptide is a chimeric toxin, wherein protease domain and translocation domain comprise the BoNT/E serotype. The protease and transmembrane domain of BoNT/E comprises about amino acids 1-844 of SEQ ID NO: 5. It is to be understood that the border of the BoNT/E protease and transmembrane domain may vary by 1-10 amino acids. For example, the BoNT/E protease and transmembrane domain that may be used for the chimeric toxin may comprise amino acids 1-834, 1-835, 1-836, 1-837, 1-838, 1-839, 1-840, 1-841, 1-842, 1-843, 1-844, 1-845, 1-846, 1-847, 1-848, 1-849, 1-850, 1-851, 1-852, 1-853, 1-854 of SEQ ID NO: 5.

In some embodiments, the modified BoNT polypeptide is a chimeric toxin, wherein protease domain and translocation domain comprise the BoNT/F serotype. The protease and transmembrane domain of BoNT/F comprises about amino acids 1-863 of SEQ ID NO: 6. It is to be understood that the border of the BoNT/F protease and transmembrane domain may vary by 1-10 amino acids. For example, the BoNT/F protease and transmembrane domain that may be used for the chimeric toxin may comprise amino acids 1-853, 1-854, 1-855, 1-856, 1-857, 1-858, 1-859, 1-860, 1-861, 1-862, 1-863, 1-864, 1-865, 1-866, 1-867, 1-868, 1-869, 1-870, 1-871, 1-872, or 1-873 of SEQ ID NO: 6.

In some embodiments, the modified BoNT polypeptide is a chimeric toxin, wherein protease domain and translocation domain comprise the BoNT/G serotype. The protease and transmembrane domain of BoNT/G comprises about amino acids 1-862 of SEQ ID NO: 7. It is to be understood that the border of the BoNT/G protease and transmembrane domain may vary by 1-10 amino acids. For example, the BoNT/G protease and transmembrane domain that may be used for the chimeric toxin may comprise amino acids 1-852, 1-853, 1-854, 1-855, 1-856, 1-857, 1-858, 1-859, 1-860, 1-861, 1-862, 1-863, 1-864, 1-865, 1-866, 1-867, 1-868, 1-869, 1-870, 1-871, or 1-872 of SEQ ID NO: 7.

In some embodiments, the modified BoNT polypeptide is a chimeric toxin, wherein protease domain and translocation domain comprise the BoNT/H serotype. The protease and transmembrane domain of BoNT/H comprises about amino acids 1-858 of SEQ ID NO: 8. It is to be understood that the border of the BoNT/H protease and transmembrane domain may vary by 1-10 amino acids. For example, the BoNT/H protease and transmembrane domain that may be used for the chimeric toxin may comprise amino acids 1-848, 1-849, 1-850, 1-851, 1-852, 1-853, 1-854, 1-855, 1-856, 1-857, 1-858, 1-859, 1-860, 1-861, 1-862, 1-863, 1-864, 1-865, 1-866, 1-867 or 1-868 of SEQ ID NO: 8.

In some embodiments, the modified BoNT polypeptide is a chimeric toxin, wherein protease domain and translocation domain comprise the BoNT/X serotype as described in U.S. application Ser. No. 16/315,698 (incorporated herein by reference). The protease and transmembrane domain of BoNT/X comprises about amino acids 1-889 of SEQ ID NO: 9. It is to be understood that the border of the BoNT/X protease and transmembrane domain may vary by 1-10 amino acids. For example, the BoNT/X protease and transmembrane domain that may be used for the chimeric toxin may comprise amino acids 1-879, 1-880, 1-881, 1-882, 1-883, 1-884, 1-885, 1-886, 1-887, 1-888, 1-889, 1-890, 1-891, 1-892, 1-893, 1-894, 1-895, 1-896, 1-897, 1-898 or 1-899 of SEQ ID NO: 9.

In some embodiments, the modified BoNT polypeptide is a chimeric toxin, wherein protease domain and translocation domain comprise the BoNT/EN serotype as described in U.S. application Ser. No. 16/651,720 (incorporated herein by reference). The protease and transmembrane domain of BoNT/EN comprises about amino acids 1-874 of SEQ ID NO: 10. It is to be understood that the border of the BoNT/EN protease and transmembrane domain may vary by 1-10 amino acids. For example, the BoNT/EN protease and transmembrane domain that may be used for the chimeric toxin may comprise amino acids 1-864, 1-865, 1-866, 1-867, 1-868, 1-869, 1-870, 1-871, 1-872, 1-873, 1-874, 1-875, 1-876, 1-877, 1-878, 1-879, 1-880, 1-881, 1-882, 1-883 or 1-884 of SEQ ID NO: 10.

Some aspects of present disclosure relate to modified BoNT receptor binding domains from serotype A1 (BoNT/A1), that enhances the binding of the BoNT/A1 to a cell, e.g., neurons or a BoNT/A1 receptor. Thus, the present disclosure encompasses modified BoNT/A1 receptor binding domain from all and any of the eight subtypes. In some embodiments, a “modified BoNT/A1 receptor binding domain” comprises novel amino acid substitution mutations described in the present disclosure. In some embodiments, the modified receptor binding domain of BoNT/A1 comprises about amino acids 873-1296 of SEQ ID NO: 11. It is to be understood that the border of the BoNT/A1 receptor binding domain fragment may vary by 1-10 amino acids. For example, the modified BoNT/A1 receptor binding domain that may be used for the chimeric toxin may comprise amino acids 863-1296, 864-1296, 865-1296, 866-1296, 867-1296, 868-1296, 869-1296, 870-1296, 871-1296, 872-1296, 873-1296, 874-1296, 875-1296, 876-1296, 877-1296, 878-1296, 879-1296, 880-1296, 881-1296, 882-1296, 883-1296 of SEQ ID NO: 11.

In some embodiments, the modified receptor binding domain comprises an amino acid substitution at a position corresponding to 1064 in SEQ ID NO: 1. In some embodiments, the modified receptor binding domain comprises an amino acid substitution corresponding to R1064A, R1064W, R1064F, R1064L, R1064Y or R1064H in SEQ ID NO: 1.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 23, and comprises an amino acid substitution corresponding to R1064A, R1064L, R1064Y, R1064W, R1064F or R1064H in SEQ ID NO: 23. In some embodiments, the modified BoNT polypeptide comprises the amino acid sequence of any one of SEQ ID NOs: 24-27 and 62-63, In some embodiments, the modified BoNT polypeptide consists of the amino acid sequence of any one of SEQ ID NOs: 24-27 and 62-63.

In some embodiments, the modified BoNT polypeptide is a full-length BoNT/A2 polypeptide comprising the modified receptor binding domain of BoNT/A2 described herein. In some embodiments, a full length BoNT is a wildtype BoNT. In some embodiments, a full length BoNT is wildtype BoNT that has been modified to comprise a substitution or insertion mutation. A modified full length BoNT may refer to a wildtype sequence that has been modified to comprise a mutation, wherein the mutation is not a deletion (e.g., does not decrease the length of the BoNT). In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1 or SEQ ID NO: 64, and comprises an amino acid substitution corresponding to R1064W, R1064A, R1064L, R1064Y, R1064F or R1064H in SEQ ID NO: 1. In some embodiments, the modified BoNT polypeptide comprises an amino acid sequence of SEQ ID NO: 64 having a substitution mutation corresponding to R1064W, R1064A, R1064L, R1064Y, R1064F or R1064H in SEQ ID NO: 1. In some embodiments, the modified BoNT polypeptide comprises the amino acid sequence of any one of SEQ ID NO: 12-15 and 60-61. In some embodiments, the modified BoNT polypeptide consists of the amino acid sequence of any one of SEQ ID NO: 12-15 and 60-61.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor binding domain of BoNT/A2 comprising an amino acid substitution corresponding to R1064W, R1064A, R1064L, R1064Y, R1064F or R1064H in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 23 and comprises an amino acid substitution corresponding to R1064W, R1064A, R1064L, R1064Y, R1064F or R1064H in SEQ ID NO: 23. In some embodiments, the modified BoNT polypeptide comprises a polypeptide comprising the amino acid sequence of any one of SEQ ID NOs: 35-43, fused to a modified receptor bonding domain of BoNT/A2 comprising the amino acid sequence of any one of SEQ ID NOs: 24-27 and 62-63. In some embodiments, the modified BoNT polypeptide comprises a polypeptide consisting of the amino acid sequence of any one of SEQ ID NOs: 35-43, fused to a modified receptor bonding domain of BoNT/A2 consisting of the amino acid sequence of any one of SEQ ID NOs: 24-27 and 62-63. In some embodiments, the modified BoNT polypeptide comprises the amino acid sequence of any one of SEQ ID NOs: 44-52 having an amino acid substitution corresponding to R1064W, R1064A, R1064L, R1064Y, R1064F or R1064F in SEQ ID NO: 1.

In some embodiments, the modified receptor binding domain comprises an amino acid substitution at a position corresponding to 1064 in SEQ ID NO: 1. In some embodiments, the modified receptor binding domain comprises an amino acid substitution corresponding to R1064W, R1064F, or R1064H in SEQ ID NO: 1.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 23, and comprises an amino acid substitution corresponding to R1064W, R1064F or R1064H in SEQ ID NO: 23. In some embodiments, the modified BoNT polypeptide comprises the amino acid sequence of any one of SEQ ID NOs: 24-26, In some embodiments, the modified BoNT polypeptide consists of the amino acid sequence of any one of SEQ ID NOs: 24-26.

In some embodiments, the modified BoNT polypeptide is a full-length BoNT/A2 polypeptide comprising the modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1 or SEQ ID NO: 64, and comprises an amino acid substitution corresponding to R1064W, R1064F or R1064H in SEQ ID NO: 1. In some embodiments, the modified BoNT polypeptide comprises an amino acid sequence of SEQ ID NO: 64 having a substitution mutation corresponding to R1064W, R1064F or R1064H in SEQ ID NO: 1. In some embodiments, the modified BoNT polypeptide comprises the amino acid sequence of any one of SEQ ID NO: 12-14. In some embodiments, the modified BoNT polypeptide consists of the amino acid sequence of any one of SEQ ID NO: 12-14.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor binding domain of BoNT/A2 comprising an amino acid substitution corresponding to R1064W, R1064A, R1064L, R1064Y, R1064F or R1064H in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 23 and comprises an amino acid substitution corresponding to R1064W, R1064A, R1064L, R1064Y, R1064F or R1064H in SEQ ID NO: 23. In some embodiments, the modified BoNT polypeptide comprises a polypeptide comprising the amino acid sequence of any one of SEQ ID NOs: 35-43, fused to a modified receptor bonding domain of BoNT/A2 comprising the amino acid sequence of any one of SEQ ID NOs: 24-26. In some embodiments, the modified BoNT polypeptide comprises a polypeptide consisting of the amino acid sequence of any one of SEQ ID NOs: 35-43, fused to a modified receptor bonding domain of BoNT/A2 consisting of the amino acid sequence of any one of SEQ ID NOs: 24-26. In some embodiments, the modified BoNT polypeptide comprises the amino acid sequence of any one of SEQ ID NOs: 44-52 having an amino acid substitution corresponding to R1064W, R1064A, R1064L, R1064Y, R1064F or R1064F in SEQ ID NO: 1.

In some embodiments, the modified receptor binding domain comprises an amino acid substitution at a position corresponding to 1294 in SEQ ID NO: 1. In some embodiments, the modified receptor binding domain comprises an amino acid substitution corresponding to S1294R in SEQ ID NO: 1.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 23, and comprises an amino acid substitution corresponding to S1294R in SEQ ID NO: 23. In some embodiments, the modified BoNT polypeptide comprises the amino acid sequence of SEQ ID NO: 28, In some embodiments, the modified BoNT polypeptide consists of the amino acid sequence of SEQ ID NO: 28.

In some embodiments, the modified BoNT polypeptide is a full-length BoNT/A2 polypeptide comprising the modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1 or SEQ ID NO: 64, and comprises an amino acid substitution corresponding to 1294 in SEQ ID NO: 1. In some embodiments, the modified BoNT polypeptide comprises an amino acid sequence of SEQ ID NO: 64 having a substitution mutation corresponding to 1294 in SEQ ID NO: 1. In some embodiments, the modified BoNT polypeptide comprises the amino acid sequence of SEQ ID NO: 16. In some embodiments, the modified BoNT polypeptide consists of the amino acid sequence of SEQ ID NO: 16.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor binding domain of BoNT/A2 comprising an amino acid substitution corresponding to S1294R in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 23 and comprises an amino acid substitution corresponding to 1294R in SEQ ID NO: 23. In some embodiments, the modified BoNT polypeptide comprises a polypeptide comprising the amino acid sequence of any one of SEQ ID NOs: 35-43, fused to a modified receptor bonding domain of BoNT/A2 comprising the amino acid sequence of SEQ ID NO: 28. In some embodiments, the modified BoNT polypeptide comprises a polypeptide consisting of the amino acid sequence of any one of SEQ ID NOs: 35-43, fused to a modified receptor bonding domain of BoNT/A2 consisting of the amino acid sequence of SEQ ID NO: 28. In some embodiments, the modified BoNT polypeptide comprises the amino acid sequence of any one of SEQ ID NOs: 44-52 having an amino acid substitution corresponding to S1294R in SEQ ID NO: 1.

In some embodiments, the modified receptor binding domain comprises an amino acid substitution at a position corresponding to 1295 in SEQ ID NO: 1. In some embodiments, the modified receptor binding domain comprises an amino acid substitution corresponding to S1295P in SEQ ID NO: 1.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 23, and comprises an amino acid substitution corresponding to S1295P in SEQ ID NO: 23. In some embodiments, the modified BoNT polypeptide comprises the amino acid sequence of SEQ ID NO: 29. In some embodiments, the modified BoNT polypeptide consists of the amino acid sequence of SEQ ID NO: 29.

In some embodiments, the modified BoNT polypeptide is a full-length BoNT/A2 polypeptide comprising the modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1 or SEQ ID NO: 64, and comprises an amino acid substitution corresponding to S1295P in SEQ ID NO: 1. In some embodiments, the modified BoNT polypeptide comprises an amino acid sequence of SEQ ID NO: 64 having a substitution mutation corresponding to S1295P in SEQ ID NO: 1. In some embodiments, the modified BoNT polypeptide comprises the amino acid sequence of SEQ ID NO: 17. In some embodiments, the modified BoNT polypeptide consists of the amino acid sequence of SEQ ID NO: 17.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor binding domain of BoNT/A2 comprising an amino acid substitution corresponding to S1295P in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 23 and comprises an amino acid substitution corresponding to S1295P in SEQ ID NO: 23. In some embodiments, the modified BoNT polypeptide comprises a polypeptide comprising the amino acid sequence of any one of SEQ ID NOs: 35-43, fused to a modified receptor bonding domain of BoNT/A2 comprising the amino acid sequence of SEQ ID NO: 29. In some embodiments, the modified BoNT polypeptide comprises a polypeptide consisting of the amino acid sequence of any one of SEQ ID NOs: 35-43, fused to a modified receptor bonding domain of BoNT/A2 consisting of the amino acid sequence of SEQ ID NO: 29. In some embodiments, the modified BoNT polypeptide comprises the amino acid sequence of any one of SEQ ID NOs: 44-52 having an amino acid substitution corresponding to S1295P in SEQ ID NO: 1.

In some embodiments, the modified receptor binding domain comprises an amino acid substitution at a positions corresponding to 1294 and 1295 in SEQ ID NO: 1. In some embodiments, the modified receptor binding domain comprises amino acid substitutions corresponding to S1294R and S1295P in SEQ ID NO: 1.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 23, and comprises amino acid substitutions corresponding to S1294R and S1295P in SEQ ID NO: 23. In some embodiments, the modified BoNT polypeptide comprises the amino acid sequence of SEQ ID NO: 30, In some embodiments, the modified BoNT polypeptide consists of the amino acid sequence of SEQ ID NO: 30.

In some embodiments, the modified BoNT polypeptide is a full-length BoNT/A2 polypeptide comprising the modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1 or SEQ ID NO: 64, and comprises amino acid substitutions corresponding to S1294R and S1295P in SEQ ID NO: 1. In some embodiments, the modified BoNT polypeptide comprises an amino acid sequence of SEQ ID NO: 64 having a substitution mutation substitution corresponding to S1294R and S1295P in SEQ ID NO: 1. In some embodiments, the modified BoNT polypeptide comprises the amino acid sequence of SEQ ID NO: 18. In some embodiments, the modified BoNT polypeptide consists of the amino acid sequence of SEQ ID NO: 18.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor binding domain of BoNT/A2 comprising amino acid substitutions corresponding to S1294R and S1295P in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 23 and comprises an amino acid substitution corresponding to S1294R and S1295P in SEQ ID NO: 23. In some embodiments, the modified BoNT polypeptide comprises a polypeptide comprising the amino acid sequence of any one of SEQ ID NOs: 35-43, fused to a modified receptor bonding domain of BoNT/A2 comprising the amino acid sequence of SEQ ID NO: 30. In some embodiments, the modified BoNT polypeptide comprises a polypeptide consisting of the amino acid sequence of any one of SEQ ID NOs: 35-43, fused to a modified receptor bonding domain of BoNT/A2 consisting of the amino acid sequence of SEQ ID NO: 30. In some embodiments, the modified BoNT polypeptide comprises the amino acid sequence of any one of SEQ ID NOs: 44-52 having amino acid substitutions corresponding to S1294R and S1295P in SEQ ID NO: 1.

In some embodiments, the modified receptor binding domain comprises an amino acid substitution at a position corresponding to 1064, 1294 and 1295 in SEQ ID NO: 1. In some embodiments, the modified BoNT the amino acid substitutions correspond to positions R1064A, R1064L, R1064H, R1064W, R1064F, or R1064Y; and S1294R and S1295P in SEQ ID NO: 1. In some embodiments, the modified receptor binding domain comprises amino acid substitutions corresponding to R1064W, S1294R and S1295P in SEQ ID NO: 1.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 23, and comprises an amino acid substitution corresponding to R1064W, S1294R and S1295P in SEQ ID NO: 23. In some embodiments, the modified BoNT polypeptide comprises the amino acid sequence of SEQ ID NO: 31, In some embodiments, the modified BoNT polypeptide consists of the amino acid sequence of SEQ ID NO: 31.

In some embodiments, the modified BoNT polypeptide is a full-length BoNT/A2 polypeptide comprising the modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1 or SEQ ID NO: 64, and comprises amino acid substitutions corresponding to R1064W, S1294R and S1295P in SEQ ID NO: 1. In some embodiments, the modified BoNT polypeptide comprises an amino acid sequence of SEQ ID NO: 64 having a substitution mutation corresponding to R1064W, S1294R and S1295P in SEQ ID NO: 1. In some embodiments, the modified BoNT polypeptide comprises the amino acid sequence of SEQ ID NO: 19. In some embodiments, the modified BoNT polypeptide consists of the amino acid sequence of SEQ ID NO: 19.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor binding domain of BoNT/A2 comprising amino acid substitutions corresponding to R1064W, S1294R and S1295P in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 23 and comprises amino acid substitutions corresponding to R1064W, S1294R and S1295P in SEQ ID NO: 23. In some embodiments, the modified BoNT polypeptide comprises a polypeptide comprising the amino acid sequence of any one of SEQ ID NOs: 35-43, fused to a modified receptor bonding domain of BoNT/A2 comprising the amino acid sequence of SEQ ID NO: 31. In some embodiments, the modified BoNT polypeptide comprises a polypeptide consisting of the amino acid sequence of any one of SEQ ID NOs: 35-43, fused to a modified receptor bonding domain of BoNT/A2 consisting of the amino acid sequence of SEQ ID NO: 31. In some embodiments, the modified BoNT polypeptide comprises the amino acid sequence of any one of SEQ ID NOs: 44-52 having amino acid substitutions corresponding to R1064W, S1294R and S1295P in SEQ ID NO: 1.

In some embodiments, the modified receptor binding domain comprises amino acid substitutions corresponding to R1064F, S1294R and S1295P in SEQ ID NO: 1.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 23, and comprises amino acid substitutions corresponding to R1064F, S1294R and S1295P in SEQ ID NO: 23. In some embodiments, the modified BoNT polypeptide comprises the amino acid sequence of SEQ ID NO: 32, In some embodiments, the modified BoNT polypeptide consists of the amino acid sequence of SEQ ID NO: 32.

In some embodiments, the modified BoNT polypeptide is a full-length BoNT/A2 polypeptide comprising the modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1 or SEQ ID NO: 64, and comprises amino acid substitutions corresponding to R1064F, S1294R and S1295P in SEQ ID NO: 1. In some embodiments, the modified BoNT polypeptide comprises an amino acid sequence of SEQ ID NO: 64 having a substitution mutation corresponding to R1064F, S1294R and S1295P in SEQ ID NO: 1. In some embodiments, the modified BoNT polypeptide comprises the amino acid sequence of SEQ ID NO: 20. In some embodiments, the modified BoNT polypeptide consists of the amino acid sequence of SEQ ID NO: 20.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor binding domain of BoNT/A2 comprising amino acid substitutions corresponding to R1064F, S1294R and S1295P in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 23 and comprises amino acid substitutions corresponding to R1064F, S1294R and S1295P in SEQ ID NO: 23. In some embodiments, the modified BoNT polypeptide comprises a polypeptide comprising the amino acid sequence of any one of SEQ ID NOs: 35-43, fused to a modified receptor bonding domain of BoNT/A2 comprising the amino acid sequence of SEQ ID NO: 32. In some embodiments, the modified BoNT polypeptide comprises a polypeptide consisting of the amino acid sequence of any one of SEQ ID NOs: 35-43, fused to a modified receptor bonding domain of BoNT/A2 consisting of the amino acid sequence of SEQ ID NO: 32. In some embodiments, the modified BoNT polypeptide comprises the amino acid sequence of any one of SEQ ID NOs: 44-52 having amino acid substitutions corresponding to R1064F, S1294R and S1295P in SEQ ID NO: 1.

In some embodiments, the modified receptor binding domain comprises amino acid substitutions corresponding to R1064H, S1294R and S1295P in SEQ ID NO: 1.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 23, and comprises amino acid substitutions corresponding to R1064H, S1294R and S1295P in SEQ ID NO: 23.

In some embodiments, the modified BoNT polypeptide is a full-length BoNT/A2 polypeptide comprising the modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1 or SEQ ID NO: 64, and comprises amino acid substitutions corresponding to R1064H, S1294R and S1295P in SEQ ID NO: 1. In some embodiments, the modified BoNT polypeptide comprises an amino acid sequence of SEQ ID NO: 64 having a substitution mutation corresponding to R1064H, S1294R and S1295P in SEQ ID NO: 1.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor binding domain of BoNT/A2 comprising amino acid substitutions corresponding to R1064H, S1294R and S1295P in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 23 and comprises amino acid substitutions corresponding to R1064H, S1294R and S1295P in SEQ ID NO: 23.

In some embodiments, the modified receptor binding domain comprises amino acid substitutions corresponding to R1064L, S1294R and S1295P in SEQ ID NO: 1.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 23, and comprises amino acid substitutions corresponding to R1064L, S1294R and S1295P in SEQ ID NO: 23.

In some embodiments, the modified BoNT polypeptide is a full-length BoNT/A2 polypeptide comprising the modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1 or SEQ ID NO: 64, and comprises amino acid substitutions corresponding to R1064L, S1294R and S1295P in SEQ ID NO: 1. In some embodiments, the modified BoNT polypeptide comprises an amino acid sequence of SEQ ID NO: 64 having a substitution mutation corresponding to R1064L, S1294R and S1295P in SEQ ID NO: 1.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor binding domain of BoNT/A2 comprising amino acid substitutions corresponding to R1064L, S1294R and S1295P in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 23 and comprises amino acid substitutions corresponding to R1064L, S1294R and S1295P in SEQ ID NO: 23.

In some embodiments, the modified receptor binding domain comprises amino acid substitutions corresponding to R1064Y, S1294R and S1295P in SEQ ID NO: 1.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 23, and comprises amino acid substitutions corresponding to R1064Y, S1294R and S1295P in SEQ ID NO: 23.

In some embodiments, the modified BoNT polypeptide is a full-length BoNT/A2 polypeptide comprising the modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1 or SEQ ID NO: 64, and comprises amino acid substitutions corresponding to R1064Y, S1294R and S1295P in SEQ ID NO: 1. In some embodiments, the modified BoNT polypeptide comprises an amino acid sequence of SEQ ID NO: 64 having a substitution mutation corresponding to R1064Y, S1294R and S1295P in SEQ ID NO: 1.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor binding domain of BoNT/A2 comprising amino acid substitutions corresponding to R1064Y, S1294R and S1295P in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 23 and comprises amino acid substitutions corresponding to R1064Y, S1294R and S1295P in SEQ ID NO: 23.

In some embodiments, the modified receptor binding domain comprises amino acid substitutions corresponding to R1064A, S1294R and S1295P in SEQ ID NO: 1.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 23, and comprises amino acid substitutions corresponding to R1064A, S1294R and S1295P in SEQ ID NO: 23.

In some embodiments, the modified BoNT polypeptide is a full-length BoNT/A2 polypeptide comprising the modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1 or SEQ ID NO: 64, and comprises amino acid substitutions corresponding to R1064A, S1294R and S1295P in SEQ ID NO: 1. In some embodiments, the modified BoNT polypeptide comprises an amino acid sequence of SEQ ID NO: 64 having a substitution mutation corresponding to R1064A, S1294R and S1295P in SEQ ID NO: 1.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor binding domain of BoNT/A2 comprising amino acid substitutions corresponding to R1064A, S1294R and S1295P in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 35-43, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 23 and comprises amino acid substitutions corresponding to R1064A, S1294R and S1295P in SEQ ID NO: 23.

In some aspects, this application discloses a modified BoNT comprising a BoNT/A2 receptor binding domain that comprises an amino acid substitution which decreases BoNT binding to a receptor. For example, in some embodiments, the modified BoNT comprises an amino acid substitution corresponding to P1063T in SEQ ID NO: 1. In another example, in some embodiments, the modified BoNT comprises amino acid substitutions corresponding to P1063T and R1064H in SEQ ID NO: 1.

In some embodiments, the modified BoNTs described herein do not comprise amino acid substitutions that decrease receptor binding. For example, in some embodiments, the modified BoNT does not comprise an amino acid substitution corresponding to P1063T or in SEQ ID NO: 1. In another example, in some embodiments, the modified BoNT does not comprises amino acid substitutions corresponding to P1063T and R1064H in SEQ ID NO: 1.

In some embodiments, this disclose describes a modified BoNT/A2 wildtype protein (e.g., as described herein), wherein the wildtype cleavage sequence of the BoNT/A2 is replaced with thrombin. In some embodiments, the modified BoNT polypeptide comprises a polypeptide comprises an amino acid sequence that is at least 70% (e.g., at least 70%, at least 75%, at least 80%, at least 85%, at least 90%, at least 95%, or at least 99%) identical to SEQ ID NO: 64. In some embodiments, the modified BoNT polypeptide comprises SEQ ID NO: 64.

In some embodiments, the modified BoNT polypeptide comprises a polypeptide comprises an amino acid sequence that is at least 70% (e.g., at least 70%, at least 75%, at least 80%, at least 85%, at least 90%, at least 95%, or at least 99%) identical to SEQ ID NO: 65. In some embodiments, the modified BoNT polypeptide comprises SEQ ID NO: 65.

### BoNT Linkers

Wildtype full length BoNTs comprise a linker region between LC and HN that needs to be separated by proteases in order to release LC upon entry into the cytosol. This process is known as “activation.” Activated LC-HN remains connected by a disulfide bond before reaching the cytosol. To facilitate the activation step, in some embodiments, the wildtype linker sequence may be replaced by a modified linker region (e.g., a thrombin cleavage site), and a linker region from a different BoNT.

In some embodiments, the modified BoNT polypeptides comprises a modified linker region, wherein the linker region comprises a specific protease cleavage site. A “specific protease cleavage site,” as used herein, refers to a recognition and cleavage site for a specification protease, as opposed to a sequence that is recognized and cleavage by more than one non-specific proteases. Such specific proteases include, without limitation: thrombin, TEV, PreScission, Factor Xa, MMP-12, MMP-13, MMP-17, MMP-20, Granzyme-B, and Enterokinase.

In some embodiments, the cleavage site of the specific proteases may be added to the linker region of a modified BoNT polypeptide described herein via insertion or replacement of the existing amino acids in the linker region (e.g. replace amino acids 430-454 of modified BoNT polypeptides comprising the LC-HN domain of BoNT/A2). The sequences of the specific protease cleavage sites sequences are also provided: LVPR|GS (thrombin, SEQ ID NO: 53), ENLYFQ|G (TEV, SEQ ID NO: 54), LEVLFQ|GP (PreScission, SEQ ID NO: 55), IEGR| or IDGR| (Factor Xa, SEQ ID NOs: 56-57), DDDDK| (Enterokinase, SEQ ID NO: 58) and AHREQIGG| (SUMO protease, SEQ ID NO: 59). “|” indicates where cleavage occurs.

### Polynucleotides and BoNT Production

The modified BoNT polypeptides of the present disclosure (e.g., without limitation, polypeptides comprising amino acid sequence of any of SEQ ID NOs: 12-22, 24-34 and 60-63), will generally be produced by expression from recombinant nucleic acids in appropriate cells (e.g., E. coli, or insect cells) and isolated. The nucleic acids encoding the polypeptides described herein may be obtained, and the nucleotide sequence of the nucleic acids determined, by any method known in the art.

Further provided herein are isolated and/or recombinant nucleic acids encoding any of the modified BoNT polypeptides disclosed herein. The nucleic acids encoding the isolated polypeptide fragments of the present disclosure, may be DNA or RNA, double-stranded or single stranded. In certain aspects, the subject nucleic acids encoding the isolated polypeptide fragments are further understood to include nucleic acids encoding polypeptides that are variants of any of the modified BoNT polypeptides described herein.

Variant nucleotide sequences include sequences that differ by one or more nucleotide substitutions, additions or deletions, such as allelic variants. In some embodiments, the isolated nucleic acid molecule of the present disclosure comprising a polynucleotide encoding a polypeptide comprising an amino acid sequence that has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identity of any of SEQ ID NOs: 12-22, 24-34 and 60-63. In some embodiments, the isolated nucleic acid molecule of the present disclosure comprising a polynucleotide encoding a polypeptide comprising an amino acid sequence that has 85%, 86%, 87%, 88%, 89%, 90%, 91%, 92%, 93%, 94%, 95%, 96%, 97%, 98%, 99%, or 100% identity of any of SEQ ID NOs: 12-22, 24-34, and 60-63.

In some embodiments, the nucleic acid is comprised within a vector, such as an expression vector. In some embodiments, the vector comprises a promoter operably linked to the nucleic acid.

A variety of promoters can be used for expression of the polypeptides described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter. Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)].

Other systems include FK506 dimer, VP16 or p65 using astradiol, RU486, diphenol murislerone, or rapamycin. Inducible systems are available from Invitrogen, Clontech and Ariad. Regulatable promoters that include a repressor with the operon can be used. In one embodiment, the lac repressor from Escherichia coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used (Yao et al., Human Gene Therapy; Gossen et al., Natl. Acad. Sci. USA, 89:5547-5551 (1992); Shockett et al., Proc. Natl. Acad. Sci. USA, 92:6522-6526 (1995)).

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

An expression vector comprising the nucleic acid can be transferred to a host cell by conventional techniques (e.g., electroporation, liposomal transfection, and calcium phosphate precipitation) and the transfected cells are then cultured by conventional techniques to produce the polypeptides described herein. In some embodiments, the expression of the polypeptides described herein is regulated by a constitutive, an inducible or a tissue-specific promoter.

The host cells used to express the isolated polypeptides described herein may be either bacterial cells such as Escherichia coli, or, preferably, eukaryotic cells. In particular, mammalian cells, such as Chinese hamster ovary cells (CHO), in conjunction with a vector such as the major intermediate early gene promoter element from human cytomegalovirus is an effective expression system for immunoglobulins (Foecking et al. (1986) “Powerful And Versatile Enhancer-Promoter Unit For Mammalian Expression Vectors,” Gene 45:101-106; Cockett et al. (1990) “High Level Expression Of Tissue Inhibitor Of Metalloproteinases In Chinese Hamster Ovary Cells Using Glutamine Synthetase Gene Amplification,” Biotechnology 8:662-667). A variety of host-expression vector systems may be utilized to express the isolated polypeptides described herein. Such host-expression systems represent vehicles by which the coding sequences of the isolated polypeptides described herein may be produced and subsequently purified, but also represent cells which may, when transformed or transfected with the appropriate nucleotide coding sequences, express the isolated polypeptides described herein in situ. These include, but are not limited to, microorganisms such as bacteria (e.g., E. coli and B. subtilis) transformed with recombinant bacteriophage DNA, plasmid DNA or cosmid DNA expression vectors containing coding sequences for the isolated polypeptides described herein; yeast (e.g., Saccharomyces pichia) transformed with recombinant yeast expression vectors containing sequences encoding the isolated polypeptides described herein; insect cell systems infected with recombinant virus expression vectors (e.g., baclovirus) containing the sequences encoding the isolated polypeptides described herein; plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing sequences encoding the isolated polypeptides described herein; or mammalian cell systems (e.g., COS, CHO, BHK, 293, 293T, 3T3 cells, lymphotic cells (see U.S. Pat. No. 5,807,715), Per C.6 cells (human retinal cells developed by Crucell) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the polypeptides being expressed. For example, when a large quantity of such a protein is to be produced, for the generation of pharmaceutical compositions of polypeptides described herein, vectors which direct the expression of high levels of fusion protein products that are readily purified may be desirable. Such vectors include, but are not limited, to the E. coli expression vector pUR278 (Ruther et al. (1983) “Easy Identification Of cDNA Clones,” EMBO J. 2:1791-1794), in which the coding sequence may be ligated individually into the vector in frame with the lac Z coding region so that a fusion protein is produced; pIN vectors (Inouye et al. (1985) “Up-Promoter Mutations In The lpp Gene Of Escherichia coli,” Nucleic Acids Res. 13:3101-3110; Van Heeke et al. (1989) “Expression Of Human Asparagine Synthetase In Escherichia coli,” J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione.

The pGEX vectors are designed to include thrombin or factor Xa protease cleavage sites so that the cloned target gene product can be released from the GST moiety. In an insect system, Autographa californica nuclear polyhedrosis virus (AcNPV) is used as a vector to express foreign genes. The virus grows in Spodoptera frugiperda cells. The coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts (e.g., see Logan et al. (1984) “Adenovirus Tripartite Leader Sequence Enhances Translation Of mRNAs Late After Infection,” Proc. Natl. Acad. Sci. USA 81:3655-3659). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic.

The efficiency of expression may be enhanced by the inclusion of appropriate transcription enhancer elements, transcription terminators, etc. (see Bitter et al. (1987) “Expression And Secretion Vectors For Yeast,” Methods in Enzymol. 153:516-544). In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. For example, in certain embodiments, the polypeptides described herein may be expressed as a single gene product (e.g., as a single polypeptide chain, i.e., as a polyprotein precursor), requiring proteolytic cleavage by native or recombinant cellular mechanisms to form separate polypeptides described herein.

The disclosure thus encompasses engineering a nucleic acid sequence to encode a polyprotein precursor molecule comprising the polypeptides (i.e., modified BoNTs) described herein, which includes coding sequences capable of directing post translational cleavage of said polyprotein precursor. Post-translational cleavage of the polyprotein precursor results in the polypeptides described herein. The post translational cleavage of the precursor molecule comprising the polypeptides described herein may occur in vivo (i.e., within the host cell by native or recombinant cell systems/mechanisms, e.g. furin cleavage at an appropriate site) or may occur in vitro (e.g. incubation of said polypeptide chain in a composition comprising proteases or peptidases of known activity and/or in a composition comprising conditions or reagents known to foster the desired proteolytic action).

Purification and modification of recombinant proteins is well known in the art such that the design of the polyprotein precursor could include a number of embodiments readily appreciated by a skilled worker. Any known proteases or peptidases known in the art can be used for the described modification of the precursor molecule, e.g., thrombin or factor Xa (Nagai et al. (1985) “Oxygen Binding Properties Of Human Mutant Hemoglobins Synthesized In Escherichia coli,” Proc. Nat. Acad. Sci. USA 82:7252-7255, and reviewed in Jenny et al. (2003) “A Critical Review Of The Methods For Cleavage Of Fusion Proteins With Thrombin And Factor Xa,” Protein Expr. Purif. 31:1-11, each of which is incorporated by reference herein in its entirety)), enterokinase (Collins-Racie et al. (1995) “Production Of Recombinant Bovine Enterokinase Catalytic Subunit In Escherichia coli Using The Novel Secretory Fusion Partner DsbA,” Biotechnology 13:982-987 hereby incorporated by reference herein in its entirety)), furin, and AcTEV (Parks et al. (1994) “Release Of Proteins And Peptides From Fusion Proteins Using A Recombinant Plant Virus Proteinase,” Anal. Biochem. 216:413-417 hereby incorporated by reference herein in its entirety)) and the Foot and Mouth Disease Virus Protease C3.

Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 293T, 3T3, WI38, BT483, Hs578T, HTB2, BT20 and T47D, CRL7030 and Hs578Bst.

For long-term, high-yield production of recombinant proteins, stable expression is preferred. For example, cell lines which stably express polypeptides described herein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the polypeptides described herein. Such engineered cell lines may be particularly useful in screening and evaluation of polypeptides that interact directly or indirectly with the polypeptides described herein.

A number of selection systems may be used, including but not limited to the herpes simplex virus thymidine kinase (Wigler et al. (1977) “Transfer Of Purified Herpes Virus Thymidine Kinase Gene To Cultured Mouse Cells,” Cell 11: 223-232), hypoxanthine-guanine phosphoribosyltransferase (Szybalska et al. (1992) “Use Of The HPRT Gene And The HAT Selection Technique In DNA-Mediated Transformation Of Mammalian Cells First Steps Toward Developing Hybridoma Techniques And Gene Therapy,” Bioessays 14: 495-500), and adenine phosphoribosyltransferase (Lowy et al. (1980) “Isolation Of Transforming DNA: Cloning The Hamster aprt Gene,” Cell 22: 817-823) genes can be employed in tk-, hgprt- or aprt-cells, respectively. Also, antimetabolite resistance can be used as the basis of selection for the following genes: dhfr, which confers resistance to methotrexate (Wigler et al. (1980) “Transformation Of Mammalian Cells With An Amplifiable Dominant-Acting Gene,” Proc. Natl. Acad. Sci. USA 77:3567-3570; O'Hare et al. (1981) “Transformation Of Mouse Fibroblasts To Methotrexate Resistance By A Recombinant Plasmid Expressing A Prokaryotic Dihydrofolate Reductase,” Proc. Natl. Acad. Sci. USA 78: 1527-1531); gpt, which confers resistance to mycophenolic acid (Mulligan et al. (1981) “Selection For Animal Cells That Express The Escherichia coli Gene Coding For Xanthine-Guanine Phosphoribosyltransferase,” Proc. Natl. Acad. Sci. USA 78: 2072-2076); neo, which confers resistance to the aminoglycoside G-418 (Tolstoshev (1993) “Gene Therapy, Concepts, Current Trials And Future Directions,” Ann. Rev. Pharmacol. Toxicol. 32:573-596; Mulligan (1993) “The Basic Science Of Gene Therapy,” Science 260:926-932; and Morgan et al. (1993) “Human Gene Therapy,” Ann. Rev. Biochem. 62:191-217) and hygro, which confers resistance to hygromycin (Santerre et al. (1984) “Expression Of Prokaryotic Genes For Hygromycin B And G418 Resistance As Dominant-Selection Markers In Mouse L Cells,” Gene 30:147-156). Methods commonly known in the art of recombinant DNA technology which can be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al. (1981) “A New Dominant Hybrid Selective Marker For Higher Eukaryotic Cells,” J. Mol. Biol. 150:1-14.

The expression levels of polypeptides described herein can be increased by vector amplification (for a review, see Bebbington and Hentschel, The use of vectors based on gene amplification for the expression of cloned genes in mammalian cells in DNA cloning, Vol. 3 (Academic Press, New York, 1987). When a marker in the vector system expressing a polypeptide described herein is amplifiable, increase in the level of inhibitor present in culture of host cell will increase the number of copies of the marker gene. Since the amplified region is associated with the nucleotide sequence of a polypeptide described herein or a polypeptide described herein, production of the polypeptide will also increase (Crouse et al. (1983) “Expression And Amplification Of Engineered Mouse Dihydrofolate Reductase Minigenes,” Mol. Cell. Biol. 3:257-266).

Once a polypeptide described herein has been recombinantly expressed, it may be purified by any method known in the art for purification of polypeptides, polyproteins or antibodies (e.g., analogous to antibody purification schemes based on antigen selectivity) for example, by chromatography (e.g., ion exchange, affinity, particularly by affinity for the specific antigen (optionally after Protein A selection where the polypeptide comprises an Fc domain (or portion thereof)), and sizing column chromatography), centrifugation, differential solubility, or by any other standard technique for the purification of polypeptides or antibodies. Other aspects of the present disclosure relate to a cell comprising a nucleic acid described herein or a vector described herein.

The cell may be a prokaryotic or eukaryotic cell. In some embodiments, the cell in a mammalian cell. Exemplary cell types are described herein. Other aspects of the present disclosure related to a cell expressing the modified BoNT polypeptides described herein. The cell may be a prokaryotic or eukaryotic cell. In some embodiments, the cell in a mammalian cell. Exemplary cell types are described herein. The cell can be for propagation of the nucleic acid or for expression of the nucleic acid, or both. Such cells include, without limitation, prokaryotic cells including, without limitation, strains of aerobic, microaerophilic, capnophilic, facultative, anaerobic, gram-negative and gram-positive bacterial cells such as those derived from, e.g., Escherichia coli, Bacillus subtilis, Bacillus licheniformis, Bacteroides fragilis, Clostridia perfringens, Clostridia difficile, Caulobacter crescentus, Lactococcus lactis, Methylobacterium extorquens, Neisseria meningirulls, Neisseria meningitidis, Pseudomonas fluorescens and Salmonella typhimurium; and eukaryotic cells including, without limitation, yeast strains, such as, e.g., those derived from Pichia pastoris, Pichia methanolica, Pichia angusta, Schizosaccharomyces pombe, Saccharomyces cerevisiae and Yarrowia lipolytica; insect cells and cell lines derived from insects, such as, e.g., those derived from Spodoptera frugiperda, Trichoplusia ni, Drosophila melanogaster and Manduca sexta; and mammalian cells and cell lines derived from mammalian cells, such as, e.g., those derived from mouse, rat, hamster, porcine, bovine, equine, primate and human. Cell lines may be obtained from the American Type Culture Collection, European Collection of Cell Cultures and the German Collection of Microorganisms and Cell Cultures. Non-limiting examples of specific protocols for selecting, making and using an appropriate cell line are described in e.g., INSECT CELL CULTURE ENGINEERING (Mattheus F. A. Goosen et al. eds., Marcel Dekker, 1993); INSECT CELL CULTURES: FUNDAMENTAL AND APPLIED ASPECTS (J. M. Vlak et al. eds., Kluwer Academic Publishers, 1996); Maureen A. Harrison & Ian F. Rae, GENERAL TECHNIQUES OF CELL CULTURE (Cambridge University Press, 1997); CELL AND TISSUE CULTURE: LABORATORY PROCEDURES (Alan Doyle et al eds., John Wiley and Sons, 1998); R. Ian Freshney, CULTURE OF ANIMAL CELLS: A MANUAL OF BASIC TECHNIQUE (Wiley-Liss, 4.sup.th ed. 2000); ANIMAL CELL CULTURE: A PRACTICAL APPROACH (John R. W. Masters ed., Oxford University Press, 3.sup.rd ed. 2000); MOLECULAR CLONING A LABORATORY MANUAL, supra, (2001); BASIC CELL CULTURE: A PRACTICAL APPROACH (John M. Davis, Oxford Press, 2.sup.nd ed. 2002); and CURRENT PROTOCOLS IN MOLECULAR BIOLOGY, supra, (2004).

These protocols are routine procedures within the scope of one skilled in the art and from the teaching herein. Yet other aspects of the present disclosure relate to a method of producing a polypeptide described herein, the method comprising obtaining a cell described herein and expressing nucleic acid described herein in said cell. In some embodiments, the method further comprises isolating and purifying a polypeptide described herein.

In some embodiments, botulinum neurotoxin can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive.

The bacterial strains that make botulinum toxin serotypes A and G possess endogenous proteases and serotypes A and G can therefore be recovered from bacterial cultures in predominantly their active form. In contrast, botulinum toxin serotypes Ci, D and E are synthesized by non-proteolytic strains and are therefore typically unactivated when recovered from culture. Serotypes B and F are produced by both proteolytic and non-proteolytic strains and therefore can be recovered in either the active or inactive form. The proteolytic strains that produce, for example, the botulinum toxin type A serotype may only cleave a portion of the toxin produced.

The exact proportion of nicked to un-nicked molecules depends on the length of incubation and the temperature of the culture. Therefore, a certain percentage of a preparation of, for example, the botulinum toxin type A toxin may be inactive. In one embodiment, the neurotoxin of the present disclosure is in an active state. In one embodiment, the neurotoxin is in an inactive state. In one embodiment, a combination of active and inactive neurotoxin is envisioned.

### Pharmaceutical Compositions

Thus, the present disclosure also contemplates pharmaceutically compositions comprising the modified BoNTs or the chimeric molecules of the present disclosure. As it may also become clear later in the present disclosure, the pharmaceutical composition of the present disclosure, may further comprise other therapeutic agents suitable for the specific disease such composition is designed to treat. In some embodiments, the pharmaceutically composition of the present disclosure further comprises pharmaceutically-acceptable carriers.

The term “pharmaceutically-acceptable carrier”, as used herein, means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the polypeptide from one site (e.g., the delivery site) of the body, to another site (e.g., organ, tissue or portion of the body).

A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethylcellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier”, “pharmaceutically acceptable excipient” or the like are used interchangeably herein. In some embodiments, a modified BoNT polypeptide of the present disclosure in a composition is administered by injection, by means of a catheter, by means of a suppository, or by means of an implant, the implant being of a porous, non-porous, or gelatinous material, including a membrane, such as a sialastic membrane, or a fiber.

### Administration

Typically, when administering the composition, materials to which the polypeptide of the disclosure does not absorb are used. In other embodiments, the modified BoNT polypeptides of the present disclosure are delivered in a controlled release system. Such compositions and methods for administration are provides in U.S. Patent publication No. 2007/0020295, the contents of which are herein incorporated by reference. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

The modified BoNT polypeptides of the present disclosure can be administered as pharmaceutical compositions comprising a therapeutically effective amount of a binding agent and one or more pharmaceutically compatible ingredients. In typical embodiments, the pharmaceutical composition is formulated in accordance with routine procedures as a pharmaceutical composition adapted for intravenous or subcutaneous administration to a subject, e.g., a human being.

Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration. A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein.

The polypeptides of the present disclosure can be entrapped in ‘stabilized plasmid-lipid particles’ (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757. The pharmaceutical compositions of the present disclosure may be administered or packaged as a unit dose, for example.

The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle. In some embodiments, the modified BoNT polypeptides described herein may be conjugated to a therapeutic moiety, e.g., an antibiotic. Techniques for conjugating such therapeutic moieties to polypeptides, including e.g., Fc domains, are well known; see, e.g., Amon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Hellstrom et al., “Antibodies For Drug Delivery”, in Controlled Drug Delivery (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in Monoclonal Antibodies '84: Biological And Clinical Applications, Pinchera et al. (eds.), 1985, pp. 475-506); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), 1985, pp. 303-16, Academic Press; and Thorpe et al. (1982) “The Preparation And Cytotoxic Properties Of Antibody-Toxin Conjugates,” Immunol. Rev., 62:119-158. Further, the pharmaceutical composition can be provided as a pharmaceutical kit comprising (a) a container containing a polypeptide of the disclosure in lyophilized form and (b) a second container containing a pharmaceutically acceptable diluent (e.g., sterile water) for injection. The pharmaceutically acceptable diluent can be used for reconstitution or dilution of the lyophilized polypeptide of the disclosure. Optionally associated with such container(s) can be a notice in the form prescribed by a governmental agency regulating the manufacture, use or sale of pharmaceuticals or biological products, which notice reflects approval by the agency of manufacture, use or sale for human administration. In another aspect, an article of manufacture containing materials useful for the treatment of the diseases described above is included. In some embodiments, the article of manufacture comprises a container and a label.

Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is an isolated polypeptide of the disclosure. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Methods of Treatment

The modified BoNT polypeptides, the chimeric molecules, and the pharmaceutical compositions of the present disclosure may be used for the treatment of conditions associated with unwanted neuronal activities. Thus, further provided herein are methods of treating a condition associated with unwanted neuronal activity, the method comprising administering a therapeutically effective amount of the modified BoNT polypeptide, the chimeric molecule, or the pharmaceutical composition described herein to thereby treat the condition. In some embodiments, the modified BoNT polypeptides, the chimeric molecules, and the pharmaceutic compositions of the present disclosure contact one or more neuron(s) exhibiting unwanted neuronal activity,

Condition typically treated with a neurotoxin (e.g., skeletal muscle conditions, smooth muscle conditions, glandular conditions, a neuromuscular disorder, an autonomic disorder, pain, or an aesthetic/cosmetic condition) are associated with unwanted neuronal activity, as determined by the skilled practitioner. Administration is by a route that contacts an effective amount of the composition to neurons exhibiting the unwanted activity. In some embodiments, the condition may be associated with overactive neurons or glands. Specific conditions envisioned for treatment by the methods discussed herein include, without limitation, spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions as well as other secretory disorders, pain from muscle spasms, headache pain. In addition, the present disclosure can be used to treat dermatological or aesthetic/cosmetic conditions, for example, reduction of brow furrows, reduction of skin wrinkles.

The present disclosure can also be used in the treatment of sports injuries. Borodic U.S. Pat. No. 5,053,005 discloses methods for treating juvenile spinal curvature, i.e. scoliosis, using botulinum type A. The disclosure of Borodic is incorporated in its entirety herein by reference. In one embodiment, using substantially similar methods as disclosed by Borodic, a modified neurotoxin can be administered to a mammal, preferably a human, to treat spinal curvature. In some embodiments, a modified neurotoxin comprising botulinum type A-E comprising a BoNT/A2 modified receptor binding domain as described herein and a leucine-based motif fused to the carboxyl terminal of its light chain is administered to the mammal, preferably a human, to treat spinal curvature.

In addition, the modified neurotoxin can be administered to treat other neuromuscular disorders using well known techniques that are commonly performed with botulinum type A. For example, the present disclosure can be used to treat pain, for example, headache pain, pain from muscle spasms and various forms of inflammatory pain. For example, Aoki U.S. Pat. No. 5,721,215 and Aoki U.S. Pat. No. 6,113,915 disclose methods of using botulinum toxin type A for treating pain. The disclosure of these two patents is incorporated in its entirety herein by reference.

Autonomic nervous system disorders can also be treated with a modified neurotoxin. For example, glandular malfunctioning is an autonomic nervous system disorder. Glandular malfunctioning includes excessive sweating and excessive salivation. Respiratory malfunctioning is another example of an autonomic nervous system disorder. Respiratory malfunctioning includes chronic obstructive pulmonary disease and asthma. Sanders et al. disclose methods for treating the autonomic nervous system; for example, treating autonomic nervous system disorders such as excessive sweating, excessive salivation, asthma, etc., using naturally existing botulinum toxins. The disclosure of Sander et al. is incorporated in its entirety by reference herein.

In one embodiment, substantially similar methods to that of Sanders et al. can be employed, but using a modified neurotoxin, to treat autonomic nervous system disorders such as the ones discussed above. For example, a modified neurotoxin can be locally applied to the nasal cavity of the mammal in an amount sufficient to degenerate cholinergic neurons of the autonomic nervous system that control the mucous secretion in the nasal cavity. Pain that can be treated by a modified neurotoxin includes pain caused by muscle tension, or spasm, or pain that is not associated with muscle spasm. For example, Binder in U.S. Pat. No. 5,714,468 discloses that headache caused by vascular disturbances, muscular tension, neuralgia and neuropathy can be treated with a naturally occurring botulinum toxin, for example botulinum type A. The disclosures of Binder are incorporated in its entirety herein by reference.

In one embodiment, substantially similar methods to that of Binder can be employed, but using a modified neurotoxin, to treat headache, especially the ones caused by vascular disturbances, muscular tension, neuralgia and neuropathy. Pain caused by muscle spasm can also be treated by an administration of a modified neurotoxin. For example, a botulinum type E fused with a leucine-based motif, preferably at the carboxyl terminal of the botulinum type E light chain, can be administered intramuscularly at the pain/spasm location to alleviate pain. Furthermore, a modified neurotoxin can be administered to a mammal to treat pain that is not associated with a muscular disorder, such as spasm.

In one broad embodiment, methods of the present disclosure to treat non-spasm related pain include central administration or peripheral administration of the modified neurotoxin. For example, Foster et al. in U.S. Pat. No. 5,989,545 discloses that a botulinum toxin conjugated with a targeting moiety can be administered centrally (intrathecally) to alleviate pain. The disclosures of Foster et al. are incorporated in its entirety by reference herein.

In one embodiment, substantially similar methods to that of Foster et al. can be employed, but using the compositions described herein to treat pain. The pain to be treated can be an acute pain or chronic pain. An acute or chronic pain that is not associated with a muscle spasm can also be alleviated with a local, peripheral administration of the modified neurotoxin to an actual or a perceived pain location on the mammal.

In one embodiment, the modified neurotoxin is administered subcutaneously at or near the location of pain, for example, at or near a cut. In some embodiments, the modified neurotoxin is administered intramuscularly at or near the location of pain, for example, at or near a bruise location on the mammal. In some embodiments, the modified BoNT polypeptide is injected directly into a joint of a mammal, for treating or alleviating pain caused by arthritic conditions. Also, frequent repeated injection or infusion of the modified neurotoxin to a peripheral pain location is within the scope of the present disclosure. Routes of administration for such methods are known in the art and easily adapted to the methods described herein by the skilled practitioner (e.g., see for example, Harrison's Principles of Internal Medicine (1998), edited by Anthony Fauci et al., 14.sup.th edition, published by McGraw Hill).

By way of non-limiting example, the treatment of a neuromuscular disorder can comprise a step of locally administering an effective amount of the molecule to a muscle or a group of muscles, the treatment of an autonomic disorder can comprise a step of locally administering an effective of the molecule to a gland or glands, and the treatment of pain can comprise a step of administering an effective amount of the molecule the site of the pain. In addition, the treatment of pain can comprise a step of administering an effective amount of a modified neurotoxin to the spinal cord.

“A therapeutically effective amount” as used herein refers to the amount of each therapeutic agent of the present disclosure required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons. Empirical considerations, such as the half-life, generally will contribute to the determination of the dosage. For example, therapeutic agents that are compatible with the human immune system, such as polypeptides comprising regions from humanized antibodies or fully human antibodies, may be used to prolong half-life of the polypeptide and to prevent the polypeptide being attacked by the host's immune system.

Frequency of administration may be determined and adjusted over the course of therapy, and is generally, but not necessarily, based on treatment and/or suppression and/or amelioration and/or delay of a disease. Alternatively, sustained continuous release formulations of a polypeptide may be appropriate. Various formulations and devices for achieving sustained release are known in the art. In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays.

The dosing regimen (including the polypeptide used) can vary over time. In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the polypeptide (such as the half-life of the polypeptide, and other considerations well known in the art).

For the purpose of the present disclosure, the appropriate dosage of a therapeutic agent as described herein will depend on the specific agent (or compositions thereof) employed, the formulation and route of administration, the type and severity of the disease, whether the polypeptide is administered for preventive or therapeutic purposes, previous therapy, the subject's clinical history and response to the antagonist, and the discretion of the attending physician. Typically the clinician will administer a polypeptide until a dosage is reached that achieves the desired result.

Administration of one or more polypeptides can be continuous or intermittent, depending, for example, upon the recipient's physiological condition, whether the purpose of the administration is therapeutic or prophylactic, and other factors known to skilled practitioners. The administration of a polypeptide may be essentially continuous over a preselected period of time or may be in a series of spaced dose, e.g., either before, during, or after developing a disease. As used herein, the term “treating” refers to the application or administration of a polypeptide or composition including the polypeptide to a subject in need thereof.

“A subject in need thereof”, refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease. In some embodiments, the subject has CDI. In some embodiments, the subject has cancer. In some embodiments, the subject is a mammal. In some embodiments, the subject is a non-human primate. In some embodiments, the subject is human. Alleviating a disease includes delaying the development or progression of the disease, or reducing disease severity. Alleviating the disease does not necessarily require curative results.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir.

The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

As used herein, a “subject” refers to a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human.

The terms, “patient” and “subject” are used interchangeably herein. A subject can be male or female. A subject can be a fully developed subject (e.g., an adult) or a subject undergoing the developmental process (e.g., a child, infant or fetus). Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with unwanted neuronal activity. In addition, the methods and compositions described herein can be used to treat domesticated animals and/or pets.

It is also envisioned that the modified receptor binding domain of BoNT/A2 described here can be utilized as a delivery tool to target neurons in humans. For example, the modified receptor binding domain of BoNT/A2 can be linked to other therapeutic agents, covalently or non-covalently, and acts as the targeting vehicle to deliver the therapeutic agents to neurons in humans.

Also provided are cells comprising the nucleic acids or nucleic acid vectors, and cells expressing such chimeric molecules. The chimeric molecules in a fusion protein form may be expressed and isolated using the methods disclosed herein.

In some embodiments, such enhanced binding is also specific to a presynaptic nerve terminal. In some embodiments, the presynaptic nerve terminal is in a mammal. In some embodiments, the presynaptic nerve terminal is in a rodent. In some embodiments, the presynaptic nerve terminal is a mouse presynaptic nerve terminal. In some embodiments, the presynaptic nerve terminal is a mouse presynaptic nerve terminal. In some embodiments, the presynaptic nerve terminal is a human presynaptic nerve terminal.

A modified BoNT polypeptide that has enhanced binding affinity to its target cells (e.g., neurons) affords potential for therapeutic use. For example, such modified BoNT polypeptide may be effective at a lower dose. A lower BoNT dose for therapeutic use is generally desirable because less toxin will diffuse to surrounding tissues at the injection site and less neutralizing antibodies may be generated against the BoNT.

### SEQUENCE TABLE

TABLE 1
BONT Sequences

| | | |
|---|---|---|
| 1 | BONT/A2 | MPFVNKQFNYKDPVNGVDIAYIKIPNAGQMQPVKAFKIHNKIWVIPERDTFTNPEE |
| | wildtype | GDLNPPPEAKQVPVSYYDSTYLSTDNEKDNYLKGVTKLFERIYSTDLGRMLLTSIV |
| | | RGIPFWGGSTIDTELKVIDTNCINVIQPDGSYRSEELNLVIIGPSADIIQFECKSFGHD |
| | | VLNLTRNGYGSTQYIRFSPDFTFGFEESLEVDTNPLLGAGKFATDPAVTLAHELIH |
| | | AEHRLYGIAINPNRVFKVNTNAYYEMSGLEVSFEELRTFGGHDAKFIDSLQENEFR |
| | | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSLDEGYNKALN |
| | | DLCIKVNNWDLFFSPSEDNFTNDLDKVEEITADTNIEAAEENISLDLIQQYYLTFDF |
| | | DNEPENISIENLSSDIIGQLEPMPNIERFPNGKKYELDKYTMFHYLRAQEFEHGDSRI |
| | | ILTNSAEEALLKPNVAYTFFSSKYVKKINKAVEAFMFLNWAEELVYDFTDETNEV |
| | | TTMDKIADITIIVPYIGPALNIGNMLSKGEFVEAIIFTGVVAMLEFIPEYALPVFGTF |
| | | AIVSYIANKVLTVQTINNALSKRNEKWDEVYKYTVTNWLAKVNTQIDLIREKMK |
| | | KALENQAEATKAIINYQYNQYTEEEKNNINFNIDDLSSKLNESINSAMININKFLDQ |
| | | CSVSYLMNSMIPYAVKRLKDFDASVRDVLLKYIYDNRGTLVLQVDRLKDEVNNT |
| | | LSADIPFQLSKYVDNKKLLSTFTEYIKNIVNTSILSIVYKKDDLIDLSRYGAKINIGD |
| | | RVYYDSIDKNQIKLINLESSTIEVILKNAIVYNSMYENFSTSFWIKIPKYFSKINLNN |
| | | EYTIINCIENNSGWKVSLNYGEIIWTLQDNKQNIQRVVFKYSQMVNISDYINRWIF |
| | | VTITNNRLTKSKIYINGRLIDQKPISNLGNIHASNKIMFKLDGCRDPRRYIMIKYFNL |
| | | FDKELNEKEIKDLYDSQSNSGILKDFWGNYLQYDKPYYMLNLFDPNKYVDVNNI |
| | | GIRGYMYLKGPRGSVVTTNIYLNSTLYEGTKFIIKKYASGNEDNIVRNNDRVYINV |
| | | VVKNKEYRLATNASQAGVEKILSALEIPDVGNLSQVVVMKSKDDQGIRNKCKMN |
| | | LQDNNGNDIGFIGFHLYDNIAKLVASNWYNRQVGKASRTFGCSWEFIPVDDGWG |
| | | ESSL |
| 2 | BONT/B | MPVTINNFNYNDPIDNNNIIMMEPPFARGTGRYYKAFKITDRIWIIPERYTFGYKPE |
| | wildtype | DFNKSSGIFNRDVCEYYDPDYLNTNDKKNIFLQTMIKLFNRIKSKPLGEKLLEMIIN |
| | (Okra Strain) | GIPYLGDRRVPLEEFNTNIASVTVNKLISNPGEVERKKGIFANLIIFGPGPVLNENET |
| | | IDIGIQNHFASREGFGGIMQMKFCPEYVSVENNVQENKGASIFNRRGYFSDPALIL |
| | | MHELIHVLHGLYGIKVDDLPIVPNEKKFFMQSTDAIQAEELYTFGGQDPSIITPSTD |
| | | KSIYDKVLQNFRGIVDRINKVLVCISDPNININIYKNKFKDKYKFVEDSEGKYSIDV |
| | | ESFDKLYKSLMFGFTETNIAENYKIKTRASYFSDSLPPVKIKNLLDNEIYTIEEGFNI |
| | | SDKDMEKEYRGQNKAINKQAYEEISKEHLAVYKIQMCKSVKAPGICIDVDNEDLF |
| | | FIADKNSFSDDLSKNERIEYNTQSNYIENDFPINELILDTDLISKIELPSENTESLTDF |
| | | NVDVPVYEKQPAIKKIFTDENTIFQYLYSQTFPLDIRDISLTSSFDDALLFSNKVYSF |
| | | FSMDYIKTANKVVEAGLFAGWVKQIVNDFVIEANKSNTMDKIADISLIVPYIGLAL |
| | | NVGNETAKGNFENAFEIAGASILLEFIPELLIPVVGAFLLESYIDNKNKIIKTIDNALT |
| | | KRNEKWSDMYGLIVAQWLSTVNTQFYTIKEGMYKALNYQAQALEEIIKYRYNIY |
| | | SEKEKSNINIDFNDINSKLNEGINQAIDNINNFINGCSVSYLMKKMIPLAVEKLLDF |
| | | DNTLKKNLLNYIDENKLYLIGSAEYEKSKVNKYLKTIMPFDLSIYTNDTILIEMENK |
| | | YNSEILNNIILNLRYKDNNLIDLSGYGAKVEVYDGVELNDKNQFKLTSSANSKIRV |
| | | TQNQNIIFNSVFLDFSVSFWIRIPKYKNDGIQNYIHNEYTIINCMKNNSGWKISIRGN |
| | | RIIWTLIDINGKTKSVFFEYNIREDISEYINRWFFVTITNNLNNAKIYINGKLESNTDI |
| | | KDIREVIANGEIIFKLDGDIDRTQFIWMKYFSIFNTELSQSNIEERYKIQSYSEYLKDF |
| | | WGNPLMYNKEYYMFNAGNKNSYIKLKKDSPVGEILTRSKYNQNSKYINYRDLYI |
| | | GEKFIIRRKSNSQSINDDIVRKEDYIYLDFFNLNQEWRVYTYKYFKKEEEKLFLAPI |
| | | SDSDEFYNTIQIKEYDEQPTYSCQLLFKKDEESTDEIGLIGIHRFYESGIVFEEYKDY |
| | | FCISKWYLKEVKRKPYNLKLGCNWQFIPKDEGWTE |
| 3 | BONT/C | MPITINNFNYSDPVDNKNILYLDTHLNTLANEPEKAFRITGNIWVIPDRESRNSNPN |
| | wildtype | LNKPPRVTSPKSGYYDPNYLSTDSDKDTFLKEIIKLFKRINSREIGEELIYRLSTDIPF |
| | | PGNNNTPINTFDFDVDFNSVDVKTRQGNNWVKTGSINPSVIITGPRENIIDPETSTF |
| | | KLTNNTFAAQEGFGALSIISISPRFMLTYSNATNDVGEGRFSKSEFCMDPILILMHE |
| | | LNHAMHNLYGIAIPNDQTISSVTSNIFYSQYNVKLEYAEIYAFGGPTIDLIPKSARK |
| | | YFEEKALDYYRSIAKRLNSITTANPSSFNKYIGEYKQKLIRKYRFVVESSGEVTVNR |
| | | NKFVELYNELTQIFTEFNYAKIYNVQNRKIYLSNVYTPVTANILDDNVYDIQNGFN |
| | | IPKSNLNVLFMGQNLSRNPALRKVNPENMLYLFTKFCHKAIDGRSLYNKTLDCRE |
| | | LLVKNTDLPFIGDISDVKTDIFLRKDINEETEVIYYPDNVSVDQVILSKNTSEHGQL |
| | | DLLYPSIDSESEILPGENQVFYDNRTQNVDYLNSYYYLESQKLSDNVEDFTFTRSIE |
| | | EALDNSAKVYTYFPTLANKVNAGVQGGLFLMWANDVVEDFTTNILRKDTLDKIS |
| | | DVSAIIPYIGPALNISNSVRRGNFTEAFAVTGVTILLEAFPEFTIPALGAFVIYSKVQE |
| | | RNEIIKTIDNCLEQRIKRWKDSYEWMMGTWLSRIITQFNNISYQMYDSLNYQAGAI |
| | | KAKIDLEYKKYSGSDKENIKSQVENLKNSLDVKISEAMNNINKFIRECSVTYLFKN |
| | | MLPKVIDELNEFDRNTKAKLINLIDSHNIILVGEVDKLKAKVNNSFQNTIPFNIFSYT |
| | | NNSLLKDIINEYFNNINDSKILSLQNRKNTLVDTSGYNAEVSEEGDVQLNPIFPFDF |
| | | KLGSSGEDRGKVIVTQNENIVYNSMYESFSISFWIRINKWVSNLPGYTIIDSVKNNS |
| | | GWSIGIISNFLVFTLKQNEDSEQSINFSYDISNNAPGYNKWFFVTVTNNMMGNMKI |
| | | YINGKLIDTIKVKELTGINFSKTITFEINKIPDTGLITSDSDNINMWIRDFYIFAKELD |
| | | GKDINILFNSLQYTNVVKDYWGNDLRYNKEYYMVNIDYLNRYMYANSRQIVENT |
| | | RRNNNDFNEGYKIIIKRIRGNTNDTRVRGGDILYFDMTINNKAYNLFMKNETMYA |
| | | DNHSTEDIYAIGLREQTKDINDNIIFQIQPMNNTYYYASQIFKSNFNGENISGICSIGT |
| | | YRFRLGGDWYRHNYLVPTVKQGNYASLLESTSTHWGFVPVSE |
| 4 | BONT/D | MTWPVKDFNYSDPVNDNDILYLRIPQNKLITTPVKAFMITQNIWVIPERFSSDTNPS |
| | wildtype | LSKPPRPTSKYQSYYDPSYLSTDEQKDTFLKGIIKLFKRINERDIGKKLINYLVVGSP |
| | | FMGDSSTPEDTFDFTRHTTNIAVEKFENGSWKVTNIITPSVLIFGPLPNILDYTASLT |
| | | LQGQQSNPSFEGFGTLSILKVAPEFLLTFSDVTSNQSSAVLGKSIFCMDPVIALMHE |
| | | LTHSLHQLYGINIPSDKRIRPQVSEGFFSQDGPNVQFEELYTFGGLDVEIIPQIERSQ |
| | | LREKALGHYKDIAKRLNNINKTIPSSWISNIDKYKKIFSEKYNFDKDNTGNFVVNID |
| | | KFNSLYSDLTNVMSEVVYSSQYNVKNRTHYFSRHYLPVFANILDDNIYTIRDGFNL |
| | | TNKGFNIENSGQNIERNPALQKLSSESVVDLFTKVCLRLTKNSRDDSTCIKVKNNR |
| | | LPYVADKDSISQEIFENKIITDETNVQNYSDKFSLDESILDGQVPINPEIVDPLLPNV |
| | | NMEPLNLPGEEIVFYDDITKYVDYLNSYYYLESQKLSNNVENITLTTSVEEALGYS |
| | | NKIYTFLPSLAEKVNKGVQAGLFLNWANEVVEDFTTNIMKKDTLDKISDVSVIIPY |
| | | IGPALNIGNSALRGNFNQAFATAGVAFLLEGFPEFTIPALGVFTFYSSIQEREKIIKTI |
| | | ENCLEQRVKRWKDSYQWMVSNWLSRITTQFNHINYQMYDSLSYQADAIKAKIDL |
| | | EYKKYSGSDKENIKSQVENLKNSLDVKISEAMNNINKFIRECSVTYLFKNMLPKVI |
| | | DELNKFDLRTKTELINLIDSHNIILVGEVDRLKAKVNESFENTMPFNIFSYTNNSLL |
| | | KDIINEYFNSINDSKILSLQNKKNALVDTSGYNAEVRVGDNVQLNTIYTNDFKLSS |
| | | SGDKIIVNLNNNILYSAIYENSSVSFWIKISKDLTNSHNEYTIINSIEQNSGWKLCIRN |
| | | GNIEWILQDVNRKYKSLIFDYSESLSHTGYTNKWFFVTITNNIMGYMKLYINGELK |
| | | QSQKIEDLDEVKLDKTIVFGIDENIDENQMLWIRDFNIFSKELSNEDINIVYEGQILR |
| | | NVIKDYWGNPLKFDTEYYIINDNYIDRYIAPESNVLVLVQYPDRSKLYTGNPITIKS |
| | | VSDKNPYSRILNGDNIILHMLYNSRKYMIIRDTDTIYATQGGECSQNCVYALKLQS |
| | | NLGNYGIGIFSIKNIVSKNKYCSQIFSSFRENTMLLADIYKPWRFSFKNAYTPVAVT |
| | | NYETKLLSTSSFWKFISRDPGWVE |
| 5 | BONT/E | MPKINSFNYNDPVNDRTILYIKPGGCQEFYKSFNIMKNIWIIPERNVIGTTPQDFHPP |
| | wildtype | TSLKNGDSSYYDPNYLQSDEEKDRFLKIVTKIFNRINNNLSGGILLEELSKANPYLG |
| | | NDNTPDNQFHIGDASAVEIKFSNGSQDILLPNVIIMGAEPDLFETNSSNISLRNNYM |
| | | PSNHRFGSIAIVTFSPEYSFRFNDNCMNEFIQDPALTLMHELIHSLHGLYGAKGITT |
| | | KYTITQKQNPLITNIRGTNIEEFLTFGGTDLNIITSAQSNDIYTNLLADYKKIASKLS |
| | | KVQVSNPLLNPYKDVFEAKYGLDKDASGIYSVNINKENDIFKKLYSFTEFDLRTKF |
| | | QVKCRQTYIGQYKYFKLSNLLNDSIYNISEGYNINNLKVNFRGQNANLNPRIITPIT |
| | | GRGLVKKIIRFCKNIVSVKGIRKSICIEINNGELFFVASENSYNDDNINTPKEIDDTV |
| | | TSNNNYENDLDQVILNFNSESAPGLSDEKLNLTIQNDAYIPKYDSNGTSDIEQHDV |
| | | NELNVFFYLDAQKVPEGENNVNLTSSIDTALLEQPKIYTFFSSEFINNVNKPVQAAL |
| | | FVSWIQQVLVDFTTEANQKSTVDKIADISIVVPYIGLALNIGNEAQKGNFKDALEL |
| | | LGAGILLEFEPELLIPTILVFTIKSFLGSSDNKNKVIKAINNALKERDEKWKEVYSFI |
| | | VSNWMTKINTQFNKRKEQMYQALQNQVNAIKTIIESKYNSYTLEEKNELTNKYDI |
| | | KQIENELNQKVSIAMNNIDRFLTESSISYLMKIINEVKINKLREYDENVKTYLLNYII |
| | | QHGSILGESQQELNSMVTDTLNNSIPFKLSSYTDDKILISYFNKFFKRIKSSSVLNMR |
| | | YKNDKYVDTSGYDSNININGDVYKYPTNKNQFGIYNDKLSEVNISQNDYIIYDNK |
| | | YKNFSISFWVRIPNYDNKIVNVNNEYTIINCMRDNNSGWKVSLNHNEIIWTFEDNR |
| | | GINQKLAFNYGNANGISDYINKWIFVTITNDRLGDSKLYINGNLIDQKSILNLGNIH |
| | | VSDNILFKIVNCSYTRYIGIRYFNIFDKELDETEIQTLYSNEPNTNILKDFWGNYLLY |
| | | DKEYYLLNVLKPNNFIDRRKDSTLSINNIRSTILLANRLYSGIKVKIQRVNNSSTND |
| | | NLVRKNDQVYINFVASKTHLFPLYADTATTNKEKTIKISSSGNRFNQVVVMNSVG |
| | | NCTMNFKNNNGNNIGLLGFKADTVVASTWYYTHMRDHTNSNGCFWNFISEEHG |
| | | WQEK |
| 6 | BONT/F | MPVVINSFNYNDPVNDDTILYMQIPYEEKSKKYYKAFEIMRNVWIIPERNTIGTDP |
| | wildtype | SDFDPPASLENGSSAYYDPNYLTTDAEKDRYLKTTIKLFKRINSNPAGEVLLQEISY |
| | | AKPYLGNEHTPINEFHPVTRTTSVNIKSSTNVKSSIILNLLVLGAGPDIFENSSYPVR |
| | | KLMDSGGVYDPSNDGFGSINIVTFSPEYEYTFNDISGGYNSSTESFIADPAISLAHEL |
| | | IHALHGLYGARGVTYKETIKVKQAPLMIAEKPIRLEEFLTFGGQDLNIITSAMKEKI |
| | | YNNLLANYEKIATRLSRVNSAPPEYDINEYKDYFQWKYGLDKNADGSYTVNENK |
| | | FNEIYKKLYSFTEIDLANKFKVKCRNTYFIKYGFLKVPNLLDDDIYTVSEGFNIGNL |
| | | AVNNRGQNIKLNPKIIDSIPDKGLVEKIVKFCKSVIPRKGTKAPPRLCIRVNNRELFF |
| | | VASESSYNENDINTPKEIDDTTNLNNNYRNNLDEVILDYNSETIPQISNQTLNTLVQ |
| | | DDSYVPRYDSNGTSEIEEHNVVDLNVFFYLHAQKVPEGETNISLTSSIDTALSEESQ |
| | | VYTFFSSEFINTINKPVHAALFISWINQVIRDFTTEATQKSTFDKIADISLVVPYVGL |
| | | ALNIGNEVQKENFKEAFELLGAGILLEFVPELLIPTILVFTIKSFIGSSENKNKIIKAIN |
| | | NSLMERETKWKEIYSWIVSNWLTRINTQFNKRKEQMYQALQNQVDAIKTVIEYK |
| | | YNNYTSDERNRLESEYNINNIREELNKKVSLAMENIERFITESSIFYLMKLINEAKV |
| | | SKLREYDEGVKEYLLDYISEHRSILGNSVQELNDLVTSTLNNSIPFELSSYTNDKILI |
| | | LYFNKLYKKIKDNSILDMRYENNKFIDISGYGSNISINGDVYIYSTNRNQFGIYSSKP |
| | | SEVNIAQNNDIIYNGRYQNFSISFWVRIPKYFNKVNLNNEYTIIDCIRNNNSGWKIS |
| | | LNYNKIIWTLQDTAGNNQKLVFNYTQMISISDYINKWIFVTITNNRLGN |
| | | SRIYINGNLIDEKSISNLGDIHVSDNILFKIVGCNDTRYVGIRYFKVFDTELGKTEIET |
| | | LYSDEPDPSILKDFWGNYLLYNKRYYLLNLLRTDKSITQNSNFLNINQQRGVYQK |
| | | PNIFSNTRLYTGVEVIIRKNGSTDISNTDNFVRKNDLAYINVVDRDVEYRLYADISI |
| | | AKPEKIIKLIRTSNSNNSLGQIIVMDSIGNNCTMNFQNNNGGNIGLLGFHSNNLVAS |
| | | SWYYNNIRKNTSSNGCFWSFISKEHGWQEN |
| 7 | BONT/G | MPVNIKXFNYNDPINNDDIIMMEPFNDPGPGTYYKAFRIIDRIWIVPERFTYGFQPD |
| | wildtype | QFNASTGVFSKDVYEYYDPTYLKTDAEKDKFLKTMIKLFNRINSKPSGQRLLDMI |
| | | VDAIPYLGNASTPPDKFAANVANVSINKKIIQPGAEDQIKGLMTNLIIFGPGPVLSD |
| | | NFTDSMIMNGHSPISEGFGARMMIRFCPSCLNVFNNVQENKDTSIFSRRAYFADPA |
| | | LTLMHELIHVLHGLYGIKISNLPITPNTKEFFMQHSDPVQAEELYTFGGHDPSVISPS |
| | | TDMNIYNKALQNFQDIANRLNIVSSAQGSGIDISLYKQIYKNKYDFVEDPNGKYSV |
| | | DKDKFDKLYKALMFGFTETNLAGEYGIKTRYSYFSEYLPPIKTEKLLDNTIYTQNE |
| | | GFNIASKNLKTEFNGQNKAVNKEAYEEISLEHLVIYRIAMCKPVMYKNTGKSEQCI |
| | | IVNNEDLFFIANKDSFSKDLAKAETIAYNTQNNTIENNFSIDQLILDNDLSSGIDLPN |
| | | ENTEPFTNFDDIDIPVYIKQSALKKIFVDGDSLFEYLHAQTFPSNIENLQLTNSLNDA |
| | | LRNNNKVYTFFSTNLVEKANTVVGASLFVNWVKGVIDDFTSESTQKSTIDKVSDV |
| | | SIIIPYIGPALNVGNETAKENFKNAFEIGGAAILMEFIPELIVPIVGFFTLESYVGNKG |
| | | HIIMTISNALKKRDQKWTDMYGLIVSQWLSTVNTQFYTIKERMYNALNNQSQAIE |
| | | KIIEDQYNRYSEEDKMNINIDENDIDFKLNQSINLAINNIDDFINQCSISYLMNRMIP |
| | | LAVKKLKDFDDNLKRDLLEYIDTNELYLLDEVNILKSKVNRHLKDSIPFDLSLYTK |
| | | DTILIQVENNYISNISSNAILSLSYRGGRLIDSSGYGATMNVGSDVIFNDIGNGQFKL |
| | | NNSENSNITAHQSKFVVYDSMFDNESINFWVRTPKYNNNDIQTYLQNEYTIISCIK |
| | | NDSGWKVSIKGNRIIWTLIDVNAKSKSIFFEYSIKDNISDYINKWFSITITNDRLGNA |
| | | NIYINGSLKKSEKILNLDRINSSNDIDFKLINCTDTTKFVWIKDFNIFGRELNATEVS |
| | | SLYWIQSSTNTLKDFWGNPLRYDTQYYLFNQGMQNIYIKYFSKASMGETAPRTNF |
| | | NNAAINYQNLYLGLRFIIKKASNSRNINNDNIVREGDYIYLNIDNISDESYRVYVLV |
| | | NSKEIQTQLFLAPINDDPTFYDVLQIKKYYEKTTYNCQILCEKDTKTFGLFGIGKFV |
| | | KDYGYVWDTYDNYFCISQWYLRRISENINKLRLGCNWQFIPVDEGWTE |
| 8 | BONT/H | MPVVINSFNYDDPVNDNTIIYIRPPYYETSNTYFKAFQIMDNVWIIPERYRLGIDPSL |
| | wildtype | FNPPVSLKAGSDGYFDPNYLSTNTEKNKYLQIMIKLFKRINSKPAGQILLEEIKNAIP |
| | | YLGNSYTQEEQFTTNNRTVSFNVKLANGNIVQQMANLIIWGPGPDLTTNKTGGIIY |
| | | SPYQSMEATPYKDGFGSIMTVEFSPEYATAFNDISIASHSPSLFIKDPALILMHELIH |
| | | VLHGLYGTYITEYKITPNVVQSYMKVTKPITSAEFLTFGGRDRNIVPQSIQSQLYNK |
| | | VLSDYKRIASRLNKVNTATALINIDEFKNLYEWKYQFAKDSNGVYSVDLNKFEQL |
| | | YKKIYSFTEFNLAYEFKIKTRLGYLAENFGPFYLPNLLDDSIYTEVDGFNIGALSIN |
| | | YQGQNIGSDINSIKKLQGQGVVSRVVRLCSNSNTKNSLCITVNNRDLFFIASQESY |
| | | GENTINTYKEIDDTTTLDPSFEDILDKVILNFNEQVIPQMPNRNVSTDIQKDNYIPK |
| | | YDYNRTDIIDSYEVGRNYNTFFYLNAQKESPNESNITLTSSFDTGLLEGSKVYTFFS |
| | | SDFINNINKPVQALLFIEWVKQVIRDFTTEATKTSTVDKLKDISLVVPYIGLALNIG |
| | | DEIYKQHFAEAVELVGAGLLLEFSPEFLIPTLLIFTIKGYLTGSIRDKDKIIKTLDNAL |
| | | NVRDQKWKELYRWVVSKWLTTINTQFNKRKEQMYKALKNQATAIKKIIENKYN |
| | | NYTTDEKSKIDSSYNINEIERTLNEKINLAMKNIEQFITESSIAYLINIINNETIQKLKS |
| | | YDDLVRRYLLGYIRNHSSILGNSVEELNSKVNNHLDNGIPFELSSYTNDSLLIRYFN |
| | | KNYGELKYNCILNIKYEMDRDKLVDSSGYRSRINIGTGVKFSEIDKNQVQLSNLES |
| | | SKIEVILNNGVIYNSMYENFSTSFWIRIPKYFRNINNEYKIISCMQNNSGWEVSLNFS |
| | | NMNSKIIWTLQDTEGIKKTVVFQYTQNINISDYINRWIFVTITNNRLSNSKIYINGRL |
| | | INEESISDLGNIHASNNIMFKLDGCRDPHRYIWIKYFNLFDKELNKKEIKDLYDNQS |
| | | NSGILKDFWGDYLQYDKPYYMLNLYDPNKYLDVNNVGIRGYMYLKGPRGRIVT |
| | | TNIYLNSTLYMGTKFIIKKYASGNKDNIVRNNDRVYINVVVKNKEYRLATNASQA |
| | | GVEKILSAVEIPDVGNLSQVVVMKSENDQGIRNKCKMNLQDNNGNDIGFIGFHQF |
| | | NNIAKLVASNWYNRQIGKASRTFGCSWEFIPVDDGWGESSL |
| 9 | BONT/X | MKLEINKFNYNDPIDGINVITMRPPRHSDKINKGKGPFKAFQVIKNIWIVPERYNFT |
| | wildtype | NNTNDLNIPSEPIMEADAIYNPNYLNTPSEKDEFLQGVIKVLERIKSKPEGEKLLELI |
| | | SSSIPLPLVSNGALTLSDNETIAYQENNNIVSNLQANLVIYGPGPDIANNATYGLYS |
| | | TPISNGEGTLSEVSFSPFYLKPFDESYGNYRSLVNIVNKFVKREFAPDPASTLMHEL |
| | | VHVTHNLYGISNRNFYYNFDTGKIETSRQQNSLIFEELLTFGGIDSKAISSLIIKKIIE |
| | | TAKNNYTTLISERLNTVTVENDLLKYIKNKIPVQGRLGNFKLDTAEFEKKLNTILF |
| | | VLNESNLAQRFSILVRKHYLKERPIDPIYVNILDDNSYSTLEGFNISSQGSNDFQGQ |
| | | LLESSYFEKIESNALRAFIKICPRNGLLYNAIYRNSKNYLNNIDLEDKKTTSKTNVS |
| | | YPCSLLNGCIEVENKDLFLISNKDSLNDINLSEEKIKPETTVFFKDKLPPQDITLSNY |
| | | DFTEANSIPSISQQNILERNEELYEPIRNSLFEIKTIYVDKLTTFHFLEAQNIDESIDSS |
| | | KIRVELTDSVDEALSNPNKVYSPFKNMSNTINSIETGITSTYIFYQWLRSIVKDFSDE |
| | | TGKIDVIDKSSDTLAIVPYIGPLLNIGNDIRHGDFVGAIELAGITALLEYVPEFTIPIL |
| | | VGLEVIGGELAREQVEAIVNNALDKRDQKWAEVYNITKAQWWGTIHLQINTRLA |
| | | HTYKALSRQANAIKMNMEFQLANYKGNIDDKAKIKNAISETEILLNKSVEQAMKN |
| | | TEKFMIKLSNSYLTKEMIPKVQDNLKNFDLETKKTLDKFIKEKEDILGTNLSSSLRR |
| | | KVSIRLNKNIAFDINDIPFSEFDDLINQYKNEIEDYEVLNLGAEDGKIKDLSGTTSDI |
| | | NIGSDIELADGRENKAIKIKGSENSTIKIAMNKYLRFSATDNFSISFWIKHPKPTNLL |
| | | NNGIEYTLVENFNQRGWKISIQDSKLIWYLRDHNNSIKIVTPDYIAFNGWNLITITN |
| | | NRSKGSIVYVNGSKIEEKDISSIWNTEVDDPIIFRLKNNRDTQAFTLLDQFSIYRKEL |
| | | NQNEVVKLYNYYFNSNYIRDIWGNPLQYNKKYYLQTQDKPGKGLIREYWSSFGY |
| | | DYVILSDSKTITFPNNIRYGALYNGSKVLIKNSKKLDGLVRNKDFIQLEIDGYNMGI |
| | | SADRFNEDTNYIGTTYGTTHDLTTDFEIIQRQEKYRNYCQLKTPYNIFHKSGLMST |
| | | ETSKPTFHDYRDWVYSSAWYFQNYENLNLRKHTKTNWYFIPKDEGWDED |
| 10 | BONT/EN | MVTINDLHYSDPIDEDNIINMRIPLYDLEVDDQFINHNVPDLKAFQVFPNVWVVPE |
| | wildtype | RYTFYSTMKNLDAPANPSRSSYYDPTYLQSDAEKEVFLQQMILLFKRINSTQEGQ |
| | | QFLNLLSRSIPVPYESNGDVAMGTTQVIKQMDDKGNVLKHRRAHIIIYGPGPDLM |
| | | AKGSKALTKSRETGRGCMAEIYFSPMYHKTYSTKLINKNSLVDKSVQEFVPDPAV |
| | | TLIHELCHGLHALYGIDLGNVGSWEFNSNPNSLFSSWFSSKEAVNFEEVMTFGGE |
| | | DVKVIKSEIDKKIPGILNLIKTTVEPIINKITDPHDEMLQCLQSKYPSLKGTLGQFFF |
| | | DDTQLEKDIRDLWMVMNETMFAENLKALTRARYLVPKVENIVQVDILSPNVYTI |
| | | DKGFNHLSKGFKGQSVSQSYFRKISALARGAVVRACPNPHFSSQRGLSSCIEILED |
| | | DLFIMSSKDSFTDTDFSEPSVGPVSYKAKKGADTILDSTLSNYDFSKEINFTSTVPII |
| | | TVEDPLETDEDVPVISEDRTVYVDDYTTFHFLEAQKIGKEVVPTQTKVVFTTNME |
| | | EALFDSKKVYTVFENTASRINEAGTGIANGMMFYQWLKGIVQDFTEEATQKDTFD |
| | | KISDVTMIVPYLGNILNIGNDIRKGDFMGAVELGGVTILLEAIPELTLPVLIGLTIIED |
| | | ELEKEQVSQTVYNVLDKRDEKWEEVYGFVKQQWWWMVHTQFETRILHAYQAL |
| | | NHQVEAIKANMTYQLANYRGNQEDKELLEKAIDDTLQSLYYAVDQAMHNIKRFL |
| | | IQSSKSYLLNQMLPKTKEQLLAFDQQTLRNVNDFINKNQGVLGESLAKDLKKKVE |
| | | KRLTSLPVFNLEDLPISEFEDLIHSHEIDIQDSEVLNIGVNNGKIQDLSGENTPLTLGE |
| | | NLHIVNGRDNQAVRLNNQLDSKLEIQSRPNIHFTAFEDFSISIWIRCSMLRNNRNRG |
| | | QKYTIIQQFNKYGWQLAIQDSVFVWTLHDTFNNQIQLTSGSALTNKNYLLQNFWL |
| | | HITVTNKRSEKSRLYINGVLQDQKDISVLGNCHPKEPILFSIQDNSDPNYFVRFEQF |
| | | NVYRKALTDSEVNRLYWKYFEGSYLRDVWGERLTYNRDYYMQLSTLPGRGIKR |
| | | EYRTWSGFDYIILSELGTQKIPTHEVTYPKLYQGQKITIHSDGKNLEPHVKSNKNIR |
| | | LKIDDFYIGVVNPFKLPEWRPESGAYVVTTYNHAEDLCLYFRTRSSSQSLYYGQLI |
| | | MNDGRNKSLLNYTLKGSTYWIWSSAWYYENYNTSSKTAGNWYFIPVDEGWKED |
| 11 | BONT/A1 | MPFVNKQFNYKDPVNGVDIAYIKIPNAGQMQPVKAFKIHNKIWVIPERDTFTNPEE |
| | wildtype | GDLNPPPEAKQVPVSYYDSTYLSTDNEKDNYLKGVTKLFERIYSTDLGRMLLTSIV |
| | | RGIPFWGGSTIDTELKVIDTNCINVIQPDGSYRSEELNLVIIGPSADIIQFECKSFGHE |
| | | VLNLTRNGYGSTQYIRFSPDFTFGFEESLEVDTNPLLGAGKFATDPAVTLAHELIH |
| | | AGHRLYGIAINPNRVFKVNTNAYYEMSGLEVSFEELRTFGGHDAKFIDSLQENEFR |
| | | LYYYNKFKDIASTLNKAKSIVGTTASLQYMKNVFKEKYLLSEDTSGKFSVDKLKF |
| | | DKLYKMLTEIYTEDNFVKFFKVLNRKTYLNFDKAVFKINIVPKVNYTIYDGFNLR |
| | | NTNLAANFNGQNTEINNMNFTKLKNFTGLFEFYKLLCVRGIITSKTKSLDKGYNK |
| | | ALNDLCIKVNNWDLFFSPSEDNFTNDLNKGEEITSDTNIEAAEENISLDLIQQYYLT |
| | | FNFDNEPENISIENLSSDIIGQLELMPNIERFPNGKKYELDKYTMFHYLRAQEFEHG |
| | | KSRIALTNSVNEALLNPSRVYTFFSSDYVKKVNKATEAAMFLGWVEQLVYDFTD |
| | | ETSEVSTTDKIADITIIIPYIGPALNIGNMLYKDDFVGALIFSGAVILLEFIPEIAIPVLG |
| | | TFALVSYIANKVLTVQTIDNALSKRNEKWDEVYKYIVTNWLAKVNTQIDLIRKKM |
| | | KEALENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKVNN |
| | | TLSTDIPFQLSKYVDNQRLLSTFTEYIKNIINTSILNLRYESNHLIDLSRYASKINIGS |
| | | KVNFDPIDKNQIQLFNLESSKIEVILKNAIVYNSMYENFSTSFWIRIPKYFNSISLNN |
| | | EYTIINCMENNSGWKVSLNYGEIIWTLQDTQEIKQRVVFKYSQMINISDYINRWIF |
| | | VTITNNRLNNSKIYINGRLIDQKPISNLGNIHASNNIMFKLDGCRDTHRYIWIKYFN |
| | | LFDKELNEKEIKDLYDNQSNSGILKDFWGDYLQYDKPYYMLNLYDPNKYVDVN |
| | | NVGIRGYMYLKGPRGSVMTTNIYLNSSLYRGTKFIIKKYASGNKDNIVRNNDRVY |
| | | INVVVKNKEYRLATNASQAGVEKILSALEIPDVGNLSQVVVMKSKNDQGITNKCK |
| | | MNLQDNNGNDIGFIGFHQFNNIAKLVASNWYNRQIERSSRTLGCSWEFIPVDDGW |
| | | GERPL |
| 12 | BONT/A2- | MPFVNKQFNYKDPVNGVDIAYIKIPNAGQMQPVKAFKIHNKIWVIPERDTFTNPEE |
| | R1064W | GDLNPPPEAKQVPVSYYDSTYLSTDNEKDNYLKGVTKLFERIYSTDLGRMLLTSIV |
| | | RGIPFWGGSTIDTELKVIDTNCINVIQPDGSYRSEELNLVIIGPSADIIQFECKSFGHD |
| | | VLNLTRNGYGSTQYIRFSPDFTFGFEESLEVDTNPLLGAGKFATDPAVTLAHELIH |
| | | AEHRLYGIAINPNRVFKVNTNAYYEMSGLEVSFEELRTFGGHDAKFIDSLQENEFR |
| | | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSLDEGYNKALN |
| | | DLCIKVNNWDLFFSPSEDNFTNDLDKVEEITADTNIEAAEENISLDLIQQYYLTFDF |
| | | DNEPENISIENLSSDIIGQLEPMPNIERFPNGKKYELDKYTMFHYLRAQEFEHGDSRI |
| | | ILTNSAEEALLKPNVAYTFFSSKYVKKINKAVEAFMFLNWAEELVYDFTDETNEV |
| | | TTMDKIADITIIVPYIGPALNIGNMLSKGEFVEAIIFTGVVAMLEFIPEYALPVFGTF |
| | | AIVSYIANKVLTVQTINNALSKRNEKWDEVYKYTVTNWLAKVNTQIDLIREKMK |
| | | KALENQAEATKAIINYQYNQYTEEEKNNINFNIDDLSSKLNESINSAMININKFLDQ |
| | | CSVSYLMNSMIPYAVKRLKDFDASVRDVLLKYIYDNRGTLVLQVDRLKDEVNNT |
| | | LSADIPFQLSKYVDNKKLLSTFTEYIKNIVNTSILSIVYKKDDLIDLSRYGAKINIGD |
| | | RVYYDSIDKNQIKLINLESSTIEVILKNAIVYNSMYENFSTSFWIKIPKYFSKINLNN |
| | | EYTIINCIENNSGWKVSLNYGEIIWTLQDNKQNIQRVVFKYSQMVNISDYINRWIF |
| | | VTITNNRLTKSKIYINGRLIDQKPISNLGNIHASNKIMFKLDGCRDPWRYIMIKYFN |
| | | LFDKELNEKEIKDLYDSQSNSGILKDFWGNYLQYDKPYYMLNLFDPNKYVDVNN |
| | | IGIRGYMYLKGPRGSVVTTNIYLNSTLYEGTKFIIKKYASGNEDNIVRNNDRVYIN |
| | | VVVKNKEYRLATNASQAGVEKILSALEIPDVGNLSQVVVMKSKDDQGIRNKCKM |
| | | NLQDNNGNDIGFIGFHLYDNIAKLVASNWYNRQVGKASRTFGCSWEFIPVDDGW |
| | | GESSL |
| 13 | BONT/A2- | MPFVNKQFNYKDPVNGVDIAYIKIPNAGQMQPVKAFKIHNKIWVIPERDTFTNPEE |
| | R1064F | GDLNPPPEAKQVPVSYYDSTYLSTDNEKDNYLKGVTKLFERIYSTDLGRMLLTSIV |
| | | RGIPFWGGSTIDTELKVIDTNCINVIQPDGSYRSEELNLVIIGPSADIIQFECKSFGHD |
| | | VLNLTRNGYGSTQYIRFSPDFTFGFEESLEVDTNPLLGAGKFATDPAVTLAHELIH |
| | | AEHRLYGIAINPNRVFKVNTNAYYEMSGLEVSFEELRTFGGHDAKFIDSLQENEFR |
| | | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSLDEGYNKALN |
| | | DLCIKVNNWDLFFSPSEDNFTNDLDKVEEITADTNIEAAEENISLDLIQQYYLTFDF |
| | | DNEPENISIENLSSDIIGQLEPMPNIERFPNGKKYELDKYTMFHYLRAQEFEHGDSRI |
| | | ILTNSAEEALLKPNVAYTFFSSKYVKKINKAVEAFMFLNWAEELVYDFTDETNEV |
| | | TTMDKIADITIIVPYIGPALNIGNMLSKGEFVEAIIFTGVVAMLEFIPEYALPVFGTF |
| | | AIVSYIANKVLTVQTINNALSKRNEKWDEVYKYTVTNWLAKVNTQIDLIREKMK |
| | | KALENQAEATKAIINYQYNQYTEEEKNNINFNIDDLSSKLNESINSAMININKFLDQ |
| | | CSVSYLMNSMIPYAVKRLKDFDASVRDVLLKYIYDNRGTLVLQVDRLKDEVNNT |
| | | LSADIPFQLSKYVDNKKLLSTFTEYIKNIVNTSILSIVYKKDDLIDLSRYGAKINIGD |
| | | RVYYDSIDKNQIKLINLESSTIEVILKNAIVYNSMYENFSTSFWIKIPKYFSKINLNN |
| | | EYTIINCIENNSGWKVSLNYGEIIWTLQDNKQNIQRVVFKYSQMVNISDYINRWIF |
| | | VTITNNRLTKSKIYINGRLIDQKPISNLGNIHASNKIMFKLDGCRDPFRYIMIKYFNL |
| | | FDKELNEKEIKDLYDSQSNSGILKDFWGNYLQYDKPYYMLNLFDPNKYVDVNNI |
| | | GIRGYMYLKGPRGSVVTTNIYLNSTLYEGTKFIIKKYASGNEDNIVRNNDRVYINV |
| | | VVKNKEYRLATNASQAGVEKILSALEIPDVGNLSQVVVMKSKDDQGIRNKCKMN |
| | | LQDNNGNDIGFIGFHLYDNIAKLVASNWYNRQVGKASRTFGCSWEFIPVDDGWG |
| | | ESSL |
| 14 | BONT/A2- | MPFVNKQFNYKDPVNGVDIAYIKIPNAGQMQPVKAFKIHNKIWVIPERDTFTNPEE |
| | R1064H | GDLNPPPEAKQVPVSYYDSTYLSTDNEKDNYLKGVTKLFERIYSTDLGRMLLTSIV |
| | | RGIPFWGGSTIDTELKVIDTNCINVIQPDGSYRSEELNLVIIGPSADIIQFECKSFGHD |
| | | VLNLTRNGYGSTQYIRFSPDFTFGFEESLEVDTNPLLGAGKFATDPAVTLAHELIH |
| | | AEHRLYGIAINPNRVFKVNTNAYYEMSGLEVSFEELRTFGGHDAKFIDSLQENEFR |
| | | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSLDEGYNKALN |
| | | DLCIKVNNWDLFFSPSEDNFTNDLDKVEEITADTNIEAAEENISLDLIQQYYLTFDF |
| | | DNEPENISIENLSSDIIGQLEPMPNIERFPNGKKYELDKYTMFHYLRAQEFEHGDSRI |
| | | ILTNSAEEALLKPNVAYTFFSSKYVKKINKAVEAFMFLNWAEELVYDFTDETNEV |
| | | TTMDKIADITIIVPYIGPALNIGNMLSKGEFVEAIIFTGVVAMLEFIPEYALPVFGTF |
| | | AIVSYIANKVLTVQTINNALSKRNEKWDEVYKYTVTNWLAKVNTQIDLIREKMK |
| | | KALENQAEATKAIINYQYNQYTEEEKNNINFNIDDLSSKLNESINSAMININKFLDQ |
| | | CSVSYLMNSMIPYAVKRLKDFDASVRDVLLKYIYDNRGTLVLQVDRLKDEVNNT |
| | | LSADIPFQLSKYVDNKKLLSTFTEYIKNIVNTSILSIVYKKDDLIDLSRYGAKINIGD |
| | | RVYYDSIDKNQIKLINLESSTIEVILKNAIVYNSMYENFSTSFWIKIPKYFSKINLNN |
| | | EYTIINCIENNSGWKVSLNYGEIIWTLQDNKQNIQRVVFKYSQMVNISDYINRWIF |
| | | VTITNNRLTKSKIYINGRLIDQKPISNLGNIHASNKIMFKLDGCRDPHRYIMIKYFNL |
| | | FDKELNEKEIKDLYDSQSNSGILKDFWGNYLQYDKPYYMLNLFDPNKYVDVNNI |
| | | GIRGYMYLKGPRGSVVTTNIYLNSTLYEGTKFIIKKYASGNEDNIVRNNDRVYINV |
| | | VVKNKEYRLATNASQAGVEKILSALEIPDVGNLSQVVVMKSKDDQGIRNKCKMN |
| | | LQDNNGNDIGFIGFHLYDNIAKLVASNWYNRQVGKASRTFGCSWEFIPVDDGWG |
| | | ESSL |
| 15 | BONT/A2- | MPFVNKQFNYKDPVNGVDIAYIKIPNAGQMQPVKAFKIHNKIWVIPERDTFTNPEE |
| | R1064L | GDLNPPPEAKQVPVSYYDSTYLSTDNEKDNYLKGVTKLFERIYSTDLGRMLLTSIV |
| | | RGIPFWGGSTIDTELKVIDTNCINVIQPDGSYRSEELNLVIIGPSADIIQFECKSFGHD |
| | | VLNLTRNGYGSTQYIRFSPDFTFGFEESLEVDTNPLLGAGKFATDPAVTLAHELIH |
| | | AEHRLYGIAINPNRVFKVNTNAYYEMSGLEVSFEELRTFGGHDAKFIDSLQENEFR |
| | | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSLDEGYNKALN |
| | | DLCIKVNNWDLFFSPSEDNFTNDLDKVEEITADTNIEAAEENISLDLIQQYYLTFDF |
| | | DNEPENISIENLSSDIIGQLEPMPNIERFPNGKKYELDKYTMFHYLRAQEFEHGDSRI |
| | | ILTNSAEEALLKPNVAYTFFSSKYVKKINKAVEAFMFLNWAEELVYDFTDETNEV |
| | | TTMDKIADITIIVPYIGPALNIGNMLSKGEFVEAIIFTGVVAMLEFIPEYALPVFGTF |
| | | AIVSYIANKVLTVQTINNALSKRNEKWDEVYKYTVTNWLAKVNTQIDLIREKMK |
| | | KALENQAEATKAIINYQYNQYTEEEKNNINFNIDDLSSKLNESINSAMININKFLDQ |
| | | CSVSYLMNSMIPYAVKRLKDFDASVRDVLLKYIYDNRGTLVLQVDRLKDEVNNT |
| | | LSADIPFQLSKYVDNKKLLSTFTEYIKNIVNTSILSIVYKKDDLIDLSRYGAKINIGD |
| | | RVYYDSIDKNQIKLINLESSTIEVILKNAIVYNSMYENFSTSFWIKIPKYFSKINLNN |
| | | EYTIINCIENNSGWKVSLNYGEIIWTLQDNKQNIQRVVFKYSQMVNISDYINRWIF |
| | | VTITNNRLTKSKIYINGRLIDQKPISNLGNIHASNKIMFKLDGCRDPLRYIMIKYFNL |
| | | FDKELNEKEIKDLYDSQSNSGILKDFWGNYLQYDKPYYMLNLFDPNKYVDVNNI |
| | | GIRGYMYLKGPRGSVVTTNIYLNSTLYEGTKFIIKKYASGNEDNIVRNNDRVYINV |
| | | VVKNKEYRLATNASQAGVEKILSALEIPDVGNLSQVVVMKSKDDQGIRNKCKMN |
| | | LQDNNGNDIGFIGFHLYDNIAKLVASNWYNRQVGKASRTFGCSWEFIPVDDGWG |
| | | ESSL |
| 16 | BONT/A2- | MPFVNKQFNYKDPVNGVDIAYIKIPNAGQMQPVKAFKIHNKIWVIPERDTFTNPEE |
| | S1294R | GDLNPPPEAKQVPVSYYDSTYLSTDNEKDNYLKGVTKLFERIYSTDLGRMLLTSIV |
| | | RGIPFWGGSTIDTELKVIDTNCINVIQPDGSYRSEELNLVIIGPSADIIQFECKSFGHD |
| | | VLNLTRNGYGSTQYIRFSPDFTFGFEESLEVDTNPLLGAGKFATDPAVTLAHELIH |
| | | AEHRLYGIAINPNRVFKVNTNAYYEMSGLEVSFEELRTFGGHDAKFIDSLQENEFR |
| | | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSLDEGYNKALN |
| | | DLCIKVNNWDLFFSPSEDNFTNDLDKVEEITADTNIEAAEENISLDLIQQYYLTFDF |
| | | DNEPENISIENLSSDIIGQLEPMPNIERFPNGKKYELDKYTMFHYLRAQEFEHGDSRI |
| | | ILTNSAEEALLKPNVAYTFFSSKYVKKINKAVEAFMFLNWAEELVYDFTDETNEV |
| | | TTMDKIADITIIVPYIGPALNIGNMLSKGEFVEAIIFTGVVAMLEFIPEYALPVFGTF |
| | | AIVSYIANKVLTVQTINNALSKRNEKWDEVYKYTVTNWLAKVNTQIDLIREKMK |
| | | KALENQAEATKAIINYQYNQYTEEEKNNINFNIDDLSSKLNESINSAMININKFLDQ |
| | | CSVSYLMNSMIPYAVKRLKDFDASVRDVLLKYIYDNRGTLVLQVDRLKDEVNNT |
| | | LSADIPFQLSKYVDNKKLLSTFTEYIKNIVNTSILSIVYKKDDLIDLSRYGAKINIGD |
| | | RVYYDSIDKNQIKLINLESSTIEVILKNAIVYNSMYENFSTSFWIKIPKYFSKINLNN |
| | | EYTIINCIENNSGWKVSLNYGEIIWTLQDNKQNIQRVVFKYSQMVNISDYINRWIF |
| | | VTITNNRLTKSKIYINGRLIDQKPISNLGNIHASNKIMFKLDGCRDPRRYIMIKYFNL |
| | | FDKELNEKEIKDLYDSQSNSGILKDFWGNYLQYDKPYYMLNLFDPNKYVDVNNI |
| | | GIRGYMYLKGPRGSVVTTNIYLNSTLYEGTKFIIKKYASGNEDNIVRNNDRVYINV |
| | | VVKNKEYRLATNASQAGVEKILSALEIPDVGNLSQVVVMKSKDDQGIRNKCKMN |
| | | LQDNNGNDIGFIGFHLYDNIAKLVASNWYNRQVGKASRTFGCSWEFIPVDDGWG |
| | | ERSL |
| 17 | BONT/A2- | MPFVNKQFNYKDPVNGVDIAYIKIPNAGQMQPVKAFKIHNKIWVIPERDTFTNPEE |
| | S1295P | GDLNPPPEAKQVPVSYYDSTYLSTDNEKDNYLKGVTKLFERIYSTDLGRMLLTSIV |
| | | RGIPFWGGSTIDTELKVIDTNCINVIQPDGSYRSEELNLVIIGPSADIIQFECKSFGHD |
| | | VLNLTRNGYGSTQYIRFSPDFTFGFEESLEVDTNPLLGAGKFATDPAVTLAHELIH |
| | | AEHRLYGIAINPNRVFKVNTNAYYEMSGLEVSFEELRTFGGHDAKFIDSLQENEFR |
| | | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSLDEGYNKALN |
| | | DLCIKVNNWDLFFSPSEDNFTNDLDKVEEITADTNIEAAEENISLDLIQQYYLTFDF |
| | | DNEPENISIENLSSDIIGQLEPMPNIERFPNGKKYELDKYTMFHYLRAQEFEHGDSRI |
| | | ILTNSAEEALLKPNVAYTFFSSKYVKKINKAVEAFMFLNWAEELVYDFTDETNEV |
| | | TTMDKIADITIIVPYIGPALNIGNMLSKGEFVEAIIFTGVVAMLEFIPEYALPVFGTF |
| | | AIVSYIANKVLTVQTINNALSKRNEKWDEVYKYTVTNWLAKVNTQIDLIREKMK |
| | | KALENQAEATKAIINYQYNQYTEEEKNNINFNIDDLSSKLNESINSAMININKFLDQ |
| | | CSVSYLMNSMIPYAVKRLKDFDASVRDVLLKYIYDNRGTLVLQVDRLKDEVNNT |
| | | LSADIPFQLSKYVDNKKLLSTFTEYIKNIVNTSILSIVYKKDDLIDLSRYGAKINIGD |
| | | RVYYDSIDKNQIKLINLESSTIEVILKNAIVYNSMYENFSTSFWIKIPKYFSKINLNN |
| | | EYTIINCIENNSGWKVSLNYGEIIWTLQDNKQNIQRVVFKYSQMVNISDYINRWIF |
| | | VTITNNRLTKSKIYINGRLIDQKPISNLGNIHASNKIMFKLDGCRDPRRYIMIKYFNL |
| | | FDKELNEKEIKDLYDSQSNSGILKDFWGNYLQYDKPYYMLNLFDPNKYVDVNNI |
| | | GIRGYMYLKGPRGSVVTTNIYLNSTLYEGTKFIIKKYASGNEDNIVRNNDRVYINV |
| | | VVKNKEYRLATNASQAGVEKILSALEIPDVGNLSQVVVMKSKDDQGIRNKCKMN |
| | | LQDNNGNDIGFIGFHLYDNIAKLVASNWYNRQVGKASRTFGCSWEFIPVDDGWG |
| | | ESPL |
| 18 | BONT/A2- | MPFVNKQFNYKDPVNGVDIAYIKIPNAGQMQPVKAFKIHNKIWVIPERDTFTNPEE |
| | S1294R- | GDLNPPPEAKQVPVSYYDSTYLSTDNEKDNYLKGVTKLFERIYSTDLGRMLLTSIV |
| | S1295P | RGIPFWGGSTIDTELKVIDTNCINVIQPDGSYRSEELNLVIIGPSADIIQFECKSFGHD |
| | | VLNLTRNGYGSTQYIRFSPDFTFGFEESLEVDTNPLLGAGKFATDPAVTLAHELIH |
| | | AEHRLYGIAINPNRVFKVNTNAYYEMSGLEVSFEELRTFGGHDAKFIDSLQENEFR |
| | | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSLDEGYNKALN |
| | | DLCIKVNNWDLFFSPSEDNFTNDLDKVEEITADTNIEAAEENISLDLIQQYYLTFDF |
| | | DNEPENISIENLSSDIIGQLEPMPNIERFPNGKKYELDKYTMFHYLRAQEFEHGDSRI |
| | | ILTNSAEEALLKPNVAYTFFSSKYVKKINKAVEAFMFLNWAEELVYDFTDETNEV |
| | | TTMDKIADITIIVPYIGPALNIGNMLSKGEFVEAIIFTGVVAMLEFIPEYALPVFGTF |
| | | AIVSYIANKVLTVQTINNALSKRNEKWDEVYKYTVTNWLAKVNTQIDLIREKMK |
| | | KALENQAEATKAIINYQYNQYTEEEKNNINFNIDDLSSKLNESINSAMININKFLDQ |
| | | CSVSYLMNSMIPYAVKRLKDFDASVRDVLLKYIYDNRGTLVLQVDRLKDEVNNT |
| | | LSADIPFQLSKYVDNKKLLSTFTEYIKNIVNTSILSIVYKKDDLIDLSRYGAKINIGD |
| | | RVYYDSIDKNQIKLINLESSTIEVILKNAIVYNSMYENFSTSFWIKIPKYFSKINLNN |
| | | EYTIINCIENNSGWKVSLNYGEIIWTLQDNKQNIQRVVFKYSQMVNISDYINRWIF |
| | | VTITNNRLTKSKIYINGRLIDQKPISNLGNIHASNKIMFKLDGCRDPRRYIMIKYFNL |
| | | FDKELNEKEIKDLYDSQSNSGILKDFWGNYLQYDKPYYMLNLFDPNKYVDVNNI |
| | | GIRGYMYLKGPRGSVVTTNIYLNSTLYEGTKFIIKKYASGNEDNIVRNNDRVYINV |
| | | VVKNKEYRLATNASQAGVEKILSALEIPDVGNLSQVVVMKSKDDQGIRNKCKMN |
| | | LQDNNGNDIGFIGFHLYDNIAKLVASNWYNRQVGKASRTFGCSWEFIPVDDGWG |
| | | ERPL |
| 19 | BONT/A2- | MPFVNKQFNYKDPVNGVDIAYIKIPNAGQMQPVKAFKIHNKIWVIPERDTFTNPEE |
| | R1064W- | GDLNPPPEAKQVPVSYYDSTYLSTDNEKDNYLKGVTKLFERIYSTDLGRMLLTSIV |
| | S1294R- | RGIPFWGGSTIDTELKVIDTNCINVIQPDGSYRSEELNLVIIGPSADIIQFECKSFGHD |
| | S1295P | VLNLTRNGYGSTQYIRFSPDFTFGFEESLEVDTNPLLGAGKFATDPAVTLAHELIH |
| | | AEHRLYGIAINPNRVFKVNTNAYYEMSGLEVSFEELRTFGGHDAKFIDSLQENEFR |
| | | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSLDEGYNKALN |
| | | DLCIKVNNWDLFFSPSEDNFTNDLDKVEEITADTNIEAAEENISLDLIQQYYLTFDF |
| | | DNEPENISIENLSSDIIGQLEPMPNIERFPNGKKYELDKYTMFHYLRAQEFEHGDSRI |
| | | ILTNSAEEALLKPNVAYTFFSSKYVKKINKAVEAFMFLNWAEELVYDFTDETNEV |
| | | TTMDKIADITIIVPYIGPALNIGNMLSKGEFVEAIIFTGVVAMLEFIPEYALPVFGTF |
| | | AIVSYIANKVLTVQTINNALSKRNEKWDEVYKYTVTNWLAKVNTQIDLIREKMK |
| | | KALENQAEATKAIINYQYNQYTEEEKNNINFNIDDLSSKLNESINSAMININKFLDQ |
| | | CSVSYLMNSMIPYAVKRLKDFDASVRDVLLKYIYDNRGTLVLQVDRLKDEVNNT |
| | | LSADIPFQLSKYVDNKKLLSTFTEYIKNIVNTSILSIVYKKDDLIDLSRYGAKINIGD |
| | | RVYYDSIDKNQIKLINLESSTIEVILKNAIVYNSMYENFSTSFWIKIPKYFSKINLNN |
| | | EYTIINCIENNSGWKVSLNYGEIIWTLQDNKQNIQRVVFKYSQMVNISDYINRWIF |
| | | VTITNNRLTKSKIYINGRLIDQKPISNLGNIHASNKIMFKLDGCRDPWRYIMIKYFN |
| | | LFDKELNEKEIKDLYDSQSNSGILKDFWGNYLQYDKPYYMLNLFDPNKYVDVNN |
| | | IGIRGYMYLKGPRGSVVTTNIYLNSTLYEGTKFIIKKYASGNEDNIVRNNDRVYIN |
| | | VVVKNKEYRLATNASQAGVEKILSALEIPDVGNLSQVVVMKSKDDQGIRNKCKM |
| | | NLQDNNGNDIGFIGFHLYDNIAKLVASNWYNRQVGKASRTFGCSWEFIPVDDGW |
| | | GERPL |
| 20 | BONT/A2- | MPFVNKQFNYKDPVNGVDIAYIKIPNAGQMQPVKAFKIHNKIWVIPERDTFTNPEE |
| | R1064F- | GDLNPPPEAKQVPVSYYDSTYLSTDNEKDNYLKGVTKLFERIYSTDLGRMLLTSIV |
| | S1294R- | RGIPFWGGSTIDTELKVIDTNCINVIQPDGSYRSEELNLVIIGPSADIIQFECKSFGHD |
| | S1295P | VLNLTRNGYGSTQYIRFSPDFTFGFEESLEVDTNPLLGAGKFATDPAVTLAHELIH |
| | | AEHRLYGIAINPNRVFKVNTNAYYEMSGLEVSFEELRTFGGHDAKFIDSLQENEFR |
| | | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSLDEGYNKALN |
| | | DLCIKVNNWDLFFSPSEDNFTNDLDKVEEITADTNIEAAEENISLDLIQQYYLTFDF |
| | | DNEPENISIENLSSDIIGQLEPMPNIERFPNGKKYELDKYTMFHYLRAQEFEHGDSRI |
| | | ILTNSAEEALLKPNVAYTFFSSKYVKKINKAVEAFMFLNWAEELVYDFTDETNEV |
| | | TTMDKIADITIIVPYIGPALNIGNMLSKGEFVEAIIFTGVVAMLEFIPEYALPVFGTF |
| | | AIVSYIANKVLTVQTINNALSKRNEKWDEVYKYTVTNWLAKVNTQIDLIREKMK |
| | | KALENQAEATKAIINYQYNQYTEEEKNNINFNIDDLSSKLNESINSAMININKFLDQ |
| | | CSVSYLMNSMIPYAVKRLKDFDASVRDVLLKYIYDNRGTLVLQVDRLKDEVNNT |
| | | LSADIPFQLSKYVDNKKLLSTFTEYIKNIVNTSILSIVYKKDDLIDLSRYGAKINIGD |
| | | RVYYDSIDKNQIKLINLESSTIEVILKNAIVYNSMYENFSTSFWIKIPKYFSKINLNN |
| | | EYTIINCIENNSGWKVSLNYGEIIWTLQDNKQNIQRVVFKYSQMVNISDYINRWIF |
| | | VTITNNRLTKSKIYINGRLIDQKPISNLGNIHASNKIMFKLDGCRDPFRYIMIKYFNL |
| | | FDKELNEKEIKDLYDSQSNSGILKDFWGNYLQYDKPYYMLNLFDPNKYVDVNNI |
| | | GIRGYMYLKGPRGSVVTTNIYLNSTLYEGTKFIIKKYASGNEDNIVRNNDRVYINV |
| | | VVKNKEYRLATNASQAGVEKILSALEIPDVGNLSQVVVMKSKDDQGIRNKCKMN |
| | | LQDNNGNDIGFIGFHLYDNIAKLVASNWYNRQVGKASRTFGCSWEFIPVDDGWG |
| | | ERPL |
| 21 | BONT/A2- | MPFVNKQFNYKDPVNGVDIAYIKIPNAGQMQPVKAFKIHNKIWVIPERDTFTNPEE |
| | R1064H- | GDLNPPPEAKQVPVSYYDSTYLSTDNEKDNYLKGVTKLFERIYSTDLGRMLLTSIV |
| | S1294R- | RGIPFWGGSTIDTELKVIDTNCINVIQPDGSYRSEELNLVIIGPSADIIQFECKSFGHD |
| | S1295P | VLNLTRNGYGSTQYIRFSPDFTFGFEESLEVDTNPLLGAGKFATDPAVTLAHELIH |
| | | AEHRLYGIAINPNRVFKVNTNAYYEMSGLEVSFEELRTFGGHDAKFIDSLQENEFR |
| | | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSLDEGYNKALN |
| | | DLCIKVNNWDLFFSPSEDNFTNDLDKVEEITADTNIEAAEENISLDLIQQYYLTFDF |
| | | DNEPENISIENLSSDIIGQLEPMPNIERFPNGKKYELDKYTMFHYLRAQEFEHGDSRI |
| | | ILTNSAEEALLKPNVAYTFFSSKYVKKINKAVEAFMFLNWAEELVYDFTDETNEV |
| | | TTMDKIADITIIVPYIGPALNIGNMLSKGEFVEAIIFTGVVAMLEFIPEYALPVFGTF |
| | | AIVSYIANKVLTVQTINNALSKRNEKWDEVYKYTVTNWLAKVNTQIDLIREKMK |
| | | KALENQAEATKAIINYQYNQYTEEEKNNINFNIDDLSSKLNESINSAMININKFLDQ |
| | | CSVSYLMNSMIPYAVKRLKDFDASVRDVLLKYIYDNRGTLVLQVDRLKDEVNNT |
| | | LSADIPFQLSKYVDNKKLLSTFTEYIKNIVNTSILSIVYKKDDLIDLSRYGAKINIGD |
| | | RVYYDSIDKNQIKLINLESSTIEVILKNAIVYNSMYENFSTSFWIKIPKYFSKINLNN |
| | | EYTIINCIENNSGWKVSLNYGEIIWTLQDNKQNIQRVVFKYSQMVNISDYINRWIF |
| | | VTITNNRLTKSKIYINGRLIDQKPISNLGNIHASNKIMFKLDGCRDPHRYIMIKYFNL |
| | | FDKELNEKEIKDLYDSQSNSGILKDFWGNYLQYDKPYYMLNLFDPNKYVDVNNI |
| | | GIRGYMYLKGPRGSVVTTNIYLNSTLYEGTKFIIKKYASGNEDNIVRNNDRVYINV |
| | | VVKNKEYRLATNASQAGVEKILSALEIPDVGNLSQVVVMKSKDDQGIRNKCKMN |
| | | LQDNNGNDIGFIGFHLYDNIAKLVASNWYNRQVGKASRTFGCSWEFIPVDDGWG |
| | | ERPL |
| 22 | BONT/A2- | MPFVNKQFNYKDPVNGVDIAYIKIPNAGQMQPVKAFKIHNKIWVIPERDTFTNPEE |
| | R1064L- | GDLNPPPEAKQVPVSYYDSTYLSTDNEKDNYLKGVTKLFERIYSTDLGRMLLTSIV |
| | S1294R- | RGIPFWGGSTIDTELKVIDTNCINVIQPDGSYRSEELNLVIIGPSADIIQFECKSFGHD |
| | S1295P | VLNLTRNGYGSTQYIRFSPDFTFGFEESLEVDTNPLLGAGKFATDPAVTLAHELIH |
| | | AEHRLYGIAINPNRVFKVNTNAYYEMSGLEVSFEELRTFGGHDAKFIDSLQENEFR |
| | | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSLDEGYNKALN |
| | | DLCIKVNNWDLFFSPSEDNFTNDLDKVEEITADTNIEAAEENISLDLIQQYYLTFDF |
| | | DNEPENISIENLSSDIIGQLEPMPNIERFPNGKKYELDKYTMFHYLRAQEFEHGDSRI |
| | | ILTNSAEEALLKPNVAYTFFSSKYVKKINKAVEAFMFLNWAEELVYDFTDETNEV |
| | | TTMDKIADITIIVPYIGPALNIGNMLSKGEFVEAIIFTGVVAMLEFIPEYALPVFGTF |
| | | AIVSYIANKVLTVQTINNALSKRNEKWDEVYKYTVTNWLAKVNTQIDLIREKMK |
| | | KALENQAEATKAIINYQYNQYTEEEKNNINFNIDDLSSKLNESINSAMININKFLDQ |
| | | CSVSYLMNSMIPYAVKRLKDFDASVRDVLLKYIYDNRGTLVLQVDRLKDEVNNT |
| | | LSADIPFQLSKYVDNKKLLSTFTEYIKNIVNTSILSIVYKKDDLIDLSRYGAKINIGD |
| | | RVYYDSIDKNQIKLINLESSTIEVILKNAIVYNSMYENFSTSFWIKIPKYFSKINLNN |
| | | EYTIINCIENNSGWKVSLNYGEIIWTLQDNKQNIQRVVFKYSQMVNISDYINRWIF |
| | | VTITNNRLTKSKIYINGRLIDQKPISNLGNIHASNKIMFKLDGCRDPLRYIMIKYFNL |
| | | FDKELNEKEIKDLYDSQSNSGILKDFWGNYLQYDKPYYMLNLFDPNKYVDVNNI |
| | | GIRGYMYLKGPRGSVVTTNIYLNSTLYEGTKFIIKKYASGNEDNIVRNNDRVYINV |
| | | VVKNKEYRLATNASQAGVEKILSALEIPDVGNLSQVVVMKSKDDQGIRNKCKMN |
| | | LQDNNGNDIGFIGFHLYDNIAKLVASNWYNRQVGKASRTFGCSWEFIPVDDGWG |
| | | ERPL |
| 23 | HC-BoNT/A2 | DLIDLSRYGAKINIGDRVYYDSIDKNQIKLINLESSTIEVILKNAIVYNSMYENFSTS |
| | wildtype | FWIKIPKYFSKINLNNEYTIINCIENNSGWKVSLNYGEIIWTLQDNKQNIQRVVFKY |
| | | SQMVNISDYINRWIFVTITNNRLTKSKIYINGRLIDQKPISNLGNIHASNKIMFKLDG |
| | | CRDPRRYIMIKYFNLFDKELNEKEIKDLYDSQSNSGILKDFWGNYLQYDKPYYML |
| | | NLFDPNKYVDVNNIGIRGYMYLKGPRGSVVTTNIYLNSTLYEGTKFIIKKYASGNE |
| | | DNIVRNNDRVYINVVVKNKEYRLATNASQAGVEKILSALEIPDVGNLSQVVVMKS |
| | | KDDQGIRNKCKMNLQDNNGNDIGFIGFHLYDNIAKLVASNWYNRQVGKASRTFG |
| | | CSWEFIPVDDGWGESSL |
| 24 | HC- | DLIDLSRYGAKINIGDRVYYDSIDKNQIKLINLESSTIEVILKNAIVYNSMYENFSTS |
| | BONT/A2- | FWIKIPKYFSKINLNNEYTIINCIENNSGWKVSLNYGEIIWTLQDNKQNIQRVVFKY |
| | R1064W | SQMVNISDYINRWIFVTITNNRLTKSKIYINGRLIDQKPISNLGNIHASNKIMFKLDG |
| | | CRDPWRYIMIKYFNLFDKELNEKEIKDLYDSQSNSGILKDFWGNYLQYDKPYYML |
| | | NLFDPNKYVDVNNIGIRGYMYLKGPRGSVVTTNIYLNSTLYEGTKFIIKKYASGNE |
| | | DNIVRNNDRVYINVVVKNKEYRLATNASQAGVEKILSALEIPDVGNLSQVVVMKS |
| | | KDDQGIRNKCKMNLQDNNGNDIGFIGFHLYDNIAKLVASNWYNRQVGKASRTFG |
| | | CSWEFIPVDDGWGESSL |
| 25 | HC- | DLIDLSRYGAKINIGDRVYYDSIDKNQIKLINLESSTIEVILKNAIVYNSMYENFSTS |
| | BONT/A2- | FWIKIPKYFSKINLNNEYTIINCIENNSGWKVSLNYGEIIWTLQDNKQNIQRVVFKY |
| | R1064F | SQMVNISDYINRWIFVTITNNRLTKSKIYINGRLIDQKPISNLGNIHASNKIMFKLDG |
| | | CRDPFRYIMIKYFNLFDKELNEKEIKDLYDSQSNSGILKDFWGNYLQYDKPYYML |
| | | NLFDPNKYVDVNNIGIRGYMYLKGPRGSVVTTNIYLNSTLYEGTKFIIKKYASGNE |
| | | DNIVRNNDRVYINVVVKNKEYRLATNASQAGVEKILSALEIPDVGNLSQVVVMKS |
| | | KDDQGIRNKCKMNLQDNNGNDIGFIGFHLYDNIAKLVASNWYNRQVGKASRTFG |
| | | CSWEFIPVDDGWGESSL |
| 26 | HC- | DLIDLSRYGAKINIGDRVYYDSIDKNQIKLINLESSTIEVILKNAIVYNSMYENFSTS |
| | BONT/A2- | FWIKIPKYFSKINLNNEYTIINCIENNSGWKVSLNYGEIIWTLQDNKQNIQRVVFKY |
| | R1064H | SQMVNISDYINRWIFVTITNNRLTKSKIYINGRLIDQKPISNLGNIHASNKIMFKLDG |
| | | CRDPHRYIMIKYFNLFDKELNEKEIKDLYDSQSNSGILKDFWGNYLQYDKPYYML |
| | | NLFDPNKYVDVNNIGIRGYMYLKGPRGSVVTTNIYLNSTLYEGTKFIIKKYASGNE |
| | | DNIVRNNDRVYINVVVKNKEYRLATNASQAGVEKILSALEIPDVGNLSQVVVMKS |
| | | KDDQGIRNKCKMNLQDNNGNDIGFIGFHLYDNIAKLVASNWYNRQVGKASRTFG |
| | | CSWEFIPVDDGWGESSL |
| 27 | HC- | DLIDLSRYGAKINIGDRVYYDSIDKNQIKLINLESSTIEVILKNAIVYNSMYENFSTS |
| | BONT/A2- | FWIKIPKYFSKINLNNEYTIINCIENNSGWKVSLNYGEIIWTLQDNKQNIQRVVFKY |
| | R1064L | SQMVNISDYINRWIFVTITNNRLTKSKIYINGRLIDQKPISNLGNIHASNKIMFKLDG |
| | | CRDPLRYIMIKYFNLFDKELNEKEIKDLYDSQSNSGILKDFWGNYLQYDKPYYML |
| | | NLFDPNKYVDVNNIGIRGYMYLKGPRGSVVTTNIYLNSTLYEGTKFIIKKYASGNE |
| | | DNIVRNNDRVYINVVVKNKEYRLATNASQAGVEKILSALEIPDVGNLSQVVVMKS |
| | | KDDQGIRNKCKMNLQDNNGNDIGFIGFHLYDNIAKLVASNWYNRQVGKASRTFG |
| | | CSWEFIPVDDGWGESSL |
| 28 | HC- | DLIDLSRYGAKINIGDRVYYDSIDKNQIKLINLESSTIEVILKNAIVYNSMYENFSTS |
| | BoNT/A2- | FWIKIPKYFSKINLNNEYTIINCIENNSGWKVSLNYGEIIWTLQDNKQNIQRVVFKY |
| | S1294R | SQMVNISDYINRWIFVTITNNRLTKSKIYINGRLIDQKPISNLGNIHASNKIMFKLDG |
| | | CRDPRRYIMIKYFNLFDKELNEKEIKDLYDSQSNSGILKDFWGNYLQYDKPYYML |
| | | NLFDPNKYVDVNNIGIRGYMYLKGPRGSVVTTNIYLNSTLYEGTKFIIKKYASGNE |
| | | DNIVRNNDRVYINVVVKNKEYRLATNASQAGVEKILSALEIPDVGNLSQVVVMKS |
| | | KDDQGIRNKCKMNLQDNNGNDIGFIGFHLYDNIAKLVASNWYNRQVGKASRTFG |
| | | CSWEFIPVDDGWGERSL |
| 29 | HC- | DLIDLSRYGAKINIGDRVYYDSIDKNQIKLINLESSTIEVILKNAIVYNSMYENFSTS |
| | BONT/A2- | FWIKIPKYFSKINLNNEYTIINCIENNSGWKVSLNYGEIIWTLQDNKQNIQRVVFKY |
| | S1295P | SQMVNISDYINRWIFVTITNNRLTKSKIYINGRLIDQKPISNLGNIHASNKIMFKLDG |
| | | CRDPRRYIMIKYFNLFDKELNEKEIKDLYDSQSNSGILKDFWGNYLQYDKPYYML |
| | | NLFDPNKYVDVNNIGIRGYMYLKGPRGSVVTTNIYLNSTLYEGTKFIIKKYASGNE |
| | | DNIVRNNDRVYINVVVKNKEYRLATNASQAGVEKILSALEIPDVGNLSQVVVMKS |
| | | KDDQGIRNKCKMNLQDNNGNDIGFIGFHLYDNIAKLVASNWYNRQVGKASRTFG |
| | | CSWEFIPVDDGWGESPL |
| 30 | HC- | DLIDLSRYGAKINIGDRVYYDSIDKNQIKLINLESSTIEVILKNAIVYNSMYENFSTS |
| | BoNT/A2- | FWIKIPKYFSKINLNNEYTIINCIENNSGWKVSLNYGEIIWTLQDNKQNIQRVVFKY |
| | S1294R- | SQMVNISDYINRWIFVTITNNRLTKSKIYINGRLIDQKPISNLGNIHASNKIMFKLDG |
| | S1295P | CRDPRRYIMIKYFNLFDKELNEKEIKDLYDSQSNSGILKDFWGNYLQYDKPYYML |
| | | NLFDPNKYVDVNNIGIRGYMYLKGPRGSVVTTNIYLNSTLYEGTKFIIKKYASGNE |
| | | DNIVRNNDRVYINVVVKNKEYRLATNASQAGVEKILSALEIPDVGNLSQVVVMKS |
| | | KDDQGIRNKCKMNLQDNNGNDIGFIGFHLYDNIAKLVASNWYNRQVGKASRTFG |
| | | CSWEFIPVDDGWGERPL |
| 31 | HC- | DLIDLSRYGAKINIGDRVYYDSIDKNQIKLINLESSTIEVILKNAIVYNSMYENFSTS |
| | BONT/A2- | FWIKIPKYFSKINLNNEYTIINCIENNSGWKVSLNYGEIIWTLQDNKQNIQRVVFKY |
| | R1064W- | SQMVNISDYINRWIFVTITNNRLTKSKIYINGRLIDQKPISNLGNIHASNKIMFKLDG |
| | S1294R- | CRDPWRYIMIKYFNLFDKELNEKEIKDLYDSQSNSGILKDFWGNYLQYDKPYYML |
| | S1295P | NLFDPNKYVDVNNIGIRGYMYLKGPRGSVVTTNIYLNSTLYEGTKFIIKKYASGNE |
| | | DNIVRNNDRVYINVVVKNKEYRLATNASQAGVEKILSALEIPDVGNLSQVVVMKS |
| | | KDDQGIRNKCKMNLQDNNGNDIGFIGFHLYDNIAKLVASNWYNRQVGKASRTFG |
| | | CSWEFIPVDDGWGERPL |
| 32 | HC- | DLIDLSRYGAKINIGDRVYYDSIDKNQIKLINLESSTIEVILKNAIVYNSMYENFSTS |
| | BONT/A2- | FWIKIPKYFSKINLNNEYTIINCIENNSGWKVSLNYGEIIWTLQDNKQNIQRVVFKY |
| | R1064F- | SQMVNISDYINRWIFVTITNNRLTKSKIYINGRLIDQKPISNLGNIHASNKIMFKLDG |
| | S1294R- | CRDPFRYIMIKYFNLFDKELNEKEIKDLYDSQSNSGILKDFWGNYLQYDKPYYML |
| | S1295P | NLFDPNKYVDVNNIGIRGYMYLKGPRGSVVTTNIYLNSTLYEGTKFIIKKYASGNE |
| | | DNIVRNNDRVYINVVVKNKEYRLATNASQAGVEKILSALEIPDVGNLSQVVVMKS |
| | | KDDQGIRNKCKMNLQDNNGNDIGFIGFHLYDNIAKLVASNWYNRQVGKASRTFG |
| | | CSWEFIPVDDGWGERPL |
| 33 | HC- | DLIDLSRYGAKINIGDRVYYDSIDKNQIKLINLESSTIEVILKNAIVYNSMYENFSTS |
| | BoNT/A2- | FWIKIPKYFSKINLNNEYTIINCIENNSGWKVSLNYGEIIWTLQDNKQNIQRVVFKY |
| | R1064H- | SQMVNISDYINRWIFVTITNNRLTKSKIYINGRLIDQKPISNLGNIHASNKIMFKLDG |
| | S1294R- | CRDPHRYIMIKYFNLFDKELNEKEIKDLYDSQSNSGILKDFWGNYLQYDKPYYML |
| | S1295P | NLFDPNKYVDVNNIGIRGYMYLKGPRGSVVTTNIYLNSTLYEGTKFIIKKYASGNE |
| | | DNIVRNNDRVYINVVVKNKEYRLATNASQAGVEKILSALEIPDVGNLSQVVVMKS |
| | | KDDQGIRNKCKMNLQDNNGNDIGFIGFHLYDNIAKLVASNWYNRQVGKASRTFG |
| | | CSWEFIPVDDGWGERPL |
| 34 | HC- | DLIDLSRYGAKINIGDRVYYDSIDKNQIKLINLESSTIEVILKNAIVYNSMYENFSTS |
| | BONT/A2- | FWIKIPKYFSKINLNNEYTIINCIENNSGWKVSLNYGEIIWTLQDNKQNIQRVVFKY |
| | R1064L- | SQMVNISDYINRWIFVTITNNRLTKSKIYINGRLIDQKPISNLGNIHASNKIMFKLDG |
| | S1294R- | CRDPLRYIMIKYFNLFDKELNEKEIKDLYDSQSNSGILKDFWGNYLQYDKPYYML |
| | S1295P | NLFDPNKYVDVNNIGIRGYMYLKGPRGSVVTTNIYLNSTLYEGTKFIIKKYASGNE |
| | | DNIVRNNDRVYINVVVKNKEYRLATNASQAGVEKILSALEIPDVGNLSQVVVMKS |
| | | KDDQGIRNKCKMNLQDNNGNDIGFIGFHLYDNIAKLVASNWYNRQVGKASRTFG |
| | | CSWEFIPVDDGWGERPL |
| 35 | BONT/B | MPVTINNFNYNDPIDNNNIIMMEPPFARGTGRYYKAFKITDRIWIIPERYTFGYKPE |
| | protease and | DFNKSSGIFNRDVCEYYDPDYLNTNDKKNIFLQTMIKLFNRIKSKPLGEKLLEMIIN |
| | translocation | GIPYLGDRRVPLEEFNTNIASVTVNKLISNPGEVERKKGIFANLIIFGPGPVLNENET |
| | domain (AA | IDIGIQNHFASREGFGGIMQMKFCPEYVSVENNVQENKGASIFNRRGYFSDPALIL |
| | 1-857) | MHELIHVLHGLYGIKVDDLPIVPNEKKFFMQSTDAIQAEELYTFGGQDPSIITPSTD |
| | | KSIYDKVLQNFRGIVDRLNKVLVCISDPNININIYKNKFKDKYKFVEDSEGKYSIDV |
| | | ESFDKLYKSLMFGFTETNIAENYKIKTRASYFSDSLPPVKIKNLLDNEIYTIEEGFNI |
| | | SDKDMEKEYRGQNKAINKQAYEEISKEHLAVYKIQMCKSVKAPGICIDVDNEDLF |
| | | FIADKNSFSDDLSKNERIEYNTQSNYIENDFPINELILDTDLISKIELPSENTESLTDF |
| | | NVDVPVYEKQPAIKKIFTDENTIFQYLYSQTFPLDIRDISLTSSFDDALLFSNKVYSF |
| | | FSMDYIKTANKVVEAGLFAGWVKQIVNDFVIEANKSNTMDKIADISLIVPYIGLAL |
| | | NVGNETAKGNFENAFEIAGASILLEFIPELLIPVVGAFLLESYIDNKNKIIKTIDNALT |
| | | KRNEKWSDMYGLIVAQWLSTVNTQFYTIKEGMYKALNYQAQALEEIIKYRYNIY |
| | | SEKEKSNINIDENDINSKLNEGINQAIDNINNFINGCSVSYLMKKMIPLAVEKLLDF |
| | | DNTLKKNLLNYIDENKLYLIGSAEYEKSKVNKYLKTIMPFDLSIYTNDTILIEMFNK |
| | | YN |
| 36 | BONT/C | MPITINNFNYSDPVDNKNILYLDTHLNTLANEPEKAFRITGNIWVIPDRESRNSNPN |
| | protease and | LNKPPRVTSPKSGYYDPNYLSTDSDKDTFLKEIIKLFKRINSREIGEELIYRLSTDIPF |
| | translocation | PGNNNTPINTFDFDVDFNSVDVKTRQGNNWVKTGSINPSVIITGPRENIIDPETSTF |
| | domain (AA | KLTNNTFAAQEGFGALSIISISPRFMLTYSNATNDVGEGRFSKSEFCMDPILILMHE |
| | 1-870) | LNHAMHNLYGIAIPNDQTISSVTSNIFYSQYNVKLEYAEIYAFGGPTIDLIPKSARK |
| | | YFEEKALDYYRSIAKRLNSITTANPSSFNKYIGEYKQKLIRKYRFVVESSGEVTVNR |
| | | NKFVELYNELTQIFTEFNYAKIYNVQNRKIYLSNVYTPVTANILDDNVYDIQNGEN |
| | | IPKSNLNVLFMGQNLSRNPALRKVNPENMLYLFTKFCHKAIDGRSLYNKTLDCRE |
| | | LLVKNTDLPFIGDISDVKTDIFLRKDINEETEVIYYPDNVSVDQVILSKNTSEHGQL |
| | | DLLYPSIDSESEILPGENQVFYDNRTQNVDYLNSYYYLESQKLSDNVEDFTFTRSIE |
| | | EALDNSAKVYTYFPTLANKVNAGVQGGLFLMWANDVVEDFTTNILRKDTLDKIS |
| | | DVSAIIPYIGPALNISNSVRRGNFTEAFAVTGVTILLEAFPEFTIPALGAFVIYSKVQE |
| | | RNEIIKTIDNCLEQRIKRWKDSYEWMMGTWLSRIITQFNNISYQMYDSLNYQAGAI |
| | | KAKIDLEYKKYSGSDKENIKSQVENLKNSLDVKISEAMNNINKFIRECSVTYLFKN |
| | | MLPKVIDELNEFDRNTKAKLINLIDSHNIILVGEVDKLKAKVNNSFQNTIPFNIFSYT |
| | | NNSLLKDIINEYFNNIND |
| 37 | BONT/D | MTWPVKDFNYSDPVNDNDILYLRIPQNKLITTPVKAFMITQNIWVIPERFSSDTNPS |
| | protease and | LSKPPRPTSKYQSYYDPSYLSTDEQKDTFLKGIIKLFKRINERDIGKKLINYLVVGSP |
| | translocation | FMGDSSTPEDTFDFTRHTTNIAVEKFENGSWKVTNIITPSVLIFGPLPNILDYTASLT |
| | domain (AA | LQGQQSNPSFEGFGTLSILKVAPEFLLTFSDVTSNQSSAVLGKSIFCMDPVIALMHE |
| | 1-862) | LTHSLHQLYGINIPSDKRIRPQVSEGFFSQDGPNVQFEELYTFGGLDVEIIPQIERSQ |
| | | LREKALGHYKDIAKRLNNINKTIPSSWISNIDKYKKIFSEKYNFDKDNTGNFVVNID |
| | | KFNSLYSDLTNVMSEVVYSSQYNVKNRTHYFSRHYLPVFANILDDNIYTIRDGFNL |
| | | TNKGFNIENSGQNIERNPALQKLSSESVVDLFTKVCLRLTKNSRDDSTCIKVKNNR |
| | | LPYVADKDSISQEIFENKIITDETNVQNYSDNESLDESILDGQVPINPEIVDPLLPNV |
| | | NMEPLNLPGEEIVFYDDITKYVDYLNSYYYLESQKLSNNVENITLTTSVEEALGYS |
| | | NKIYTFLPSLAEKVNKGVQAGLFLNWANEVVEDFTTNIMKKDTLDKISDVSVIIPY |
| | | IGPALNIGNSALRGNFKQAFATAGVAFLLEGFPEFTIPALGVFTFYSSIQEREKIIKTI |
| | | ENCLEQRVKRWKDSYQWMVSNWLSRITTQFNHINYQMYDSLSYQADAIKAKIDL |
| | | EYKKYSGSDKENIKSQVENLKNSLDVKISEAMNNINKFIRECSVTYLFKNMLPKVI |
| | | DELNKFDLRTKTELINLIDSHNIILVGEVDRLKAKVNESFENTMPFNIFSYTNNSLL |
| | | KDIINEYFN |
| 38 | BONT/E | MPKINSFNYNDPVNDRTILYIKPGGCQEFYKSFNIMKNIWIIPERNVIGTTPQDFHPP |
| | protease and | TSLKNGDSSYYDPNYLQSDEEKDRFLKIVTKIFNRINNNLSGGILLEELSKANPYLG |
| | translocation | NDNTPDNQFHIGDASAVEIKFSNGSQDILLPNVIIMGAEPDLFETNSSNISLRNNYM |
| | domain (AA | PSNHRFGSIAIVTFSPEYSFRFNDNCMNEFIQDPALTLMHELIHSLHGLYGAKGITT |
| | 1-844) | KYTITQKQNPLITNIRGTNIEEFLTFGGTDLNIITSAQSNDIYTNLLADYKKIASKLS |
| | | KVQVSNPLLNPYKDVFEAKYGLDKDASGIYSVNINKENDIFKKLYSFTEFDLRTKF |
| | | QVKCRQTYIGQYKYFKLSNLLNDSIYNISEGYNINNLKVNFRGQNANLNPRIITPIT |
| | | GRGLVKKIIRFCKNIVSVKGIRKSICIEINNGELFFVASENSYNDDNINTPKEIDDTV |
| | | TSNNNYENDLDQVILNFNSESAPGLSDEKLNLTIQNDAYIPKYDSNGTSDIEQHDV |
| | | NELNVFFYLDAQKVPEGENNVNLTSSIDTALLEQPKIYTFFSSEFINNVNKPVQAAL |
| | | FVSWIQQVLVDFTTEANQKSTVDKIADISIVVPYIGLALNIGNEAQKGNFKDALEL |
| | | LGAGILLEFEPELLIPTILVFTIKSFLGSSDNKNKVIKAINNALKERDEKWKEVYSFI |
| | | VSNWMTKINTQFNKRKEQMYQALQNQVNAIKTIIESKYNSYTLEEKNELTNKYDI |
| | | KQIENELNQKVSIAMNNIDRFLTESSISYLMKIINEVKINKLREYDENVKTYLLNYII |
| | | QHGSILGESQQELNSMVTDTLNNSIPFKLSSYTDDKILISYFNKFF |
| 39 | BONT/F | MPVVINSFNYNDPVNDDTILYMQIPYEEKSKKYYKAFEIMRNVWIIPERNTIGTDP |
| | protease and | SDFDPPASLENGSSAYYDPNYLTTDAEKDRYLKTTIKLFKRINSNPAGEVLLQEISY |
| | translocation | AKPYLGNEHTPINEFHPVTRTTSVNIKSSTNVKSSIILNLLVLGAGPDIFENSSYPVR |
| | domain (1- | KLMDSGGVYDPSNDGFGSINIVTFSPEYEYTFNDISGGYNSSTESFIADPAISLAHEL |
| | 863) | IHALHGLYGARGVTYKETIKVKQAPLMIAEKPIRLEEFLTFGGQDLNIITSAMKEKI |
| | | YNNLLANYEKIATRLSRVNSAPPEYDINEYKDYFQWKYGLDKNADGSYTVNENK |
| | | FNEIYKKLYSFTEIDLANKFKVKCRNTYFIKYGFLKVPNLLDDDIYTVSEGFNIGNL |
| | | AVNNRGQNIKLNPKIIDSIPDKGLVEKIVKFCKSVIPRKGTKAPPRLCIRVNNRELFF |
| | | VASESSYNENDINTPKEIDDTTNLNNNYRNNLDEVILDYNSETIPQISNQTLNTLVQ |
| | | DDSYVPRYDSNGTSEIEEHNVVDLNVFFYLHAQKVPEGETNISLTSSIDTALSEESQ |
| | | VYTFFSSEFINTINKPVHAALFISWINQVIRDFTTEATQKSTFDKIADISLVVPYVGL |
| | | ALNIGNEVQKENFKEAFELLGAGILLEFVPELLIPTILVFTIKSFIGSSENKNKIIKAIN |
| | | NSLMERETKWKEIYSWIVSNWLTRINTQFNKRKEQMYQALQNQVDAIKTVIEYK |
| | | YNNYTSDERNRLESEYNINNIREELNKKVSLAMENIERFITESSIFYLMKLINEAKV |
| | | SKLREYDEGVKEYLLDYISEHRSILGNSVQELNDLVTSTLNNSIPFELSSYTNDKILI |
| | | LYFNKLY |
| 40 | BONT/H | MPVVINSFNYDDPVNDNTIIYIRPPYYETSNTYFKAFQIMDNVWIIPERYRLGIDPSL |
| | protease and | FNPPVSLKAGSDGYFDPNYLSTNTEKNKYLQIMIKLFKRINSKPAGQILLEEIKNAIP |
| | translocation | YLGNSYTQEEQFTTNNRTVSFNVKLANGNIVQQMANLIIWGPGPDLTTNKTGGIIY |
| | domain (AA | SPYQSMEATPYKDGFGSIMTVEFSPEYATAFNDISIASHSPSLFIKDPALILMHELIH |
| | 1-858) | VLHGLYGTYITEYKITPNVVQSYMKVTKPITSAEFLTFGGRDRNIVPQSIQSQLYNK |
| | | VLSDYKRIASRLNKVNTATALINIDEFKNLYEWKYQFAKDSNGVYSVDLNKFEQL |
| | | YKKIYSFTEFNLAYEFKIKTRLGYLAENFGPFYLPNLLDDSIYTEVDGFNIGALSIN |
| | | YQGQNIGSDINSIKKLQGQGVVSRVVRLCSNSNTKNSLCITVNNRDLFFIASQESY |
| | | GENTINTYKEIDDTTTLDPSFEDILDKVILNFNEQVIPQMPNRNVSTDIQKDNYIPK |
| | | YDYNRTDIIDSYEVGRNYNTFFYLNAQKFSPNESNITLTSSFDTGLLEGSKVYTFFS |
| | | SDFINNINKPVQALLFIEWVKQVIRDFTTEATKTSTVDKLKDISLVVPYIGLALNIG |
| | | DEIYKQHFAEAVELVGAGLLLEFSPEFLIPTLLIFTIKGYLTGSIRDKDKIIKTLDNAL |
| | | NVRDQKWKELYRWVVSKWLTTINTQFNKRKEQMYKALKNQATAIKKIIENKYN |
| | | NYTTDEKSKIDSSYNINEIERTLNEKINLAMKNIEQFITESSIAYLINIINNETIQKLKS |
| | | YDDLVRRYLLGYIRNHSSILGNSVEELNSKVNNHLDNGIPFELSSYTNDSLLIRYFN |
| | | KNY |
| 41 | BONT/X | MKLEINKFNYNDPIDGINVITMRPPRHSDKINKGKGPFKAFQVIKNIWIVPERYNFT |
| | protease and | NNTNDLNIPSEPIMEADAIYNPNYLNTPSEKDEFLQGVIKVLERIKSKPEGEKLLELI |
| | translocation | SSSIPLPLVSNGALTLSDNETIAYQENNNIVSNLQANLVIYGPGPDIANNATYGLYS |
| | domain (AA | TPISNGEGTLSEVSFSPFYLKPFDESYGNYRSLVNIVNKFVKREFAPDPASTLMHEL |
| | 1-889) | VHVTHNLYGISNRNFYYNFDTGKIETSRQQNSLIFEELLTFGGIDSKAISSLIIKKIIE |
| | | TAKNNYTTLISERLNTVTVENDLLKYIKNKIPVQGRLGNFKLDTAEFEKKLNTILF |
| | | VLNESNLAQRFSILVRKHYLKERPIDPIYVNILDDNSYSTLEGFNISSQGSNDFQGQ |
| | | LLESSYFEKIESNALRAFIKICPRNGLLYNAIYRNSKNYLNNIDLEDKKTTSKTNVS |
| | | YPCSLLNGCIEVENKDLFLISNKDSLNDINLSEEKIKPETTVFFKDKLPPQDITLSNY |
| | | DFTEANSIPSISQQNILERNEELYEPIRNSLFEIKTIYVDKLTTFHFLEAQNIDESIDSS |
| | | KIRVELTDSVDEALSNPNKVYSPFKNMSNTINSIETGITSTYIFYQWLRSIVKDFSDE |
| | | TGKIDVIDKSSDTLAIVPYIGPLLNIGNDIRHGDFVGAIELAGITALLEYVPEFTIPIL |
| | | VGLEVIGGELAREQVEAIVNNALDKRDQKWAEVYNITKAQWWGTIHLQINTRLA |
| | | HTYKALSRQANAIKMNMEFQLANYKGNIDDKAKIKNAISETEILLNKSVEQAMKN |
| | | TEKFMIKLSNSYLTKEMIPKVQDNLKNFDLETKKTLDKFIKEKEDILGTNLSSSLRR |
| | | KVSIRLNKNIAFDINDIPFSEFDDLINQYK |
| 42 | BONT/EN | MVTINDLHYSDPIDEDNIINMRIPLYDLEVDDQFINHNVPDLKAFQVFPNVWVVPE |
| | protease and | RYTFYSTMKNLDAPANPSRSSYYDPTYLQSDAEKEVFLQQMILLFKRINSTQEGQ |
| | translocation | QFLNLLSRSIPVPYESNGDVAMGTTQVIKQMDDKGNVLKHRRAHIIIYGPGPDLM |
| | domain (1- | AKGSKALTKSRETGRGCMAEIYFSPMYHKTYSTKLTNKNSLVDKSVQEFVPDPAV |
| | 874) | TLIHELCHGLHALYGIDLGNVGSWEFNSNPNSLFSSWFSSKEAVNFEEVMTFGGE |
| | | DVKVIKSEIDKKIPGILNLIKTTVEPIINKITDPHDEMLQCLQSKYPSLKGTLGQFFF |
| | | DDTQLEKDIRDLWMVMNETMFAENLKALTRARYLVPKVENIVQVDILSPNVYTI |
| | | DKGFNHLSKGFKGQSVSQSYFRKISALARGAVVRACPNPHFSSQRGLSSCIEILED |
| | | DLFIMSSKDSFTDTDFSEPSVGPVSYKAKKGADTILDSTLSNYDFSKEINFTSTVPII |
| | | TVEDPLETDEDVPVISEDRTVYVDDYTTFHFLEAQKIGKEVVPTQTKVVFTTNME |
| | | EALFDSKKVYTVFENTASRINEAGTGIANGMMFYQWLKGIVQDFTEEATQKDTFD |
| | | KISDVTMIVPYLGNILNIGNDIRKGDFMGAVELGGVTILLEAIPELTLPVLIGLTIIED |
| | | ELEKEQVSQTVYNVLDKRDEKWEEVYGFVKQQWWWMVHTQFETRILHAYQAL |
| | | NHQVEAIKANMTYQLANYRGNQEDKELLEKAIDDTLQSLYYAVDQAMHNIKRFL |
| | | IQSSKSYLLNQMLPKTKEQLLAFDQQTLRNVNDFINKNQGVLGESLAKDLKKKVE |
| | | KRLTSLPVFNLEDLPISEFEDLIHSHEIDIQDSEVLNIGVNN |
| 43 | BONT/G | MPVNIKXFNYNDPINNDDIIMMEPFNDPGPGTYYKAFRIIDRIWIVPERFTYGFQPD |
| | protease and | QFNASTGVFSKDVYEYYDPTYLKTDAEKDKFLKTMIKLFNRINSKPSGQRLLDMI |
| | translocation | VDAIPYLGNASTPPDKFAANVANVSINKKIIQPGAEDQIKGLMTNLIIFGPGPVLSD |
| | domain (AA | NFTDSMIMNGHSPISEGFGARMMIRFCPSCLNVFNNVQENKDTSIFSRRAYFADPA |
| | 1-862) | LTLMHELIHVLHGLYGIKISNLPITPNTKEFFMQHSDPVQAEELYTFGGHDPSVISPS |
| | | TDMNIYNKALQNFQDIANRLNIVSSAQGSGIDISLYKQIYKNKYDFVEDPNGKYSV |
| | | DKDKFDKLYKALMFGFTETNLAGEYGIKTRYSYFSEYLPPIKTEKLLDNTIYTQNE |
| | | GFNIASKNLKTEFNGQNKAVNKEAYEEISLEHLVIYRIAMCKPVMYKNTGKSEQCI |
| | | IVNNEDLFFIANKDSFSKDLAKAETIAYNTQNNTIENNFSIDQLILDNDLSSGIDLPN |
| | | ENTEPFTNFDDIDIPVYIKQSALKKIFVDGDSLFEYLHAQTFPSNIENLQLTNSLNDA |
| | | LRNNNKVYTFFSTNLVEKANTVVGASLFVNWVKGVIDDFTSESTQKSTIDKVSDV |
| | | SIIIPYIGPALNVGNETAKENFKNAFEIGGAAILMEFIPELIVPIVGFFTLESYVGNKG |
| | | HIIMTISNALKKRDQKWTDMYGLIVSQWLSTVNTQFYTIKERMYNALNNQSQAIE |
| | | KIIEDQYNRYSEEDKMNINIDFNDIDFKLNQSINLAINNIDDFINQCSISYLMNRMIP |
| | | LAVKKLKDFDDNLKRDLLEYIDTNELYLLDEVNILKSKVNRHLKDSIPFDLSLYTK |
| | | DTILIQVENNYI |
| 44 | BONT/B-A2 | MPVTINNFNYNDPIDNNNIIMMEPPFARGTGRYYKAFKITDRIWIIPERYTFGYKPE |
| | chimera | DFNKSSGIFNRDVCEYYDPDYLNTNDKKNIFLQTMIKLFNRIKSKPLGEKLLEMIIN |
| | (wildtype A2 | GIPYLGDRRVPLEEFNTNIASVTVNKLISNPGEVERKKGIFANLIIFGPGPVLNENET |
| | receptor | IDIGIQNHFASREGFGGIMQMKFCPEYVSVENNVQENKGASIFNRRGYFSDPALIL |
| | binding | MHELIHVLHGLYGIKVDDLPIVPNEKKFFMQSTDAIQAEELYTFGGQDPSIITPSTD |
| | domain) | KSIYDKVLQNFRGIVDRLNKVLVCISDPNININIYKNKFKDKYKFVEDSEGKYSIDV |
| | | ESFDKLYKSLMFGFTETNIAENYKIKTRASYFSDSLPPVKIKNLLDNEIYTIEEGFNI |
| | | SDKDMEKEYRGQNKAINKQAYEEISKEHLAVYKIQMCKSVKAPGICIDVDNEDLF |
| | | FIADKNSFSDDLSKNERIEYNTQSNYIENDFPINELILDTDLISKIELPSENTESLTDF |
| | | NVDVPVYEKQPAIKKIFTDENTIFQYLYSQTFPLDIRDISLTSSFDDALLFSNKVYSF |
| | | FSMDYIKTANKVVEAGLFAGWVKQIVNDFVIEANKSNTMDKIADISLIVPYIGLAL |
| | | NVGNETAKGNFENAFEIAGASILLEFIPELLIPVVGAFLLESYIDNKNKIIKTIDNALT |
| | | KRNEKWSDMYGLIVAQWLSTVNTQFYTIKEGMYKALNYQAQALEEIIKYRYNIY |
| | | SEKEKSNINIDENDINSKLNEGINQAIDNINNFINGCSVSYLMKKMIPLAVEKLLDF |
| | | DNTLKKNLLNYIDENKLYLIGSAEYEKSKVNKYLKTIMPFDLSIYTNDTILIEMENK |
| | | YNIVNTSILSIVYKKDDLIDLSRYGAKINIGDRVYYDSIDKNQIKLINLESSTIEVILK |
| | | NAIVYNSMYENFSTSFWIKIPKYFSKINLNNEYTIINCIENNSGWKVSLNYGEIIWTL |
| | | QDNKQNIQRVVFKYSQMVNISDYINRWIFVTITNNRLTKSKIYINGRLIDQKPISNL |
| | | GNIHASNKIMFKLDGCRDPRRYIMIKYFNLFDKELNEKEIKDLYDSQSNSGILKDF |
| | | WGNYLQYDKPYYMLNLFDPNKYVDVNNIGIRGYMYLKGPRGSVVTTNIYLNSTL |
| | | YEGTKFIIKKYASGNEDNIVRNNDRVYINVVVKNKEYRLATNASQAGVEKILSAL |
| | | EIPDVGNLSQVVVMKSKDDQGIRNKCKMNLQDNNGNDIGFIGFHLYDNIAKLVAS |
| | | NWYNRQVGKASRTFGCSWEFIPVDDGWGESSL |
| 45 | BONT/C-A2 | MPITINNFNYSDPVDNKNILYLDTHLNTLANEPEKAFRITGNIWVIPDRESRNSNPN |
| | chimera | LNKPPRVTSPKSGYYDPNYLSTDSDKDTFLKEIIKLFKRINSREIGEELIYRLSTDIPF |
| | (wildtype A2 | PGNNNTPINTFDFDVDFNSVDVKTRQGNNWVKTGSINPSVIITGPRENIIDPETSTF |
| | receptor | KLTNNTFAAQEGFGALSIISISPRFMLTYSNATNDVGEGRFSKSEFCMDPILILMHE |
| | binding | LNHAMHNLYGIAIPNDQTISSVTSNIFYSQYNVKLEYAEIYAFGGPTIDLIPKSARK |
| | domain) | YFEEKALDYYRSIAKRLNSITTANPSSFNKYIGEYKQKLIRKYRFVVESSGEVTVNR |
| | | NKFVELYNELTQIFTEFNYAKIYNVQNRKIYLSNVYTPVTANILDDNVYDIQNGEN |
| | | IPKSNLNVLFMGQNLSRNPALRKVNPENMLYLFTKFCHKAIDGRSLYNKTLDCRE |
| | | LLVKNTDLPFIGDISDVKTDIFLRKDINEETEVIYYPDNVSVDQVILSKNTSEHGQL |
| | | DLLYPSIDSESEILPGENQVFYDNRTQNVDYLNSYYYLESQKLSDNVEDFTFTRSIE |
| | | EALDNSAKVYTYFPTLANKVNAGVQGGLFLMWANDVVEDFTTNILRKDTLDKIS |
| | | DVSAIIPYIGPALNISNSVRRGNFTEAFAVTGVTILLEAFPEFTIPALGAFVIYSKVQE |
| | | RNEIIKTIDNCLEQRIKRWKDSYEWMMGTWLSRIITQFNNISYQMYDSLNYQAGAI |
| | | KAKIDLEYKKYSGSDKENIKSQVENLKNSLDVKISEAMNNINKFIRECSVTYLFKN |
| | | MLPKVIDELNEFDRNTKAKLINLIDSHNIILVGEVDKLKAKVNNSFQNTIPFNIFSYT |
| | | NNSLLKDIINEYFNNINDIVNTSILSIVYKKDDLIDLSRYGAKINIGDRVYYDSIDKN |
| | | QIKLINLESSTIEVILKNAIVYNSMYENFSTSFWIKIPKYFSKINLNNEYTIINCIENNS |
| | | GWKVSLNYGEIIWTLQDNKQNIQRVVFKYSQMVNISDYINRWIFVTITNNRLTKS |
| | | KIYINGRLIDQKPISNLGNIHASNKIMFKLDGCRDPRRYIMIKYFNLFDKELNEKEIK |
| | | DLYDSQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYRLAT |
| | | NASQAGVEKILSALEIPDVGNLSQVVVMKSKDDQGIRNKCKMNLQDNNGNDIGFI |
| | | GFHLYDNIAKLVASNWYNRQVGKASRTFGCSWEFIPVDDGWGESSL |
| 46 | BONT/D-A2 | MTWPVKDFNYSDPVNDNDILYLRIPQNKLITTPVKAFMITQNIWVIPERFSSDTNPS |
| | chimera | LSKPPRPTSKYQSYYDPSYLSTDEQKDTFLKGIIKLFKRINERDIGKKLINYLVVGSP |
| | (wildtype A2 | FMGDSSTPEDTFDFTRHTTNIAVEKFENGSWKVTNIITPSVLIFGPLPNILDYTASLT |
| | receptor | LQGQQSNPSFEGFGTLSILKVAPEFLLTFSDVTSNQSSAVLGKSIFCMDPVIALMHE |
| | binding | LTHSLHQLYGINIPSDKRIRPQVSEGFFSQDGPNVQFEELYTFGGLDVEIIPQIERSQ |
| | domain) | LREKALGHYKDIAKRLNNINKTIPSSWISNIDKYKKIFSEKYNFDKDNTGNFVVNID |
| | | KFNSLYSDLTNVMSEVVYSSQYNVKNRTHYFSRHYLPVFANILDDNIYTIRDGENL |
| | | TNKGFNIENSGQNIERNPALQKLSSESVVDLFTKVCLRLTKNSRDDSTCIKVKNNR |
| | | LPYVADKDSISQEIFENKIITDETNVQNYSDNFSLDESILDGQVPINPEIVDPLLPNV |
| | | NMEPLNLPGEEIVFYDDITKYVDYLNSYYYLESQKLSNNVENITLTTSVEEALGYS |
| | | NKIYTFLPSLAEKVNKGVQAGLFLNWANEVVEDFTTNIMKKDTLDKISDVSVIIPY |
| | | IGPALNIGNSALRGNFKQAFATAGVAFLLEGFPEFTIPALGVFTFYSSIQEREKIIKTI |
| | | ENCLEQRVKRWKDSYQWMVSNWLSRITTQFNHINYQMYDSLSYQADAIKAKIDL |
| | | EYKKYSGSDKENIKSQVENLKNSLDVKISEAMNNINKFIRECSVTYLFKNMLPKVI |
| | | DELNKFDLRTKTELINLIDSHNIILVGEVDRLKAKVNESFENTMPFNIFSYTNNSLL |
| | | KDIINEYFNIVNTSILSIVYKKDDLIDLSRYGAKINIGDRVYYDSIDKNQIKLINLESS |
| | | TIEVILKNAIVYNSMYENFSTSFWIKIPKYFSKINLNNEYTIINCIENNSGWKVSLNY |
| | | GEIIWTLQDNKQNIQRVVFKYSQMVNISDYINRWIFVTITNNRLTKSKIYINGRLID |
| | | QKPISNLGNIHASNKIMFKLDGCRDPRRYIMIKYFNLFDKELNEKEIKDLYDSQSNS |
| | | GILKDFWGNYLQYDKPYYMLNLFDPNKYVDVNNIGIRGYMYLKGPRGSVVTTNI |
| | | YLNSTLYEGTKFIIKKYASGNEDNIVRNNDRVYINVVVKNKEYRLATNASQAGVE |
| | | KILSALEIPDVGNLSQVVVMKSKDDQGIRNKCKMNLQDNNGNDIGFIGFHLYDNI |
| | | AKLVASNWYNRQVGKASRTFGCSWEFIPVDDGWGESSL |
| 47 | BoNT/E-A2 | MPKINSFNYNDPVNDRTILYIKPGGCQEFYKSFNIMKNIWIIPERNVIGTTPQDFHPP |
| | chimera | TSLKNGDSSYYDPNYLQSDEEKDRFLKIVTKIFNRINNNLSGGILLEELSKANPYLG |
| | (wildtype A2 | NDNTPDNQFHIGDASAVEIKFSNGSQDILLPNVIIMGAEPDLFETNSSNISLRNNYM |
| | receptor | PSNHRFGSIAIVTFSPEYSFRFNDNCMNEFIQDPALTLMHELIHSLHGLYGAKGITT |
| | binding | KYTITQKQNPLITNIRGTNIEEFLTFGGTDLNIITSAQSNDIYTNLLADYKKIASKLS |
| | domain) | KVQVSNPLLNPYKDVFEAKYGLDKDASGIYSVNINKENDIFKKLYSFTEFDLRTKF |
| | | QVKCRQTYIGQYKYFKLSNLLNDSIYNISEGYNINNLKVNFRGQNANLNPRIITPIT |
| | | GRGLVKKIIRFCKNIVSVKGIRKSICIEINNGELFFVASENSYNDDNINTPKEIDDTV |
| | | TSNNNYENDLDQVILNFNSESAPGLSDEKLNLTIQNDAYIPKYDSNGTSDIEQHDV |
| | | NELNVFFYLDAQKVPEGENNVNLTSSIDTALLEQPKIYTFFSSEFINNVNKPVQAAL |
| | | FVSWIQQVLVDFTTEANQKSTVDKIADISIVVPYIGLALNIGNEAQKGNFKDALEL |
| | | LGAGILLEFEPELLIPTILVFTIKSFLGSSDNKNKVIKAINNALKERDEKWKEVYSFI |
| | | VSNWMTKINTQFNKRKEQMYQALQNQVNAIKTIIESKYNSYTLEEKNELTNKYDI |
| | | KQIENELNQKVSIAMNNIDRFLTESSISYLMKIINEVKINKLREYDENVKTYLLNYII |
| | | QHGSILGESQQELNSMVTDTLNNSIPFKLSSYTDDKILISYFNKFFIVNTSILSIVYKK |
| | | DDLIDLSRYGAKINIGDRVYYDSIDKNQIKLINLESSTIEVILKNAIVYNSMYENFST |
| | | SFWIKIPKYFSKINLNNEYTIINCIENNSGWKVSLNYGEIIWTLQDNKQNIQRVVFK |
| | | YSQMVNISDYINRWIFVTITNNRLTKSKIYINGRLIDQKPISNLGNIHASNKIMFKLD |
| | | GCRDPRRYIMIKYFNLFDKELNEKEIKDLYDSQSNSGILKDFWGNYLQYDKPYYM |
| | | LNLFDPNKYVDVNNIGIRGYMYLKGPRGSVVTTNIYLNSTLYEGTKFIIKKYASGN |
| | | EDNIVRNNDRVYINVVVKNKEYRLATNASQAGVEKILSALEIPDVGNLSQVVVM |
| | | KSKDDQGIRNKCKMNLQDNNGNDIGFIGFHLYDNIAKLVASNWYNRQVGKASRT |
| | | FGCSWEFIPVDDGWGESSL |
| 48 | BONT/F-A2 | MPVVINSFNYNDPVNDDTILYMQIPYEEKSKKYYKAFEIMRNVWIIPERNTIGTDP |
| | chimera | SDFDPPASLENGSSAYYDPNYLTTDAEKDRYLKTTIKLFKRINSNPAGEVLLQEISY |
| | (wildtype A2 | AKPYLGNEHTPINEFHPVTRTTSVNIKSSTNVKSSIILNLLVLGAGPDIFENSSYPVR |
| | receptor | KLMDSGGVYDPSNDGFGSINIVTFSPEYEYTFNDISGGYNSSTESFIADPAISLAHEL |
| | binding | IHALHGLYGARGVTYKETIKVKQAPLMIAEKPIRLEEFLTFGGQDLNIITSAMKEKI |
| | domain) | YNNLLANYEKIATRLSRVNSAPPEYDINEYKDYFQWKYGLDKNADGSYTVNENK |
| | | FNEIYKKLYSFTEIDLANKFKVKCRNTYFIKYGFLKVPNLLDDDIYTVSEGFNIGNL |
| | | AVNNRGQNIKLNPKIIDSIPDKGLVEKIVKFCKSVIPRKGTKAPPRLCIRVNNRELFF |
| | | VASESSYNENDINTPKEIDDTTNLNNNYRNNLDEVILDYNSETIPQISNQTLNTLVQ |
| | | DDSYVPRYDSNGTSEIEEHNVVDLNVFFYLHAQKVPEGETNISLTSSIDTALSEESQ |
| | | VYTFFSSEFINTINKPVHAALFISWINQVIRDFTTEATQKSTFDKIADISLVVPYVGL |
| | | ALNIGNEVQKENFKEAFELLGAGILLEFVPELLIPTILVFTIKSFIGSSENKNKIIKAIN |
| | | NSLMERETKWKEIYSWIVSNWLTRINTQFNKRKEQMYQALQNQVDAIKTVIEYK |
| | | YNNYTSDERNRLESEYNINNIREELNKKVSLAMENIERFITESSIFYLMKLINEAKV |
| | | SKLREYDEGVKEYLLDYISEHRSILGNSVQELNDLVTSTLNNSIPFELSSYTNDKILI |
| | | LYFNKLYIVNTSILSIVYKKDDLIDLSRYGAKINIGDRVYYDSIDKNQIKLINLESSTI |
| | | EVILKNAIVYNSMYENFSTSFWIKIPKYFSKINLNNEYTIINCIENNSGWKVSLNYG |
| | | EIIWTLQDNKQNIQRVVFKYSQMVNISDYINRWIFVTITNNRLTKSKIYINGRLIDQ |
| | | KPISNLGNIHASNKIMFKLDGCRDPRRYIMIKYFNLFDKELNEKEIKDLYDSQSNSG |
| | | ILKDFWGNYLQYDKPYYMLNLFDPNKYVDVNNIGIRGYMYLKGPRGSVVTTNIY |
| | | LNSTLYEGTKFIIKKYASGNEDNIVRNNDRVYINVVVKNKEYRLATNASQAGVEK |
| | | ILSALEIPDVGNLSQVVVMKSKDDQGIRNKCKMNLQDNNGNDIGFIGFHLYDNIA |
| | | KLVASNWYNRQVGKASRTFGCSWEFIPVDDGWGESSL |
| 49 | BoNT/G-A2 | MPVNIKXFNYNDPINNDDIIMMEPFNDPGPGTYYKAFRIIDRIWIVPERFTYGFQPD |
| | chimera | QFNASTGVFSKDVYEYYDPTYLKTDAEKDKFLKTMIKLFNRINSKPSGQRLLDMI |
| | (wildtype A2 | VDAIPYLGNASTPPDKFAANVANVSINKKIIQPGAEDQIKGLMTNLIIFGPGPVLSD |
| | receptor | NFTDSMIMNGHSPISEGFGARMMIRFCPSCLNVENNVQENKDTSIFSRRAYFADPA |
| | binding | LTLMHELIHVLHGLYGIKISNLPITPNTKEFFMQHSDPVQAEELYTFGGHDPSVISPS |
| | domain) | TDMNIYNKALQNFQDIANRLNIVSSAQGSGIDISLYKQIYKNKYDFVEDPNGKYSV |
| | | DKDKFDKLYKALMFGFTETNLAGEYGIKTRYSYFSEYLPPIKTEKLLDNTIYTQNE |
| | | GFNIASKNLKTEFNGQNKAVNKEAYEEISLEHLVIYRIAMCKPVMYKNTGKSEQCI |
| | | IVNNEDLFFIANKDSFSKDLAKAETIAYNTQNNTIENNFSIDQLILDNDLSSGIDLPN |
| | | ENTEPFTNFDDIDIPVYIKQSALKKIFVDGDSLFEYLHAQTFPSNIENLQLTNSLNDA |
| | | LRNNNKVYTFFSTNLVEKANTVVGASLFVNWVKGVIDDFTSESTQKSTIDKVSDV |
| | | SIIIPYIGPALNVGNETAKENFKNAFEIGGAAILMEFIPELIVPIVGFFTLESYVGNKG |
| | | HIIMTISNALKKRDQKWTDMYGLIVSQWLSTVNTQFYTIKERMYNALNNQSQAIE |
| | | KIIEDQYNRYSEEDKMNINIDENDIDFKLNQSINLAINNIDDFINQCSISYLMNRMIP |
| | | LAVKKLKDFDDNLKRDLLEYIDTNELYLLDEVNILKSKVNRHLKDSIPFDLSLYTK |
| | | DTILIQVENNYIIVNTSILSIVYKKDDLIDLSRYGAKINIGDRVYYDSIDKNQIKLINL |
| | | ESSTIEVILKNAIVYNSMYENFSTSFWIKIPKYFSKINLNNEYTIINCIENNSGWKVSL |
| | | NYGEIIWTLQDNKQNIQRVVFKYSQMVNISDYINRWIFVTITNNRLTKSKIYINGRL |
| | | IDQKPISNLGNIHASNKIMFKLDGCRDPRRYIMIKYFNLFDKELNEKEIKDLYDSQS |
| | | NSGILKDFWGNYLQYDKPYYMLNLFDPNKYVDVNNIGIRGYMYLKGPRGSVVTT |
| | | NIYLNSTLYEGTKFIIKKYASGNEDNIVRNNDRVYINVVVKNKEYRLATNASQAG |
| | | VEKILSALEIPDVGNLSQVVVMKSKDDQGIRNKCKMNLQDNNGNDIGFIGFHLYD |
| | | NIAKLVASNWYNRQVGKASRTFGCSWEFIPVDDGWGESSL |
| 50 | BONT/H-A2 | MPVVINSFNYDDPVNDNTIIYIRPPYYETSNTYFKAFQIMDNVWIIPERYRLGIDPSL |
| | chimera | FNPPVSLKAGSDGYFDPNYLSTNTEKNKYLQIMIKLFKRINSKPAGQILLEEIKNAIP |
| | (wildtype A2 | YLGNSYTQEEQFTTNNRTVSFNVKLANGNIVQQMANLIIWGPGPDLTTNKTGGIIY |
| | receptor | SPYQSMEATPYKDGFGSIMTVEFSPEYATAFNDISIASHSPSLFIKDPALILMHELIH |
| | binding | VLHGLYGTYITEYKITPNVVQSYMKVTKPITSAEFLTFGGRDRNIVPQSIQSQLYNK |
| | domain) | VLSDYKRIASRLNKVNTATALINIDEFKNLYEWKYQFAKDSNGVYSVDLNKFEQL |
| | | YKKIYSFTEFNLAYEFKIKTRLGYLAENFGPFYLPNLLDDSIYTEVDGFNIGALSIN |
| | | YQGQNIGSDINSIKKLQGQGVVSRVVRLCSNSNTKNSLCITVNNRDLFFIASQESY |
| | | GENTINTYKEIDDTTTLDPSFEDILDKVILNFNEQVIPQMPNRNVSTDIQKDNYIPK |
| | | YDYNRTDIIDSYEVGRNYNTFFYLNAQKFSPNESNITLTSSFDTGLLEGSKVYTFFS |
| | | SDFINNINKPVQALLFIEWVKQVIRDFTTEATKTSTVDKLKDISLVVPYIGLALNIG |
| | | DEIYKQHFAEAVELVGAGLLLEFSPEFLIPTLLIFTIKGYLTGSIRDKDKIIKTLDNAL |
| | | NVRDQKWKELYRWVVSKWLTTINTQFNKRKEQMYKALKNQATAIKKIIENKYN |
| | | NYTTDEKSKIDSSYNINEIERTLNEKINLAMKNIEQFITESSIAYLINIINNETIQKLKS |
| | | YDDLVRRYLLGYIRNHSSILGNSVEELNSKVNNHLDNGIPFELSSYTNDSLLIRYFN |
| | | KNYIVNTSILSIVYKKDDLIDLSRYGAKINIGDRVYYDSIDKNQIKLINLESSTIEVIL |
| | | KNAIVYNSMYENFSTSFWIKIPKYFSKINLNNEYTIINCIENNSGWKVSLNYGEIIW |
| | | TLQDNKQNIQRVVFKYSQMVNISDYINRWIFVTITNNRLTKSKIYINGRLIDQKPIS |
| | | NLGNIHASNKIMFKLDGCRDPRRYIMIKYFNLFDKELNEKEIKDLYDSQSNSGILK |
| | | DFWGNYLQYDKPYYMLNLFDPNKYVDVNNIGIRGYMYLKGPRGSVVTTNIYLNS |
| | | TLYEGTKFIIKKYASGNEDNIVRNNDRVYINVVVKNKEYRLATNASQAGVEKILS |
| | | ALEIPDVGNLSQVVVMKSKDDQGIRNKCKMNLQDNNGNDIGFIGFHLYDNIAKL |
| | | VASNWYNRQVGKASRTFGCSWEFIPVDDGWGESSL |
| 51 | BONT/X-A2 | MKLEINKFNYNDPIDGINVITMRPPRHSDKINKGKGPFKAFQVIKNIWIVPERYNFT |
| | chimera | NNTNDLNIPSEPIMEADAIYNPNYLNTPSEKDEFLQGVIKVLERIKSKPEGEKLLELI |
| | (wildtype A2 | SSSIPLPLVSNGALTLSDNETIAYQENNNIVSNLQANLVIYGPGPDIANNATYGLYS |
| | receptor | TPISNGEGTLSEVSFSPFYLKPFDESYGNYRSLVNIVNKFVKREFAPDPASTLMHEL |
| | binding | VHVTHNLYGISNRNFYYNFDTGKIETSRQQNSLIFEELLTFGGIDSKAISSLIIKKIIE |
| | domain) | TAKNNYTTLISERLNTVTVENDLLKYIKNKIPVQGRLGNFKLDTAEFEKKLNTILF |
| | | VLNESNLAQRFSILVRKHYLKERPIDPIYVNILDDNSYSTLEGFNISSQGSNDFQGQ |
| | | LLESSYFEKIESNALRAFIKICPRNGLLYNAIYRNSKNYLNNIDLEDKKTTSKTNVS |
| | | YPCSLLNGCIEVENKDLFLISNKDSLNDINLSEEKIKPETTVFFKDKLPPQDITLSNY |
| | | DFTEANSIPSISQQNILERNEELYEPIRNSLFEIKTIYVDKLTTFHFLEAQNIDESIDSS |
| | | KIRVELTDSVDEALSNPNKVYSPFKNMSNTINSIETGITSTYIFYQWLRSIVKDESDE |
| | | TGKIDVIDKSSDTLAIVPYIGPLLNIGNDIRHGDFVGAIELAGITALLEYVPEFTIPIL |
| | | VGLEVIGGELAREQVEAIVNNALDKRDQKWAEVYNITKAQWWGTIHLQINTRLA |
| | | HTYKALSRQANAIKMNMEFQLANYKGNIDDKAKIKNAISETEILLNKSVEQAMKN |
| | | TEKFMIKLSNSYLTKEMIPKVQDNLKNFDLETKKTLDKFIKEKEDILGTNLSSSLRR |
| | | KVSIRLNKNIAFDINDIPFSEFDDLINQYKIVNTSILSIVYKKDDLIDLSRYGAKINIG |
| | | DRVYYDSIDKNQIKLINLESSTIEVILKNAIVYNSMYENFSTSFWIKIPKYFSKINLN |
| | | NEYTIINCIENNSGWKVSLNYGEIIWTLQDNKQNIQRVVFKYSQMVNISDYINRWI |
| | | FVTITNNRLTKSKIYINGRLIDQKPISNLGNIHASNKIMFKLDGCRDPRRYIMIKYFN |
| | | LFDKELNEKEIKDLYDSQSNSGILKDFWGNYLQYDKPYYMLNLFDPNKYVDVNN |
| | | IGIRGYMYLKGPRGSVVTTNIYLNSTLYEGTKFIIKKYASGNEDNIVRNNDRVYIN |
| | | VVVKNKEYRLATNASQAGVEKILSALEIPDVGNLSQVVVMKSKDDQGIRNKCKM |
| | | NLQDNNGNDIGFIGFHLYDNIAKLVASNWYNRQVGKASRTFGCSWEFIPVDDGW |
| | | GESSL |
| 52 | BONT/EN-A2 | MVTINDLHYSDPIDEDNIINMRIPLYDLEVDDQFINHNVPDLKAFQVFPNVWVVPE |
| | chimera | RYTFYSTMKNLDAPANPSRSSYYDPTYLQSDAEKEVFLQQMILLFKRINSTQEGQ |
| | (wildtype A2 | QFLNLLSRSIPVPYESNGDVAMGTTQVIKQMDDKGNVLKHRRAHIIIYGPGPDLM |
| | receptor | AKGSKALTKSRETGRGCMAEIYFSPMYHKTYSTKLTNKNSLVDKSVQEFVPDPAV |
| | binding | TLIHELCHGLHALYGIDLGNVGSWEFNSNPNSLFSSWFSSKEAVNFEEVMTFGGE |
| | domain) | DVKVIKSEIDKKIPGILNLIKTTVEPIINKITDPHDEMLQCLQSKYPSLKGTLGQFFF |
| | | DDTQLEKDIRDLWMVMNETMFAENLKALTRARYLVPKVENIVQVDILSPNVYTI |
| | | DKGFNHLSKGFKGQSVSQSYFRKISALARGAVVRACPNPHFSSQRGLSSCIEILED |
| | | DLFIMSSKDSFTDTDFSEPSVGPVSYKAKKGADTILDSTLSNYDFSKEINFTSTVPII |
| | | TVEDPLETDEDVPVISEDRTVYVDDYTTFHFLEAQKIGKEVVPTQTKVVFTTNME |
| | | EALFDSKKVYTVFENTASRINEAGTGIANGMMFYQWLKGIVQDFTEEATQKDTFD |
| | | KISDVTMIVPYLGNILNIGNDIRKGDFMGAVELGGVTILLEAIPELTLPVLIGLTIIED |
| | | ELEKEQVSQTVYNVLDKRDEKWEEVYGFVKQQWWWMVHTQFETRILHAYQAL |
| | | NHQVEAIKANMTYQLANYRGNQEDKELLEKAIDDTLQSLYYAVDQAMHNIKRFL |
| | | IQSSKSYLLNQMLPKTKEQLLAFDQQTLRNVNDFINKNQGVLGESLAKDLKKKVE |
| | | KRLTSLPVFNLEDLPISEFEDLIHSHEIDIQDSEVLNIGVNNIVNTSILSIVYKKDDLID |
| | | LSRYGAKINIGDRVYYDSIDKNQIKLINLESSTIEVILKNAIVYNSMYENFSTSFWIK |
| | | IPKYFSKINLNNEYTIINCIENNSGWKVSLNYGEIIWTLQDNKQNIQRVVFKYSQM |
| | | VNISDYINRWIFVTITNNRLTKSKIYINGRLIDQKPISNLGNIHASNKIMFKLDGCRD |
| | | PRRYIMIKYFNLFDKELNEKEIKDLYDSQSNSGILKDFWGNYLQYDKPYYMLNLF |
| | | DPNKYVDVNNIGIRGYMYLKGPRGSVVTTNIYLNSTLYEGTKFIIKKYASGNEDNI |
| | | VRNNDRVYINVVVKNKEYRLATNASQAGVEKILSALEIPDVGNLSQVVVMKSKD |
| | | DQGIRNKCKMNLQDNNGNDIGFIGFHLYDNIAKLVASNWYNRQVGKASRTFGCS |
| | | WEFIPVDDGWGESSL |
| 53 | Thrombin | LVPRGS |
| | cleavage | |
| | sequence | |
| 54 | TEV cleavage | ENLYFQG |
| | sequence | |
| 55 | PreScission | LEVLFQGP |
| | cleavage | |
| | sequence 1 | |
| 56 | Factor Xa | IEGR |
| | cleavage | |
| | sequence 1 | |
| 57 | Factor Xa | IDGR |
| | cleavage | |
| | sequence 2 | |
| 58 | Enterokinase | DDDDK |
| 59 | SUMO | AHREQIGG |
| | protease | |
| 60 | BONT/A2- | MPFVNKQFNYKDPVNGVDIAYIKIPNAGQMQPVKAFKIHNKIWVIPERDTFTNPEE |
| | R1064Y | GDLNPPPEAKQVPVSYYDSTYLSTDNEKDNYLKGVTKLFERIYSTDLGRMLLTSIV |
| | | RGIPFWGGSTIDTELKVIDTNCINVIQPDGSYRSEELNLVIIGPSADIIQFECKSFGHD |
| | | VLNLTRNGYGSTQYIRFSPDFTFGFEESLEVDTNPLLGAGKFATDPAVTLAHELIH |
| | | AEHRLYGIAINPNRVFKVNTNAYYEMSGLEVSFEELRTFGGHDAKFIDSLQENEFR |
| | | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSLDEGYNKALN |
| | | DLCIKVNNWDLFFSPSEDNFTNDLDKVEEITADTNIEAAEENISLDLIQQYYLTFDF |
| | | DNEPENISIENLSSDIIGQLEPMPNIERFPNGKKYELDKYTMFHYLRAQEFEHGDSRI |
| | | ILTNSAEEALLKPNVAYTFFSSKYVKKINKAVEAFMFLNWAEELVYDFTDETNEV |
| | | TTMDKIADITIIVPYIGPALNIGNMLSKGEFVEAIIFTGVVAMLEFIPEYALPVFGTF |
| | | AIVSYIANKVLTVQTINNALSKRNEKWDEVYKYTVTNWLAKVNTQIDLIREKMK |
| | | KALENQAEATKAIINYQYNQYTEEEKNNINFNIDDLSSKLNESINSAMININKFLDQ |
| | | CSVSYLMNSMIPYAVKRLKDFDASVRDVLLKYIYDNRGTLVLQVDRLKDEVNNT |
| | | LSADIPFQLSKYVDNKKLLSTFTEYIKNIVNTSILSIVYKKDDLIDLSRYGAKINIGD |
| | | RVYYDSIDKNQIKLINLESSTIEVILKNAIVYNSMYENFSTSFWIKIPKYFSKINLNN |
| | | EYTIINCIENNSGWKVSLNYGEIIWTLQDNKQNIQRVVFKYSQMVNISDYINRWIF |
| | | VTITNNRLTKSKIYINGRLIDQKPISNLGNIHASNKIMFKLDGCRDPYRYIMIKYFNL |
| | | FDKELNEKEIKDLYDSQSNSGILKDFWGNYLQYDKPYYMLNLFDPNKYVDVNNI |
| | | GIRGYMYLKGPRGSVVTTNIYLNSTLYEGTKFIIKKYASGNEDNIVRNNDRVYINV |
| | | VVKNKEYRLATNASQAGVEKILSALEIPDVGNLSQVVVMKSKDDQGIRNKCKMN |
| | | LQDNNGNDIGFIGFHLYDNIAKLVASNWYNRQVGKASRTFGCSWEFIPVDDGWG |
| | | ESSL |
| 61 | BONT/A2- | MPFVNKQFNYKDPVNGVDIAYIKIPNAGQMQPVKAFKIHNKIWVIPERDTFTNPEE |
| | R1064A | GDLNPPPEAKQVPVSYYDSTYLSTDNEKDNYLKGVTKLFERIYSTDLGRMLLTSIV |
| | | RGIPFWGGSTIDTELKVIDTNCINVIQPDGSYRSEELNLVIIGPSADIIQFECKSFGHD |
| | | VLNLTRNGYGSTQYIRFSPDFTFGFEESLEVDTNPLLGAGKFATDPAVTLAHELIH |
| | | AEHRLYGIAINPNRVFKVNTNAYYEMSGLEVSFEELRTFGGHDAKFIDSLQENEFR |
| | | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSLDEGYNKALN |
| | | DLCIKVNNWDLFFSPSEDNFTNDLDKVEEITADTNIEAAEENISLDLIQQYYLTFDF |
| | | DNEPENISIENLSSDIIGQLEPMPNIERFPNGKKYELDKYTMFHYLRAQEFEHGDSRI |
| | | ILTNSAEEALLKPNVAYTFFSSKYVKKINKAVEAFMFLNWAEELVYDFTDETNEV |
| | | TTMDKIADITIIVPYIGPALNIGNMLSKGEFVEAIIFTGVVAMLEFIPEYALPVFGTF |
| | | AIVSYIANKVLTVQTINNALSKRNEKWDEVYKYTVTNWLAKVNTQIDLIREKMK |
| | | KALENQAEATKAIINYQYNQYTEEEKNNINFNIDDLSSKLNESINSAMININKFLDQ |
| | | CSVSYLMNSMIPYAVKRLKDFDASVRDVLLKYIYDNRGTLVLQVDRLKDEVNNT |
| | | LSADIPFQLSKYVDNKKLLSTFTEYIKNIVNTSILSIVYKKDDLIDLSRYGAKINIGD |
| | | RVYYDSIDKNQIKLINLESSTIEVILKNAIVYNSMYENFSTSFWIKIPKYFSKINLNN |
| | | EYTIINCIENNSGWKVSLNYGEIIWTLQDNKQNIQRVVFKYSQMVNISDYINRWIF |
| | | VTITNNRLTKSKIYINGRLIDQKPISNLGNIHASNKIMFKLDGCRDPARYIMIKYFNL |
| | | FDKELNEKEIKDLYDSQSNSGILKDFWGNYLQYDKPYYMLNLFDPNKYVDVNNI |
| | | GIRGYMYLKGPRGSVVTTNIYLNSTLYEGTKFIIKKYASGNEDNIVRNNDRVYINV |
| | | VVKNKEYRLATNASQAGVEKILSALEIPDVGNLSQVVVMKSKDDQGIRNKCKMN |
| | | LQDNNGNDIGFIGFHLYDNIAKLVASNWYNRQVGKASRTFGCSWEFIPVDDGWG |
| | | ESSL |
| 62 | HC- | DLIDLSRYGAKINIGDRVYYDSIDKNQIKLINLESSTIEVILKNAIVYNSMYENFSTS |
| | BONT/A2- | FWIKIPKYFSKINLNNEYTIINCIENNSGWKVSLNYGEIIWTLQDNKQNIQRVVFKY |
| | R1064Y | SQMVNISDYINRWIFVTITNNRLTKSKIYINGRLIDQKPISNLGNIHASNKIMFKLDG |
| | | CRDPYRYIMIKYFNLFDKELNEKEIKDLYDSQSNSGILKDFWGNYLQYDKPYYML |
| | | NLFDPNKYVDVNNIGIRGYMYLKGPRGSVVTTNIYLNSTLYEGTKFIIKKYASGNE |
| | | DNIVRNNDRVYINVVVKNKEYRLATNASQAGVEKILSALEIPDVGNLSQVVVMKS |
| | | KDDQGIRNKCKMNLQDNNGNDIGFIGFHLYDNIAKLVASNWYNRQVGKASRTFG |
| | | CSWEFIPVDDGWGESSL |
| 63 | HC- | DLIDLSRYGAKINIGDRVYYDSIDKNQIKLINLESSTIEVILKNAIVYNSMYENESTS |
| | BONT/A2- | FWIKIPKYFSKINLNNEYTIINCIENNSGWKVSLNYGEIIWTLQDNKQNIQRVVFKY |
| | R1064A | SQMVNISDYINRWIFVTITNNRLTKSKIYINGRLIDQKPISNLGNIHASNKIMFKLDG |
| | | CRDPARYIMIKYFNLFDKELNEKEIKDLYDSQSNSGILKDFWGNYLQYDKPYYML |
| | | NLFDPNKYVDVNNIGIRGYMYLKGPRGSVVTTNIYLNSTLYEGTKFIIKKYASGNE |
| | | DNIVRNNDRVYINVVVKNKEYRLATNASQAGVEKILSALEIPDVGNLSQVVVMKS |
| | | KDDQGIRNKCKMNLQDNNGNDIGFIGFHLYDNIAKLVASNWYNRQVGKASRTFG |
| | | CSWEFIPVDDGWGESSL |
| 64 | BONT/A2 | MPFVNKQFNYKDPVNGVDIAYIKIPNAGQMQPVKAFKIHNKIWVIPERDTFTNPEE |
| | wildtype with | GDLNPPPEAKQVPVSYYDSTYLSTDNEKDNYLKGVTKLFERIYSTDLGRMLLTSIV |
| | a thrombin | RGIPFWGGSTIDTELKVIDTNCINVIQPDGSYRSEELNLVIIGPSADIIQFECKSFGHD |
| | cleavage site | VLNLTRNGYGSTQYIRFSPDFTFGFEESLEVDTNPLLGAGKFATDPAVTLAHELIH |
| | between LC | AEHRLYGIAINPNRVFKVNTNAYYEMSGLEVSFEELRTFGGHDAKFIDSLQENEFR |
| | and HC | LYYYNKFKDVASTLNKAKSIIGTTASLQYMKNVFKEKYLLSEDTSGKFSVDKLKF |
| | | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSASLVPRGSGG |
| | | AAALDEGYNKALNDLCIKVNNWDLFFSPSEDNFTNDLDKVEEITADTNIEAAEENI |
| | | SLDLIQQYYLTFDFDNEPENISIENLSSDIIGQLEPMPNIERFPNGKKYELDKYTMFH |
| | | YLRAQEFEHGDSRIILTNSAEEALLKPNVAYTFFSSKYVKKINKAVEAFMFLNWAE |
| | | ELVYDFTDETNEVTTMDKIADITIIVPYIGPALNIGNMLSKGEFVEAIIFTGVVAML |
| | | EFIPEYALPVFGTFAIVSYIANKVLTVQTINNALSKRNEKWDEVYKYTVTNWLAK |
| | | VNTQIDLIREKMKKALENQAEATKAIINYQYNQYTEEEKNNINFNIDDLSSKLNESI |
| | | NSAMININKFLDQCSVSYLMNSMIPYAVKRLKDFDASVRDVLLKYIYDNRGTLVL |
| | | QVDRLKDEVNNTLSADIPFQLSKYVDNKKLLSTFTEYIKNIVNTSILSIVYKKDDLI |
| | | DLSRYGAKINIGDRVYYDSIDKNQIKLINLESSTIEVILKNAIVYNSMYENFSTSFWI |
| | | KIPKYFSKINLNNEYTIINCIENNSGWKVSLNYGEIIWTLQDNKQNIQRVVFKYSQ |
| | | MVNISDYINRWIFVTITNNRLTKSKIYINGRLIDQKPISNLGNIHASNKIMFKLDGCR |
| | | DPRRYIMIKYFNLFDKELNEKEIKDLYDSQSNSGILKDFWGNYLQYDKPYYMLNL |
| | | FDPNKYVDVNNIGIRGYMYLKGPRGSVVTTNIYLNSTLYEGTKFIIKKYASGNEDN |
| | | IVRNNDRVYINVVVKNKEYRLATNASQAGVEKILSALEIPDVGNLSQVVVMKSKD |
| | | DQGIRNKCKMNLQDNNGNDIGFIGFHLYDNIAKLVASNWYNRQVGKASRTFGCS |
| | | WEFIPVDDGWGESSL |
| 65 | BONT/A2- | MPFVNKQFNYKDPVNGVDIAYIKIPNAGQMQPVKAFKIHNKIWVIPERDTFTNPEE |
| | R1064H- | GDLNPPPEAKQVPVSYYDSTYLSTDNEKDNYLKGVTKLFERIYSTDLGRMLLTSIV |
| | S1294R- | RGIPFWGGSTIDTELKVIDTNCINVIQPDGSYRSEELNLVIIGPSADIIQFECKSFGHD |
| | S1295P + | VLNLTRNGYGSTQYIRFSPDFTFGFEESLEVDTNPLLGAGKFATDPAVTLAHELIH |
| | Thrombin | AEHRLYGIAINPNRVFKVNTNAYYEMSGLEVSFEELRTFGGHDAKFIDSLQENEFR |
| | Linker (A2- | LYYYNKFKDVASTLNKAKSIIGTTASLQYMKNVFKEKYLLSEDTSGKFSVDKLKF |
| | HRP) | DKLYKMLTEIYTEDNFVNFFKVINRKTYLNFDKAVFRINIVPDENYTIKDGFNLKG |
| | | ANLSTNFNGQNTEINSRNFTRLKNFTGLFEFYKLLCVRGIIPFKTKSASLVPRGSGG |
| | | AAALDEGYNKALNDLCIKVNNWDLFFSPSEDNFTNDLDKVEEITADTNIEAAEENI |
| | | SLDLIQQYYLTFDFDNEPENISIENLSSDIIGQLEPMPNIERFPNGKKYELDKYTMFH |
| | | YLRAQEFEHGDSRIILTNSAEEALLKPNVAYTFFSSKYVKKINKAVEAFMFLNWAE |
| | | ELVYDFTDETNEVTTMDKIADITIIVPYIGPALNIGNMLSKGEFVEAIIFTGVVAML |
| | | EFIPEYALPVFGTFAIVSYIANKVLTVQTINNALSKRNEKWDEVYKYTVTNWLAK |
| | | VNTQIDLIREKMKKALENQAEATKAIINYQYNQYTEEEKNNINFNIDDLSSKLNESI |
| | | NSAMININKFLDQCSVSYLMNSMIPYAVKRLKDFDASVRDVLLKYIYDNRGTLVL |
| | | QVDRLKDEVNNTLSADIPFQLSKYVDNKKLLSTFTEYIKNIVNTSILSIVYKKDDLI |
| | | DLSRYGAKINIGDRVYYDSIDKNQIKLINLESSTIEVILKNAIVYNSMYENFSTSFWI |
| | | KIPKYFSKINLNNEYTIINCIENNSGWKVSLNYGEIIWTLQDNKQNIQRVVFKYSQ |
| | | MVNISDYINRWIFVTITNNRLTKSKIYINGRLIDQKPISNLGNIHASNKIMFKLDGCR |
| | | DPHRYIMIKYFNLFDKELNEKEIKDLYDSQSNSGILKDFWGNYLQYDKPYYMLNL |
| | | FDPNKYVDVNNIGIRGYMYLKGPRGSVVTTNIYLNSTLYEGTKFIIKKYASGNEDN |
| | | IVRNNDRVYINVVVKNKEYRLATNASQAGVEKILSALEIPDVGNLSQVVVMKSKD |
| | | DQGIRNKCKMNLQDNNGNDIGFIGFHLYDNIAKLVASNWYNRQVGKASRTFGCS |
| | | WEFIPVDDGWGERPL |

### EXAMPLES

### Example 1

Botulinum neurotoxins (BoNTs) are a family of related bacterial toxins. Each type also contain multiple subtypes (BoNT/A1, A2, etc.). BoNTs act by blocking neurotransmitter release from neurons. In recent years, BoNTs have been widely used to treat a growing list of medical conditions as well as for cosmetic purposes. BoNT/A1 (Botox/Dysport) and BoNT/B1 (Myobloc) are the only two BoNTs that are FDA-approved for medical and cosmetic uses.

One of the major areas that need further improvement for current BoNTs is to reduce chances of inducing immune response against the BoNT and resulting generation of neutralizing antibodies in patients, which renders future BoNT treatment ineffective. The chance of inducing neutralizing antibodies is largely related to the total dose of toxins: the higher the toxin dose injected, the higher chance for inducing antibodies. One way to reduce the chance of inducing neutralizing antibodies, is to enhance the overall efficacy of toxins, so less toxin would be injected to achieve the same level of local paralysis. One way to enhance the efficacy of toxins locally is to improve BoNT targeting to neuronal receptors as (1) efficient entry into neurons will reduce the chance of immune responses since internalized toxins would not be recognized by immune systems after cell entry, and (2) less BoNT will need to be administered to the patient.

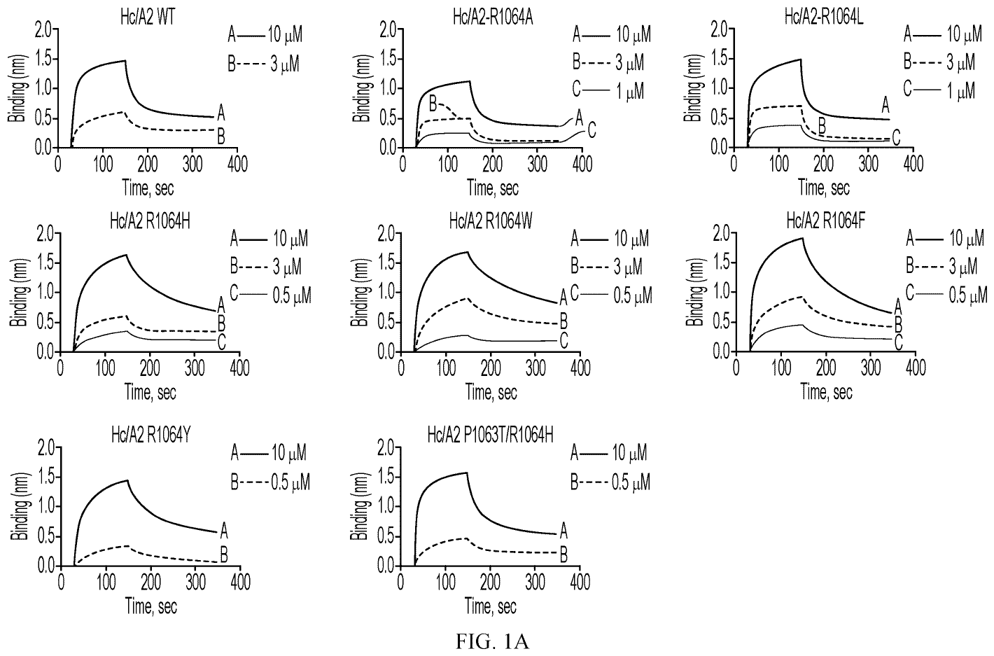

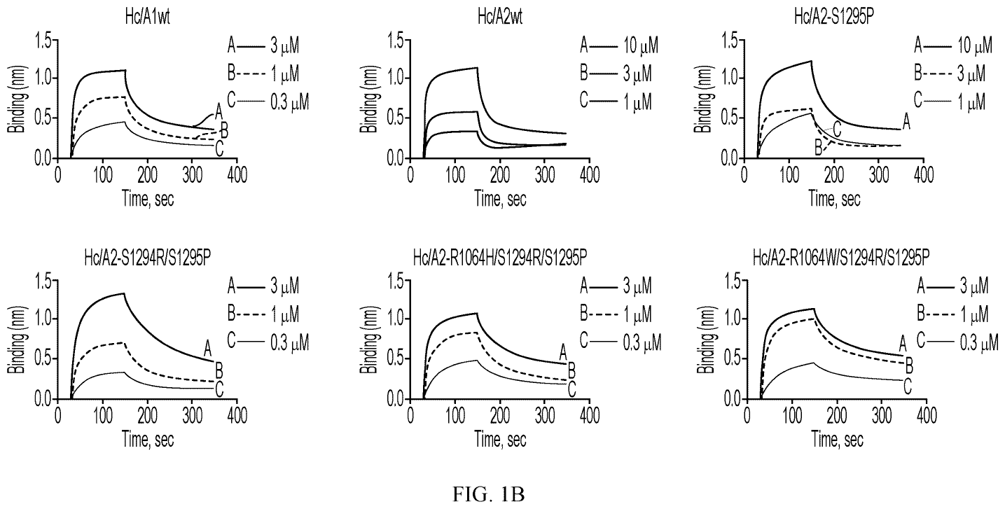

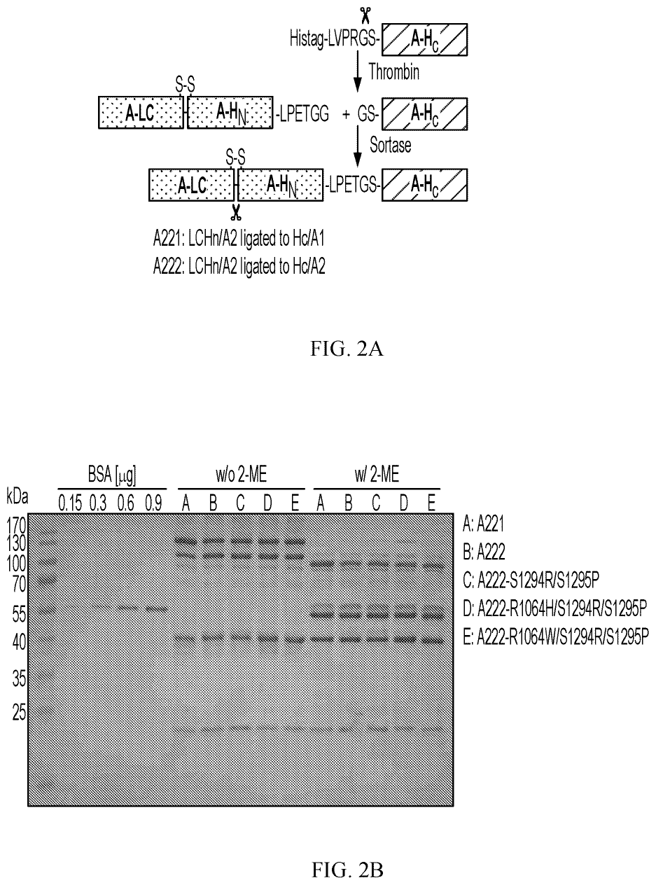

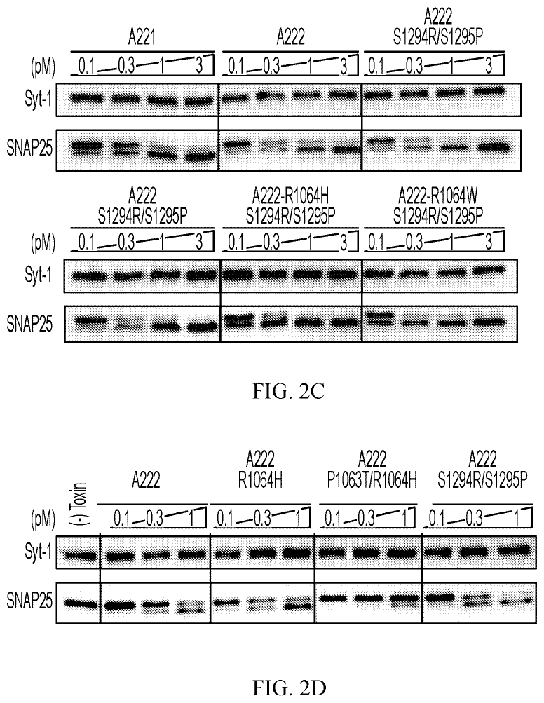

BoNT/A2 has been reported to have naturally higher potency than BoNT/A1 and faster onset time. There are about 10% sequence differences between BoNT/A2 and BoNT/A. Here consensus mutagenesis was performed (by comparing the sequences of different BoNT/A variants) to introduce mutations into BoNT/A2, replacing the native residue in BoNT/A2-Hc (receptor binding domain) with the ones found in BoNT/A1-Hc. The resulting BoNT/A2-mutant (R1064W/F/H, S1294R, S1295P) showed higher activity than native BoNT/A2 in binding to protein receptors (FIG. 1A-1B and Table 2) and high activity in neuronal assays (FIG. 2B-2D).

Binding of BoNT/A2-Hc mutants was measured using a Biolayer Interferometry (BLI) assay. Results show that the mutants tested all increase binding affinity of the BoNT/A2 for the glycosylated synaptic vesicle glycoprotein 2C (SV2C) used in the assay (FIG. 1A-1B). Additionally, full length BoNTs including the mutated BoNT/A2-Hc domains and the catalytic and transmembrane domain (LCHn) were produced using a sortase ligation (FIG. 2A-2B) and shown to cleave neuron SNAP-25 cell surface proteins better than the wildtype BoNT/A2 protein (FIG. 2C-2D). Specifically, a triple mutant comprising either R1064H or R1064 W and S1294R/S1295P increased cleavage efficiency by 3-fold.

Overall these results show that mutations R1064W/F/H/L and S1294R/S1295P increase BoNT binding to target receptors and therefore the modified BoNT/A2 proteins disclosed herein can be administered at lower doses, but have a similar effect to wildtype BoNT/A2. This is advantageous because administering BoNTs at lower doses reduces the risk of the host producing anti-BoNT antibodies that render the treatment less effective.

TABLE 2
modified BoNT/A2 binding to glycosylated
synaptic vesicle glycoprotein 2C (SV2C)

| Hc/A2 | K_{D} (μM) | kₐ (1/Ms) | k_{d} (1/s) |
|---|---|---|---|
| HcA1wt | 0.33 | 5.61E+04 | 1.83E−02 |
| HcA2wt | 2.55 | 1.63E+04 | 4.16E−02 |
| HcA2-R1064A | 2.12 | 1.52E+04 | 3.21E−02 |
| HcA2-R1064L | 1.58 | 1.79E+04 | 2.83E−02 |
| HcA2-R1064H | 1.52 | 4.68E+03 | 7.10E−03 |
| HcA2-R1064W | 1.09 | 4.69E+03 | 5.12E−03 |
| HcA2-R1064F | 1.42 | 6.12E+03 | 8.66E−03 |
| HcA2-R1064Y | 2.07 | 4.40E+03 | 9.12E−03 |
| HcA2-S1295P | 1.31 | 1.49E+04 | 1.95E−02 |
| HcA2-P1063T/R1064H | 2.08 | 8.85E+03 | 1.84E−02 |
| HcA2-S1294R/S1295P | 0.42 | 2.50E+04 | 1.04E−02 |
| HcA2R1064H/S1294R/S1295P | 0.20 | 5.28E+04 | 1.04E−02 |
| HcA2-R1064W/S1294R/S1295P | 0.17 | 4.80E+04 | 7.99E−03 |

### Example 2

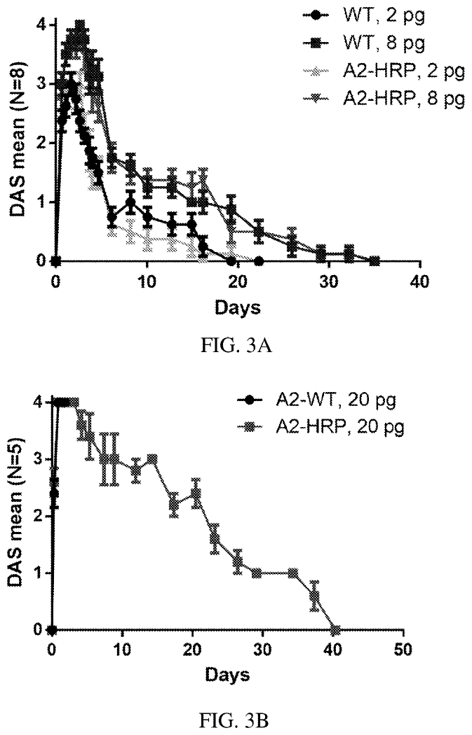

These experiments demonstrate the improved properties of the A2-HRP BoNT (R1064H/S1294R/S1295P) compared to wildtype A2 BoNT (BoNT/A2). These BoNTs were evaluated in vivo using the digit abduction score (DAS) assay in mice to measure muscle flaccidity-inducing effects. Mice were injected with either BoNT/A2 WT or BoNT/A2 mutant A2-HRLP (R1064H/S1294R/S1295P) with 2 and 8 pg injection). The A2-HRP mice exhibited similar in vivo paralytic activity when compared to BoNT/A2 wildtype. The similarity in both the 2 and 8 pg injection doses was conserved (FIG. 3A). DAS scores were also recorded following a 20 pg injection of either A2 wildtype or A2-HRP into mice (FIG. 3B). However, the WT injected mice were euthanized due to severe systemic responses. In contrast, the A2-HRP mice exhibited minor systemic response and all mice were monitored until full recovery at ˜40 days. Additionally, FIG. 3B A2-HRP (20 pg) showed approximately a 40 day DAS effect duration, whereas wildtype BoNT/A1, A2 or any other A subtypes usually have a 30 day duration.

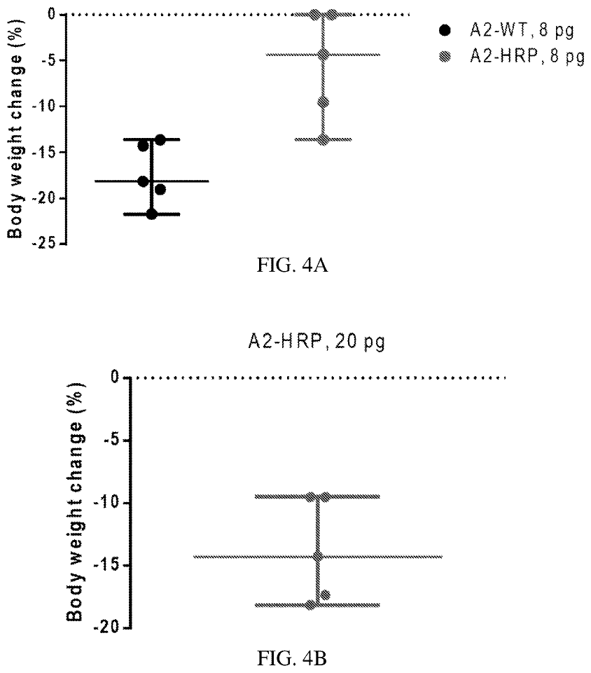

The body weight change of mice injected with 8 or 20 pg of A2-WT or A2-HRP was determined. Mice injected with 8 pg of A2-WT had a ˜20% loss of body weight, whereas, mice injected with 8 pg of A2-HRP injection resulted in ˜5% loss of body weight (FIG. 4A). Mice that received a 20 pg of A2-HRP had a ˜15% loss of body weight. Mice that received a 20 pg of A2-WT exhibited severe systemic responses and monitoring was terminated before recording body weight loss. The body weight loss of all mice was recorded, at most, two or three days after injection (FIG. 4B).

Thus, A2-HRP has longer-lasting effects broader safety injection dose range, and decreased effects on weight loss compared to the wild type BoNT/A2.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior technology or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples, which in no way should be construed as being further limiting.

What is claimed is:

1. A modified Clostridial botulinum neurotoxin (BoNT) polypeptide comprising a modified receptor binding domain of Clostridial botulinum serotype A2 (BoNT/A2) comprising one or more amino acid substitutions at positions corresponding to 1064, 1294, and 1295 of SEQ ID NO: 1.

2. The modified BoNT of claim 1, wherein the modified receptor binding domain comprises an amino acid substitution at a position corresponding to 1064 in SEQ ID NO: 1.

3. The modified BoNT of claim 2, wherein the amino acid substitution corresponds to R1064A, R1064L, R1064Y, R1064W, R1064F, or R1064H.

4. The modified BoNT of claim 2, wherein the amino acid substitution corresponds to R1064W, R1064F, or R1064H.

5. The modified BoNT of any one of claims 1-4, wherein the modified receptor binding domain comprises an amino acid substitution at a position corresponding to 1294 in SEQ ID NO: 1.

6. The modified BoNT of claim 5, wherein the amino acid substitution corresponds to S1294R.

7. The modified BoNT of any one of claims 1-6, wherein the modified receptor binding domain comprises an amino acid substitution at a position corresponding to 1295 in SEQ ID NO: 1.

8. The modified BoNT of claim 7, wherein the amino acid substitution corresponds to S1295P.

9. The modified BoNT of claim 1, wherein the modified receptor binding domain comprises amino acid substitutions at positions corresponding to 1294 and 1295 in SEQ ID NO: 1.

10. The modified BoNT of claim 9, wherein the amino acid substitutions correspond to S1294R and S1295P in SEQ ID NO: 1.

11. The modified BoNT of claim 1, wherein the modified receptor binding domain comprises amino acid substitutions at positions corresponding to 1064, 1294 and 1295 in SEQ ID NO: 1.

12. The modified BoNT of claim 11, wherein the amino acid substitutions correspond to R1064A, R1064L, R1064H, R1064W, R1064F, or R1064Y; and S1294R and S1295P in SEQ ID NO: 1.

13. The modified BoNT of claim 11, wherein the amino acid substitutions correspond to R1064H, S1294R and S1295P in SEQ ID NO: 1.

14. The modified BoNT of claim 11, wherein the amino acid substitutions correspond to R1064W, S1294R and S1295P in SEQ ID NO: 1.

15. The modified BoNT of claim 11, wherein the amino acid substitutions correspond to R1064F, S1294R and S1295P in SEQ ID NO: 1.

16. The modified BoNT polypeptide of any one of claims 1-15, wherein the modified receptor binding domain comprises an amino acid sequence that is at least 80% identical to the amino acid sequence of any one of SEQ ID NO: 12-22, SEQ ID NOs: 24-34, or SEQ ID NOs: 60-63.

17. The modified BoNT polypeptide of claim 16, wherein the modified receptor binding domain comprises the amino acid sequence of any one of SEQ ID NO: 12-22, SEQ ID NOs: 24-34, or SEQ ID NOs: 60-63.

18. The modified BoNT polypeptide of any one of claims 1-17, further comprising a protease domain and a translocation domain from BoNT/A2.

19. The modified BoNT polypeptide of any one of claims 1-17, further comprising a protease domain and a translocation domain from a second BoNT.

20. The modified BoNT polypeptide of claim 19, wherein the second BoNT is of serotype B, C, D, E, F, G, H, X, or En.

21. The modified BoNT of any one of claims 1-20, wherein the amino acid substitutions do not correspond to P1063T or P1063T and R1064H.

22. The modified BoNT of claim 1, comprising an amino acid sequence that is at least 80% identical to the amino acid sequence of SEQ ID NO: 21 or 33.

23. The modified BoNT of claim 1, comprising an amino acid sequence that is at least 95% identical to the amino acid sequence of SEQ ID NO: 21 or 33.

24. The modified BoNT of claim 1, comprising the amino acid sequence of SEQ ID NO: 21 or 33.

25. The modified BoNT polypeptide of claim 1 comprising an amino acid sequence that is at least 80% identical to SEQ ID NO: 65.

26. The modified BoNT polypeptide of claim 1 comprising an amino acid sequence that is at least 95% identical to SEQ ID NO: 65.

27. The modified BoNT polypeptide of claim 1 comprising the amino acid sequence of SEQ ID NO: 65.

28. A nucleic acid molecule comprising a polynucleotide encoding a modified BoNT polypeptide of any one of claims 1-27.

29. A nucleic acid vector comprising the nucleic acid molecule of claim 28.

30. A cell comprising the nucleic acid molecule of claim 28 or the nucleic acid vector of claim 29.

31. A cell expressing the modified BoNT polypeptide of any one of claims 1-27.

32. A method of producing a modified BoNT polypeptide, the method comprising the steps of culturing the cell of claim 30 or claim 31 under conditions wherein the modified BoNT polypeptide is produced.

33. The method of claim 32, further comprising recovering the modified BoNT polypeptide from the culture.

34. A pharmaceutical composition comprising the modified BoNT polypeptide of any one of claims 1-27.

35. The pharmaceutical composition of claim 34, further comprising a pharmaceutically acceptable excipient.

36. A kit comprising a pharmaceutical composition of claim 34 or claim 35 and directions for therapeutic administration of the pharmaceutical composition.

37. A method of treating a condition, the method comprising administering a therapeutically effective amount of the modified BoNT polypeptide of any one of claims 1-27, or the pharmaceutical composition of claim 34 or claim 35 to a subject to treat the condition.

38. The method of claim 37, wherein the condition is associated with overactive neurons or glands.

39. The method of claim 38, wherein the condition is selected from the group consisting of: depression, spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions, secretory disorders, pain from muscle spasms, headache pain, dermatological or aesthetic/cosmetic conditions, obesity/reduced appetite.

40. The method of claim 37, wherein the condition is not associated with unwanted neuronal activity.

41. The method of claim 40, wherein the condition is selected from the group consisting of: psoriasis, allergy, haemophagocytic lymphohistiocytosis, and alcoholic pancreatic disease.

42. The method of any one of any one of claims 37-39, wherein the administering is via injection to where unwanted neuronal activity is present.

43. The modified BoNT polypeptide of any one of claims 1-27, or the pharmaceutical composition of claim 34 or 35, for use in treating a condition associated with unwanted neuronal activity.

44. The modified BoNT polypeptide of any one of claims 1-27 or the pharmaceutical composition of claim 34 or 35, for use in medicine.

45. The modified BoNT polypeptide of any one of claims 1-27 or the pharmaceutical composition of claim 34 or 35, for cosmetic use.
